(19) United States
(12) Patent Application Publication
(10) Pub. No.: US 2019/0048038 A1
(43) Pub. Date: Feb. 14, 2019

(54) METHOD FOR GENERATING CELL-PENETRATING STAPLED PEPTIDES THAT LACK NONSPECIFIC MEMBRANE-LYTIC PROPERTIES FOR THERAPEUTIC TARGETING

(71) Applicant: Dana-Farber Cancer Institute, Inc., Boston, MA (US)

(72) Inventors: Loren D. Walensky, Newton Centre, MA (US); Gregory H. Bird, Pelham, NH (US)

(21) Appl. No.: 16/078,525

(22) PCT Filed: Feb. 23, 2017
(86) PCT No.: PCT/US2017/019108

§ 371 Date: Aug. 21, 2018

Related U.S. Application Data
(60) Provisional application No. 62/298,931, filed on Feb. 23, 2016.

(51) Int. Cl. C07K 1/107; C07K 1/113; C07K 19/00

(52) U.S. Cl. CPC C07K 1/1077; A61K 38/00; C07K 19/00; C07K 1/113

(57) ABSTRACT

Methods for generating cell-permeable hydrocarbon-stapled and/or stitched peptides lacking nonspecific membrane lytic properties and methods for using such peptides to target cellular proteins for experimental investigation and/or therapeutic benefit.

### CROSS-REFERENCE TO RELATED APPLICATIONS

This application claims the benefit of priority of U.S. Provisional Appl. No. 62/298,931 filed Feb. 23, 2016, the contents of which are incorporated by reference in their entirety herein.

### GOVERNMENT GRANT CLAUSE

This invention was made with government support under grant 1R35CA197583 awarded by the National Institutes of Health (NIH). The government has certain rights in the invention.

### TECHNICAL FIELD

This disclosure relates to methods for generating cell permeable hydrocarbon-stapled and/or stitched peptides lacking nonspecific membrane lytic properties and methods for using such peptides to target cellular proteins for therapeutic benefit.

### BACKGROUND

Hydrocarbon-stapled and/or stitched peptides are a class of structured bioactive ligands that have been developed to dissect, target, and/or modulate protein interactions. These peptides comprise a large and diverse array of bioactive α-helices (including evolutionarily-honed motifs) structurally stabilized by insertion of all-hydrocarbon staples¹. Stapled peptides can be effective chemical tools for investigating protein regulation, but their broader utility for modulating intracellular interactions for therapeutic benefit requires their capacity to gain entry into cells. However, the exact design features that reliably confer cellular penetrance have been elusive. In theory, stapled peptides have the potential to not only recapitulate the structure and specificity of bioactive α-helices but additionally (unlike naturally existing α-helices) have the ability to resist proteolytic degradation in vitro and in vivo^{3,29}, and, when appropriately designed, gain entrance to living cells (e.g., by a macropinocytotic mechanism)¹. The original proof of concept for synthesizing structurally-reinforced and protease-resistant “stapled” α-helices derived from the insertion of α,α-disubstituted non-natural amino acids bearing olefin tethers into an RNAse A peptide template at i, i+3; i, i+4; or i, i+7 positions, followed by ruthenium-catalyzed olefin metathesis². The first cellular application of hydrocarbon-stapled α-helices revealed their potential for cellular uptake by an energy-dependent macropinocytotic mechanism, resulting in activation of the apoptotic signaling cascade³. However, the subsequent development of a library of stapled α-helical peptides modeled after the p53 transactivation domain highlighted that not all stapled peptides are cell permeable (i.e., cell-penetrant). In fact, none of the stapled peptides in the original p53 panel were cell-penetrant⁴.

Consistent intracellular access is essential for stapled peptides to be effective when used in vivo (i.e., applied or administered to living cells or organisms, e.g., in a clinical or therapeutic context). However, to date, the development of cell-permeable stapled peptides (i.e. cell-penetrant hydrocarbon-stapled and/or stitched peptides) has generally relied on (cumulative) empirical observations and trial-and-error, both of which are inherently inefficient at identifying and/or optimizing promising peptides. Although some theories have been proposed^{1,13-15}, and specific peptides have been shown to be capable of penetrating cells under certain conditions without lysing them (and causing consequent cell death)^{4,20,53}, the lack of definitive knowledge of the criteria for generating cell-penetrant peptide analogs has presented a significant roadblock to realizing the broader utility of stapled peptides, whether as potential therapeutics in vivo or even only as experimental reagents or tools (e.g., for cellular analysis).

Further, some of the proposed theories have been shown to be inconsistent with experimental evidence. For example, a recent survey of a large and eclectic library of stapled peptides suggested that the staple itself along with overall peptide charge were the two key properties that dictated cellular penetrance¹⁴. Yet, stapled peptides with significantly net positive (+3 to +5) and negative (−4) charge have been reported to exhibit intracellular activity, in addition to the cell-penetrant stapled peptides with near-neutral net charge (−1 to +2), which predominate in the literature¹⁶. Additionally, the use of cell-impermeable stapled peptides in cellular studies has led to faulty conclusions about stapled peptide uptake and activity¹⁶⁻¹⁹. Conversely, the application of supraphysiologic doses of aggregation-prone peptide constructs can lead to nonspecific membrane lysis and consequent cytotoxicity that could be misinterpreted as a mechanistically on-target effect^{1,19}.

Thus, there is a critical need for a rigorous, quantitative roadmap for maximizing the potential to generate cell-permeable stapled peptides (i.e. cell-penetrant hydrocarbon-stapled and/or stitched peptides) with on-mechanism cellular activity. The therapeutic impact of targeting yet undruggable intracellular protein interactions using potent and specific stapled peptides—compounds that feature both (1) the large and complex surface binding capacity of antibodies, and (2) the intracellular access of small molecules—would be transformative. Stapled peptides hold remarkable promise as a novel class of compounds for dissecting and targeting protein interactions^{5,7-12}.

### SUMMARY OF THE INVENTION

We have developed an unbiased statistical method for rapid determination of optimal stapled and/or stitched peptide biophysical design parameters for maximal cell uptake propensity while minimizing nonspecific cell membrane lytic activity (a required feature for stapled and/or stitched peptides to be effective in vivo).

Key parameters that control the cellular uptake/permeability of stapled and/or stitched peptides include staple composition and placement (e.g., at the amphipathic boundary), number and placement of hydrophobic amino acid residues, and degree of α-helical content. Key parameters that contribute to non-specific cell membrane lytic activity (especially at elevated concentration) include excess hydrophobicity and positive charge (e.g., charge associated with particular amino acids and their positions). Importantly, any one of these properties with respect to cellular uptake/permeability or non-specific cell membrane lytic activity can be circumvented if another property can compensate. That is, even if, for example, the alpha-helicity of the stapled and/or stitched peptides is not optimal for cellular uptake/permeability and/or non-specific cell membrane lytic activity, that can be made up by another parameter (e.g., having the appropriate values (i.e., values that are appropriate for cellular uptake/permeability and/or non-specific cell membrane lytic activity for the peptide) for any one or more parameters selected from calculated hydrophobicity, HPLC retention time at pH 4 or 7, pI, and net charge)

The method can be applied to prepare a stapled and/or stitched peptide (e.g., a hydrocarbon stapled and/or stitched peptide) that is cell-penetrant but does not exhibit or exhibits minimal cell membrane lytic activity (e.g., relative to the unstapled/unstitched version of the peptide); or to select optimal peptides from a library of stapled and/or stitched peptides (including, e.g., staple scanning and/or point mutation libraries).

The method can be applied to select optimal peptides from a library of stapled peptides, e.g., BCL-2 homology 3 (BH3) peptides (e.g., BIM BH3 peptides).

The method can be applied to enhance the cellular uptake of pro-apoptotic p53 stapled peptides by generating a revised p53 panel bearing E to Q and D to N mutations to optimize the peptides' α-helicity and adjust the overall peptide charge from −2 to 0 and +1. The method can yield cell-penetrant analogs capable of reactivating the p53 pathway through targeted inhibition of HDM2⁴ and HDMX⁵.

The method can be applied two or more times to iteratively enhance other biophysical properties of a candidate stapled and/or stitched peptide. For example, the method can be applied to mitigate serum binding, eliminate nonspecific cell lytic activity, and further improve potency⁶.

The method can be used to select and/or optimize stapled and/or stitched peptides directed against one or more (e.g., one, two, three, four, five, six, seven, eight, nine, ten, eleven, or twelve) intracellular or extracellular targets^{3,29,30}. The method can be used to select and/or optimize stapled and/or stitched peptides effective as structured antigens for vaccination when administered to an animal (e.g., a human)³¹.

In one aspect, the disclosure features methods of making a cell-penetrant hydrocarbon-stapled and/or stitched peptide (HSP). These methods include: providing an alpha-helical peptide that binds a target protein; generating a hydrocarbon-stapled and/or stitched peptide (HSP) of the alpha-helical peptide by placing a staple and/or a stitch at an amphipathic boundary (i.e. at the hydrophobic/hydrophilic boundary) of the alpha-helical peptide, thereby generating a HSP that is cell-penetrant. In some embodiments, a hydrocarbon-stapled and stitched peptide (HSP) of the alpha-helical peptide is generated by placing a staple and a stitch at an amphipathic boundary (i.e. at the hydrophobic/hydrophilic boundary) of the alpha-helical peptide, thereby generating a HSP that is cell-penetrant. In some embodiments, a hydrocarbon-stapled and stitched peptide (HSP) of the alpha-helical peptide is generated by placing a staple or a stitch at an amphipathic boundary (i.e. at the hydrophobic/hydrophilic boundary) of the alpha-helical peptide, thereby generating a HSP that is cell-penetrant. In some embodiments, a hydrocarbon-stapled peptide (HSP) of the alpha-helical peptide is generated by placing a staple at an amphipathic boundary (i.e. at the hydrophobic/hydrophilic boundary) of the alpha-helical peptide, thereby generating a HSP that is cell-penetrant. In some embodiments, a hydrocarbon-stitched peptide (HSP) of the alpha-helical peptide is generated by placing a stitch at an amphipathic boundary (i.e. at the hydrophobic/hydrophilic boundary) of the alpha-helical peptide, thereby generating a HSP that is cell-penetrant. In some embodiments, a hydrocarbon-stapled and stitched peptide (HSP) of the alpha-helical peptide is generated by placing a staple and/or a stitch at an amphipathic boundary (i.e. at the hydrophobic/hydrophilic boundary) of the alpha-helical peptide and the HSP has, or is modified to have (e.g., by amino acid substitutions within the peptide), appropriate values (see, e.g., Table 1 below) for one or more of the parameters responsible for cellular uptake and/or preventing or inhibiting non-specific cellular lysis—i.e., parameters selected from the group consisting of hydrophobicity, HPLC retention time at pH 4 or 7, percent α-helicity, net charge, and isoelectric point. In certain embodiments, the HSP binds its target protein with the same or greater binding affinity as the alpha-helical peptide.

An alpha-helical peptide (e.g., a hydrocarbon stapled and/or stitched peptide) has a hydrophobic and a hydrophilic surface. The present disclosure relates to alpha-helical peptides (e.g., hydrocarbon stapled and/or stitched peptides) and methods of making thereof in which a staple and/or stitch is placed between the hydrophobic and hydrophilic surface, thereby extending the hydrophobic surface. Without wishing to be bound by theory, the present inventors have found that placing a staple and/or stitch that is restricted to the hydrophobic surface of the alpha-helical peptide (e.g., a hydrocarbon stapled and/or stitched peptide) results in an alpha-helical peptide (e.g., a hydrocarbon stapled and/or stitched peptide) that is, generally, not cell-penetrant.

In another aspect, the disclosure provides methods of making a cell-penetrant hydrocarbon-stapled and/or stitched peptide (HSP). These methods include: providing an alpha-helical peptide that binds a target protein; generating a hydrocarbon-stapled and/or stitched peptide (HSP) of the alpha-helical peptide by placing a staple and/or a stitch at an amphipathic boundary (i.e. at the hydrophobic/hydrophilic boundary) of the alpha-helical peptide, and modifying the alpha-helical peptide to alter (relative to the alpha-helical peptide) at least one or more (e.g., at least one, at least two, at least three) of the following biophysical parameters: calculated hydrophobicity, HPLC retention time at pH 4.0 or pH 7.0, net charge, or percent alpha-helicity, to generate a HSP that is cell-penetrant, or an HSP which shows improved cellular uptake relative to alpha-helical peptide. In some cases, the method is used to generate an HSP that is taken up by a desired cell and which does not exhibit non-specific cell lytic activity (or exhibits reduced non-specific cell lysis activity relative to the parent/unmodified alpha-helical peptide). The alpha-helical peptide can be modified by introducing one or more (e.g., 1, 2, 3, 4, 5, 6, 7, 8, 9, 10) point mutations in the amino acid sequence of the alpha-helical peptide. The modified alpha-helical peptide binds to its target protein with the same or improved binding affinity relative to the unmodified/parental alpha-helical peptide. In some embodiments, a hydrocarbon-stapled and stitched peptide (HSP) of the alpha-helical peptide is generated by placing a staple and a stitch at an amphipathic boundary of the alpha-helical peptide. In some embodiments, a hydrocarbon-stapled and stitched peptide (HSP) of the alpha-helical peptide is generated by placing a staple or a stitch at an amphipathic boundary of the alpha-helical peptide. In some embodiments, a hydrocarbon-stapled peptide (HSP) of the alpha-helical peptide is generated by placing a staple at an amphipathic boundary (i.e. at the hydrophobic/hydrophilic boundary) of the alpha-helical peptide. In some embodiments, a hydrocarbon-stitched peptide (HSP) of the alpha-helical peptide is generated by placing a stitch at an amphipathic boundary (i.e. at the hydrophobic/hydrophilic boundary) of the alpha-helical peptide, thereby generating a HSP that is cell-penetrant.

In some embodiments of the above aspects, methods of making a cell-penetrant hydrocarbon stapled and/or stitched peptide (HSP) include inserting a staple and/or a stitch at a position of (i and i+3), (i and i+4), or (i and i+7), wherein the positions of the staple and/or stitch are replaced (e.g., substituted or modified) with non-natural amino acids (e.g., amino acids with olefinic side chains). In some embodiments, a cell-penetrant hydrocarbon stapled and/or stitched peptide has a single (i and i+3), (i and i+4), or (i and i+7), staple/stitch, or multiple staples/stitches (e.g., two, three, four, or five).

In some embodiments, the alpha-helical peptide and/or HSP is 6 to 100 amino acids in length. In some cases, the alpha-helical peptide and/or HSP is 6 to 90, 6 to 80, 6 to 70, 6 to 60, 6 to 50, or 6 to 40 amino acids in length (e.g., 10 to 20 amino acids in length, 10 to 30 amino acids in length, 10 to 40 amino acids in length, 20 to 30 amino acids in length, 20 to 40 amino acids in length, 20 to 50 amino acids in length, or 20 to 60 amino acids in length). In some embodiments, the staple and/or stitch extends the hydrophobic surface beyond the target protein binding surface.

In some embodiments, the HSP is derivatized at the N-terminus with a fluorophore (e.g., FITC-βAla or acetyl). In some embodiments, the HSP is derivatized at the N-terminus with FITC-βAla or acetyl. To test cell-penetrance, one could use the fluorophore-attached HSP to determine the total FITC intensity (TIFI) using any of the methods described herein. In some embodiments, the HSP has a TIFI that is greater than 0.5×10⁶ (e.g., greater than 0.7×10⁶, greater than 0.8×10⁶, greater than 1.0×10⁶, greater than 1.5×10⁶, greater than 2.0×10⁶, greater than 2.5×10⁶, greater than 3.0×10⁶; 1.0×10⁶, 3×10⁶, or 6×10⁶). In some embodiments, the HSP has a TIFI that is greater than 1.5×10⁶. In some embodiments, the HSP has a TIFI that is greater than 3.0×10⁶.

In certain embodiments, the HSP has a calculated hydrophobicity that is greater than 0.5. In certain embodiments, the HSP has a calculated hydrophobicity that is greater than 0.6. In certain embodiments, the HSP has a calculated hydrophobicity that is about 0.5, about 0.6, about 0.7, about 0.8, or about 0.9. In other embodiments, the HSP has a calculated hydrophobicity that is 0.5, 0.6, 0.7, 0.8, or 0.9.

In certain embodiments, the HSP has a high performance liquid chromatography (HPLC) retention time of 9.56 minutes or greater at pH 7 or pH 4. In certain embodiments, the HSP has a HPLC retention time of 9.7 minutes to 11.2 minutes at pH 7 or pH 4. In certain embodiments, the HSP has a HPLC retention time of 9.56 minutes or greater at pH 7. In certain embodiments, the HSP has a HPLC retention time of 9.56 minutes to 11.2 minutes at pH 7. Under routine conditions, peptides are purified by HPLC at pH 4. Therefore, under routine conditions, hydrocarbon-stapled and/or stitched peptides (e.g., a hydrocarbon-stapled peptide, a hydrocarbon-stitched peptide, a hydrocarbon peptide that comprises a staple and a stitch) may be purified by HPLC at pH 4. For example, in certain embodiments, the HSP has a HPLC retention time greater than 11.0 minutes at pH 4 (e.g., a HPLC retention time of about 11.0 minutes to about 12.5 minutes at pH 4, a HPLC retention time of about 11.2 minutes to about 12.5 minutes at pH 4, or a HPLC retention time of about 11.5 minutes to about 12.5 minutes at pH 4).

In some embodiments, the HSP has a percent α-helicity of 61% to 86% (e.g., 61% to 65%, 61% to 70%, 61% to 75%, 61% to 80%, 61% to 85%, 65% to 70%, 65% to 75%, 65% to 80%, 65% to 85%, 65% to 86%).

In some embodiments, the HSP has a net charge of +4 to −3. In other embodiments, the HSP has a net charge of +3 to −3. In certain embodiments, the HSP has a net charge of +2 to −2. In some embodiments, the HSP has a net charge of +2 to −1 (e.g., a net charge of +2, a net charge of +1, a net charge of 0, a net charge of −1).

In certain embodiments, the HSP is derived from an alpha-helical peptide from an anti-apoptotic or a pro-apoptotic BCL-2 family protein (e.g., BCL-XL, BCL-W, MCL-1, BLC-B, BID, BIM, BAD, NOXA, PUMA, BAX, BAK, or BOK). In certain embodiments, the HSP is derived from an alpha-helical peptide from an anti-apoptotic BCL-2 family protein (e.g., BCL-XL, BCL-W, MCL-1, or BLC-B). In certain embodiments, the HSP is derived from an alpha-helical peptide from a pro-apoptotic BCL-2 family protein (e.g., BID, BIM, BAD, NOXA, PUMA, BAX, BAK, or BOK). In certain embodiments, the HSP is derived from a BCL-2 homology 3 (BH3) peptide. In certain embodiments, the HSP is derived from an alpha-helical peptide from p53. In certain embodiments, the HSP is derived from an alpha-helical peptide from SOS.

In some embodiments of the above aspects, the HSP binds its target protein with the same or improved binding affinity relative to the unmodified/parental alpha-helical peptide.

In yet another aspect, the disclosure provides cell-penetrant hydrocarbon-stapled and/or stitched peptides (HSP) that include a hydrocarbon-stapled and/or stitch peptide (HSP); wherein a staple and/or stitch is located at an amphipathic boundary of the HSP, and wherein the HSP is cell-penetrant. In certain embodiments, a staple is located at an amphipathic boundary of the HSP, and wherein the HSP is cell-penetrant. In certain embodiments, a stitch is located at an amphipathic boundary of the HSP, and wherein the HSP is cell-penetrant. In certain embodiments, a staple and a stich are placed at an amphipathic boundary of the HSP, and wherein the HSP is cell-penetrant.

In some embodiments, a cell-penetrant hydrocarbon stapled and/or stitched peptide (HSP) includes a staple and/or a stitch at a position of (i and i+3), (i and i+4), or (i and i+7), wherein the positions of the staple and/or stitch are replaced (e.g., substituted or modified) with non-natural amino acids (e.g., amino acids with olefinic side chains). In some embodiments, a cell-penetrant hydrocarbon stapled and/or stitched peptide has a single (i and i+3), (i and i+4), or (i and i+7), staple/stitch, or multiple staples/stitches (e.g., two, three, four, or five).

In some embodiments, the HSP is 6 to 100 amino acids in length. In some cases, the alpha-helical peptide and/or HSP is 6 to 90, 6 to 80, 6 to 70, 6 to 60, 6 to 50, or 6 to 40 amino acids in length (e.g., 10 to 20 amino acids in length, 10 to 30 amino acids in length, or 10 to 40 amino acids in length, 20 to 30 amino acids in length, 20 to 40 amino acids in length, 20 to 50 amino acids in length, or 20 to 60 amino acids in length). In some embodiments, the staple and/or stitch extends the hydrophobic surface beyond the target protein binding surface.

In some embodiments, the HSP is derivatized at the N-terminus with a fluorophore (e.g., FITC-βAla or acetyl). In some embodiments, the HSP is derivatized at the N-terminus with FITC-βAla or acetyl. In certain embodiments, the HSP has a total internalized FITC intensity (TIFI) that is greater than 0.5×10⁶ (e.g., greater than 0.7×10⁶, greater than 0.8×10⁶, greater than 1.0×10⁶, greater than 1.5×10⁶, greater than 2.0×10⁶, greater than 2.5×10⁶, or greater than 3.0×10⁶; 1.0×10⁶, 3×10⁶, or 6×10⁶). In some embodiments, the HSP has a TIFI that is greater than 3.0×10⁶.

In certain embodiments, the HSP has a calculated hydrophobicity that is greater than 0.5. In certain embodiments, the HSP has a calculated hydrophobicity that is greater than 0.6. In certain embodiments, the HSP has a calculated hydrophobicity that is about 0.5, about 0.6, about 0.7, about 0.8, or about 0.9. In other embodiments, the HSP has a calculated hydrophobicity that is 0.5, 0.6, 0.7, 0.8, or 0.9.

In certain embodiments, the HSP has a high performance liquid chromatography (HPLC) retention time of 9.56 minutes or greater at pH 7 or pH 4. In certain embodiments, the HSP has a HPLC retention time of 9.7 minutes to 11.2 minutes at pH 7 or pH 4. In certain embodiments, the HSP has a HPLC retention time of 9.56 minutes or greater at pH 7. In certain embodiments, the HSP has a HPLC retention time of 9.56 minutes to 11.2 minutes at pH 7. Under routine conditions, peptides are purified by HPLC at pH 4. Therefore, under routine conditions, hydrocarbon-stapled and/or stitched peptides (e.g., a hydrocarbon-stapled peptide, a hydrocarbon-stitched peptide, or a hydrocarbon peptide that comprises a staple and a stitch) may be purified by HPLC at pH 4. For example, in certain embodiments, the HSP has a HPLC retention time greater than 11.0 minutes at pH 4 (e.g., a HPLC retention time of about 11.0 minutes to about 12.5 minutes at pH 4, a HPLC retention time of about 11.2 minutes to about 12.5 minutes at pH 4, or a HPLC retention time of about 11.5 minutes to about 12.5 minutes at pH 4).

In some embodiments, the HSP has a percent α-helicity of 61% to 86% (e.g., 61% to 65%, 61% to 70%, 61% to 75%, 61% to 80%, 61% to 85%, 65% to 70%, 65% to 75%, 65% to 80%, 65% to 85%, 65% to 86%).

In some embodiments, the HSP has a net charge of +4 to −3. In other embodiments, the HSP has a net charge of +3 to −3. In certain embodiments, the HSP has a net charge of +2 to −2. In some embodiments, the HSP has a net charge of +2 to −1 (e.g., a net charge of +2, a net charge of +1, a net charge of 0, or a net charge of −1).

In certain embodiments, the HSP is derived from an alpha-helical peptide from an anti-apoptotic or a pro-apoptotic BCL-2 family protein (e.g., BCL-XL, BCL-W, MCL-1, BLC-B, BID, BIM, BAD, NOXA, PUMA, BAX, BAK, or BOK). In certain embodiments, the HSP is derived from an alpha-helical peptide from an anti-apoptotic BCL-2 family protein (e.g., BCL-XL, BCL-W, MCL-1, or BLC-B). In certain embodiments, the HSP is derived from an alpha-helical peptide from a pro-apoptotic BCL-2 family protein (e.g., BID, BIM, BAD, NOXA, PUMA, BAX, BAK, or BOK). In certain embodiments, the HSP is derived from a BCL-2 homology 3 (BH3) peptide. In certain embodiments, the HSP is derived from an alpha-helical peptide from p53. In certain embodiments, the HSP is derived from an alpha-helical peptide from SOS.

In some embodiments, the HSP has one or more of: a calculated hydrophobicity that is greater than 0.5 (e.g., greater than 0.6, greater than 0.7, greater than 0.8; about 0.5, about 0.6, about 0.7, about 0.8, about 0.9, 0.6, 0.7, 0.8, or 0.9), a HPLC retention time of 9.2 or greater at pH 7 or pH 4 (e.g., a HPLC retention time of 9.56 or greater at pH 7, a HPLC retention time of 10.0 or greater at pH 7, a HPLC retention time of 11.0 or greater at pH 7, a HPLC retention time of 9.56 or greater at pH 4, a HPLC retention time of 10.0 or greater at pH 4, or a HPLC retention time of 11.0 or greater at pH 4), a percent α-helicity of 40% to 90% (e.g., 40% to 85%, 50% to 85%, 60% to 85%, 61% to 86%, 70% to 85%, 80% to 90%), a pI of less than 9.75 (e.g., a pI of about 7.0 to about 9.34, a pI of about 7.5 to about 9.34; a pI of about 7.8 to about 9.34; a pI of about 8.0 to about 9.34; a pI of about 8.2 to about 9.34; a pI of about 8.5 to about 9.34; a pI of about 8.8 to about 9.34 a pI of about 8.8 to about 9.74; a pI of about 7.8 to about 9.74; or a pI of about 7.0 to about 9.34), and a net charge of +4 to −3 (e.g., a net a charge of +3 to −3, a net charge of +2 to −2, a net charge of +2 to −1, a net charge of +2, a net charge of +1, a net charge of 0, or a net charge of −1).

The HSP is 6 to 100 amino acids in length (e.g., 6 to 90, 6 to 80, 6 to 70, 6 to 60, 6 to 50, or 6 to 40 amino acids in length (e.g., 10 to 20 amino acids in length, 10 to 30 amino acids in length, or 10 to 40 amino acids in length, 20 to 30 amino acids in length, 20 to 40 amino acids in length, 20 to 50 amino acids in length, or 20 to 60 amino acids in length).

In some embodiments, the HSP has a calculated hydrophobicity that is between 0.5 and 0.9 (e.g., 0.5, 0.6, 0.7, 0.8, 0.9) and has a HPLC retention time of 9.56 or greater at pH7 or pH4 (e.g., a HPLC retention time of 9.56 or greater at pH7, or a HPLC retention time of 11.0 or greater at pH4). In some embodiments, the HSP has a HPLC retention time of 9.56 or greater at pH7 or pH4 (e.g., a HPLC retention time of 9.56 or greater at pH7, or a HPLC retention time of 11.0 or greater at pH4) and a percent α-helicity of 40^{%} to 90% (e.g., 61% to 86%). In some embodiments, the cell-penetrant HSP may have a combination of one or more (e.g., one, two, three, four, or five) of the biophysical properties described above. Table 1 below provides a summary of the key biophysical parameters and values of cell-penetrant hydrocarbon stapled and/or stitched peptides as well as cell-penetrant hydrocarbon stapled and/or stitched peptides that are not non-specifically cell lytic.

TABLE 1
Biophysical Parameters

| Biophysical | Cell-penetrant hydrocarbon stapled and/or stitched |
|---|---|
| Parameter | peptide |
| Total Internalized | greater than 0.5 × 10⁶ (e.g., 0.5 × 10⁶ to |
| FITC Intensity | 6.0 × 10⁶) |
| (TIFI) | |
| Hydrophobicity | greater than 0.5 (e.g., 0.5 to 0.9) |
| HPLC retention | 9.2 minutes or greater (e.g., 9.2 to 11.2 minutes) |
| time at pH 7 or pH 4 | |
| α-helicity | about 40% to about 90%** |
| pI | less than 9.76** |
| Net charge | +2 to −1 (e.g., +4 to −3) |

**indicates that these values are different for a cell-penetrant hydrocarbon stapled and/or stitched peptide that does not exhibit t non-specific cell lytic activity: α-helicity ranges for the latter are about 21% to about 96%, and pI is less than 9.76 (8.8 to 9.34).
⁺ If both pI is greater than 9.76 and HPLC retention time is greater than 9.78 min, chances of non-specific membrane lysis greatly increase.

In one aspect, the disclosure features methods of selecting a hydrocarbon-stapled and/or stitched peptide (HSP), the method involving: providing a library of HSPs; assessing the hydrophobicity of the HSPs in the library; and selecting an HSP having an overall cellular uptake-facilitating level of hydrophobicity. The hydrophobic surface area of the selected HSP can extend beyond the interaction site of the selected HSP with its target. The overall cellular uptake-facilitating level of hydrophobicity can correspond to an HPLC retention time of, e.g., about 9.7 to about 11.2 minutes. The selected HSP can contain a staple and/or a stitch at the amphipathic boundary of the HSP. The method can further include assessing the charge or isoelectric point of the HSPs in the library and selecting an HSP having an overall cellular uptake-facilitating charge or isoelectric point. The isoelectric point can be, e.g., about 8.8 to about 9.34. Moreover, the method can further include assessing the cell permeability of the HSPs in the library and selecting an HSP with high cell permeability. In addition, the method can further include assessing the cell lytic activity of the HSPs in the library and selecting an HSP with low or no cell lytic activity. Furthermore, the method can further include assessing the α-helicity of the HSPs in the library and selecting an HSP having an overall cellular uptake-facilitating level of α-helicity. The α-helicity can be, e.g., 61% to 86%. In the method, the library can be, e.g., a staple walk library or a point mutant library.

The disclosure also provides a method of selecting a hydrocarbon-stapled and/or stitched peptide (HSP), the method including: providing a library of HSPs; assessing the hydrophobicity of the HSPs in the library; selecting one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, thirty, forty, fifty, sixty, seventy, eighty, ninety, one hundred, two hundred, three hundred, four hundred, five hundred, or a thousand) HSPs having an overall cellular uptake-facilitating level of hydrophobicity; assessing at least one of the α-helicity, cell permeability, charge, isoelectric point, and/or cell membrane lytic activity of the one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, thirty, forty, fifty, sixty, seventy, eighty, ninety, one hundred, two hundred, three hundred, four hundred, five hundred, or a thousand) selected HSPs; and further selecting one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, thirty, forty, fifty, sixty, seventy, eighty, ninety, one hundred, two hundred, three hundred, four hundred, five hundred, or a thousand) HSPs with an overall cellular uptake-facilitating α-helicity, an overall cellular uptake-facilitating charge, an overall cellular uptake-facilitating isoelectric point, high cell permeability, and/or low cell lytic activity.

Also featured by this disclosure relates to methods of identifying tolerated sites for diversification within a hydrocarbon-stapled and/or stitched peptide (HSP), the method involving: providing a an initial HSP; substituting one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, or thirty) amino acids in the initial HSP with alanine, glutamate, aspartate, arginine, and/or lysine to generate a library of HSP variants; assessing at least one of hydrophobicity, α-helicity, cell permeability, charge, isoelectric point, and/or cell lytic activity of the library of HSP variants; and selecting one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, thirty, forty, fifty, sixty, seventy, eighty, ninety, one hundred, two hundred, three hundred, four hundred, five hundred, or a thousand) HSP variants with hydrophobicity, α-helicity, cell permeability, charge, isoelectric point, and/or cell lytic activity similar to that of the initial HSP.

Another feature provided by this disclosure is a method of making a hydrocarbon-stapled and/or stitched peptide (HSP), the method including synthesizing an HSP having an overall cellular uptake-facilitating level of hydrophobicity. The hydrophobic surface area of the HSP can extend beyond the interaction site of the HSP with its target. The level of hydrophobicity can correspond to an HPLC retention time of, e.g., about 9.7 to about 11.2 minutes. The HSP can have an overall cellular uptake-facilitating level of α-helicity. The overall α-helicity of the HSP can be, e.g., 61% to 86%. The HSP can have a cellular uptake-facilitating isoelectric point. The isoelectric point can be, e.g., about 8.8 to about 9.34. The HSP can contain a staple and/or a stitch at the amphipathic boundary of the HSP. The HSP can have high cell permeability. In addition, the HSP can have low or no cell lytic activity.

Another method featured by the disclosure is method of making a hydrocarbon-stapled and/or stitched peptide (HSP), the method involving: synthesizing an HSP having an overall cellular uptake-facilitating level of hydrophobicity (e.g., the levels exemplified above), an overall cellular uptake-facilitating level of α-helicity (e.g., the levels exemplified above), and a cellular uptake-facilitating isoelectric point (e.g., the pIs exemplified above). Moreover, the HSP can include a staple and/or a stitch at the amphipathic boundary of the HSP, the HSP can have high cell permeability, and the HSP can have low or no cell lytic activity. It is understood that the disclosure also features methods in which the HSP produced has less than all (e.g., one, two, three, four, or five) of the above properties (i.e., an overall cellular uptake-facilitating level of hydrophobicity, an overall cellular uptake-facilitating level of α-helicity, a cellular uptake-facilitating isoelectric point, a staple and/or a stitch at the amphipathic boundary of the HSP, high cell permeability, and low or no cell lytic activity).

The disclosure relates to methods of determining the cell-penetrance of a hydrocarbon-stapled and/or stitched peptide (HSP). These methods include: (a) providing a hydrocarbon-stapled and/or stitched peptide (HSP); (b) determining at least one biophysical property of the HSP; wherein the at least one biophysical property is hydrophobicity, high performance liquid chromatography (HPLC) retention time, percent α-helicity, or net charge; and (c) determining the cell-penetrance of the HSP based on the at least one biophysical property.

In some embodiments, a hydrocarbon stapled and/or stitched peptide (HSP) includes a staple and/or a stitch at a position of (i and i+3), (i and i+4), or (i and i+7), wherein the positions of the staple and/or stitch are replaced (e.g., substituted or modified) with non-natural amino acids (e.g., amino acids with olefinic side chains). In some embodiments, a cell-penetrant hydrocarbon stapled and/or stitched peptide has a single (i and i+3), (i and i+4), or (i and i+7), staple/stitch, or multiple staples/stitches (e.g., two, three, four, or five).

In some embodiments, the alpha-helical peptide and/or HSP is 6 to 100 amino acids in length. In some cases, the alpha-helical peptide and/or HSP is 6 to 90, 6 to 80, 6 to 70, 6 to 60, 6 to 50, or 6 to 40 amino acids in length (e.g., 10 to 20 amino acids in length, 10 to 30 amino acids in length, 10 to 40 amino acids in length, 20 to 30 amino acids in length, 20 to 40 amino acids in length, 20 to 50 amino acids in length, or 20 to 60 amino acids in length). In some embodiments, the staple and/or stitch extends the hydrophobic surface beyond the target protein binding surface.

In some embodiments, the at least one biophysical property of the HSP is hydrophobicity, and the method comprises: determining hydrophobicity of the HSP; and determining that either: (i) the HSP has a calculated hydrophobicity that is greater than 0.5, and the HSP is cell-penetrant, or (ii) the HSP had a calculated hydrophobicity that is less than 0.5, and the HSP is not cell-penetrant. In some embodiments, the HSP has a calculated hydrophobicity that is greater than 0.6, and the HSP is cell-penetrant. In some embodiments, the HSP has a calculated hydrophobicity that is greater than 0.7, and the HSP is cell-penetrant. In some embodiments, the HSP has a calculated hydrophobicity that is greater than 0.8, and the HSP is cell-penetrant. In certain embodiments, the HSP has a calculated hydrophobicity that is about 0.5, about 0.6, about 0.7, about 0.8, or about 0.9, and is cell-penetrant. In other embodiments, the HSP has a calculated hydrophobicity that is 0.5, 0.6, 0.7, 0.8, or 0.9, and is cell-penetrant. In some embodiments, the HSP had a calculated hydrophobicity that is less than 0.4, and the HSP is not cell-penetrant.

In certain embodiments, the at least one biophysical property of the HSP is HPLC retention time, and determining that either (i) the HPLC retention time of the HSP is less than 9.56 minutes at pH 7 or pH 4 (e.g., less than 9.56 minutes at pH 7, or less than 11.0 minutes at pH 4), and the HSP is not cell-penetrant; or (ii) the HPLC retention time of the HSP is equal to or greater than 9.56 minutes at pH 7 or pH 4 (e.g., a HPLC retention time equal to or greater than 9.56 minutes at pH 7, a HPLC retention time of 9.56 minutes to 11.2 minutes at pH7, or a HPLC retention time greater than 11.0 minutes at pH 4 (e.g., a HPLC retention time of about 11.0 minutes to about 12.5 minutes at pH4, a HPLC retention time of about 11.2 minutes to about 12.5 minutes at pH4, or a HPLC retention time of about 11.5 minutes to about 12.5 minutes at pH4), and the HSP is cell-penetrant.

In some embodiments, the at least one biophysical property of the HSP is percent α-helicity, and determining that either (i) the percent α-helicity of the HSP is less than 20% or greater than 90%, and the HSP is not cell-penetrant; or (ii) the percent α-helicity of the HSP is 61% to 86%, and the HSP is cell-penetrant. In some embodiments, the percent α-helicity of the HSP is less than 20% (e.g., less than 15%, less than 10%), and the HSP is not cell-penetrant. In some embodiments, the percent α-helicity of the HSP is greater than 90% (e.g., greater than 95%, greater than 96%, greater than 98%, greater than 99%, 90%, 91%, 92%, 93%, 94%, 95%, 96%), and the HSP is not cell-penetrant. In some embodiments, the percent α-helicity of the HSP is 61% to 86% (e.g., 61% to 65%, 61% to 70%, 61% to 75%, 61% to 80%, 61% to 85%, 65% to 70%, 65% to 75%, 65% to 80%, 65% to 85%, 65% to 86%), and the HSP is cell-penetrant.

In some embodiments, the at least one biophysical property of the HSP is net charge, and the method further includes determining the net charge of the HSP is +2 to −1 (e.g., the net charge of the HSP is +2, the net charge of the HSP is +1, the net charge of the HSP is 0, or the net charge of the HSP is −1), and the HSP is cell-penetrant. In some embodiments, the at least one biophysical property of the HSP is net charge, and the method further includes determining; and wherein the net charge of the HSP is 0 or +1. In some embodiments, the at least one biophysical property of the HSP is net charge, and the method further includes determining; and wherein the net charge of the HSP is 0. In some embodiments, the at least one biophysical property of the HSP is net charge, and the method further includes determining; and wherein the net charge of the HSP is +1. In some embodiments, the at least one biophysical property of the HSP is net charge, and the method further includes determining; and wherein the net charge of the HSP is +4 to −3 (e.g., +3 to −3).

In some embodiments, the HSP is derivatized at the N-terminus with a fluorophore (e.g., FITC-3Ala or acetyl). In some embodiments, the HSP is derivatized at the N-terminus with FITC-3Ala or acetyl. To test cell-penetrance, one could use the fluorophore-attached HSP to determine the total FITC intensity (TIFI) using any of the methods described herein. In some embodiments, the HSP has a TIFI that is greater than 0.5×10⁶ (e.g., greater than 0.7×10⁶, greater than 0.8×10⁶, greater than 1.0×10⁶, greater than 1.5×10⁶, greater than 2.0×10⁶, greater than 2.5×10⁶, or greater than 3.0×10⁶; 1.0×10⁶, 3×10⁶, or 6×10⁶). In some embodiments, the HSP has a TIFI that is greater than 1.5×10⁶. In some embodiments, the HSP has a TIFI that is greater than 3.0×10⁶. In some embodiments, a TIFI that is less than 0.5×10⁶ (e.g., less than 4×10⁵, less than 2.0×10⁵, less than 1×10⁵, less than 0.5×10⁵, less than 4×10⁴, less than 2.0×10⁴, less than 1×10⁴, less than 0.5×10⁴, less than 4×10³, less than 2.0×10³, less than 1×10³, or less than 0.5×10³) indicates that the HSP is not cell-penetrant.

The disclosure also features methods of optimizing cell-penetrance of a hydrocarbon-stapled and/or stitched peptide. These methods involve: (a) providing a first hydrocarbon-stapled and/or stitched peptide (HSP) that binds a target protein; (b) generating a second HSP that is identical to the first HSP except at one or more (e.g., one, two, three, four, five, six, seven, eight, nine, ten) amino acid positions, wherein the second HSP binds to the same target as the first HSP, and has at least one altered biophysical property compared to the first HSP; wherein the at least one biophysical property is selected from the group consisting of: hydrophobicity, high performance liquid chromatography (HPLC) retention time, percent α-helicity, or net charge; and wherein (i) the HPLC retention time of the second HSP is altered (e.g., increased or decreased) compared to the first HSP, and the second HSP has improved cellular uptake as compared to the first HSP; (ii) the hydrophobicity of the second HSP is altered (e.g., increased or decreased) compared to the first HSP, and the second HSP has improved cellular uptake as compared to the first HSP; (iii) the net charge of the second HSP is altered (e.g., increased or decreased) compared to the first HSP, and the second HSP has improved cellular uptake as compared to the first HSP; or (iv) the percent α-helicity of the second HSP is altered (e.g., increased or decreased) compared to the first HSP, and the second HSP has improved cellular uptake as compared to the first HSP.

In some embodiments, a cell-penetrant hydrocarbon stapled and/or stitched peptide (HSP) includes a staple and/or a stitch at a position of (i and i+3), (i and i+4), or (i and i+7), wherein the positions of the staple and/or stitch are replaced (e.g., substituted or modified) with non-natural amino acids (e.g., amino acids with olefinic side chains). In some embodiments, a cell-penetrant hydrocarbon stapled and/or stitched peptide has a single (i and i+3), (i and i+4), or (i and i+7), staple/stitch, or multiple staples/stitches (e.g., two, three, four, or five).

In some embodiments, the HSP is 6 to 100 amino acids in length. In some cases, the alpha-helical peptide and/or HSP is 6 to 90, 6 to 80, 6 to 70, 6 to 60, 6 to 50, or 6 to 40 amino acids in length (e.g., 10 to 20 amino acids in length, 10 to 30 amino acids in length, or 10 to 40 amino acids in length, 20 to 30 amino acids in length, 20 to 40 amino acids in length, 20 to 50 amino acids in length, or 20 to 60 amino acids in length). In some embodiments, the staple and/or stitch extends the hydrophobic surface beyond the target protein binding surface.

In certain embodiments, the second HSP has increased retention time as compared to the first HSP, and the second HSP has improved cellular uptake as compared to the first HSP. In certain embodiments, the second HSP has an increased net charge as compared to the first HSP, and the second HSP has improved cellular uptake as compared to the first HSP. In certain embodiments, the second HSP has a reduced net charge as compared to the first HSP, and the second HSP has improved cellular uptake as compared to the first HSP. In certain embodiments, the second HSP binds to the same target as the first HSP with the same or greater binding affinity to the target as compared to the first HSP. In certain embodiments, the second HSP binds to the target protein with a binding affinity of less than 100 nM (e.g., less than 90 nM, less than 80 nM, less than 70 nM, less than 60 nM, less than 50 nM, less than 40 nM, about 1 nM to about 10 nM, about 1 nM to about 20 nM, about 1 nM to about 30 nM, about 1 nM to about 40 nM, about 1 nM to about 50 nM, about 1 nM to about 60 nM, about 1 nM to about 70 nM, about 1 nM to about 80 nM, about 1 nM to about 90 nM, about 1 nM to about 100 nM, about 10 nM to about 20 nM, about 10 nM to about 30 nM, about 10 nM to about 40 nM, about 10 nM to about 50 nM, about 10 nM to about 100 nM, about 20 nM to about 100 nM, about 30 nM to about 100 nM, about 40 nM to about 100 nM, or about 50 nM to about 100 nM).

In some embodiments, the second HSP has the same or reduced effect on non-specific cell lysis.

In one aspect, the disclosure relates to methods of selecting a cell-penetrant hydrocarbon-stapled and/or stitched peptide (HSP) that does not exhibit non-specific cell lytic activity. These methods involve: (a) providing a hydrocarbon-stapled and/or stitched alpha-helical peptide (HSP) that binds a target protein; (b) determining percent α-helicity or isoelectric point (pI) of the HSP; and (c) selecting the HSP as exhibiting non-specific cell lytic activity, when the HSP has: (i) an isoelectric point (pI) that is less than 9.76, and (ii) a percent α-helicity that ranges from 21% to 96%.

In some embodiments, a hydrocarbon stapled and/or stitched peptide (HSP) includes a staple and/or a stitch at a position of (i and i+3), (i and i+4), or (i and i+7), wherein the positions of the staple and/or stitch are replaced (e.g., substituted or modified) with non-natural amino acids (e.g., amino acids with olefinic side chains). In some embodiments, a cell-penetrant hydrocarbon stapled and/or stitched peptide has a single (i and i+3), (i and i+4), or (i and i+7), staple/stitch, or multiple staples/stitches (e.g., two, three, four, or five).

In some embodiments, the HSP is 6 to 100 amino acids in length. In some cases, the alpha-helical peptide and/or HSP is 6 to 90, 6 to 80, 6 to 70, 6 to 60, 6 to 50, or 6 to 40 amino acids in length (e.g., 10 to 20 amino acids in length, 10 to 30 amino acids in length, or 10 to 40 amino acids in length, 20 to 30 amino acids in length, 20 to 40 amino acids in length, 20 to 50 amino acids in length, or 20 to 60 amino acids in length). In some embodiments, the staple and/or stitch extends the hydrophobic surface beyond the target protein binding surface.

In some embodiments, the method further includes determining that the hydrophobicity of the HSP is greater than 0.5 (e.g., greater than 0.6). In certain embodiments, the HSP has a calculated hydrophobicity that is about 0.5, about 0.6, about 0.7, about 0.8, or about 0.9. In other embodiments, the HSP has a calculated hydrophobicity that is 0.5, 0.6, 0.7, 0.8, or 0.9.

In some embodiments, the method further includes determining that the HPLC retention time of the HSP is equal to or greater than 9.56 minutes at pH 7 or pH 4 (e.g., a HPLC retention time equal to or greater than 9.56 minutes at pH 7, a HPLC retention time of 9.56 minutes to 11.2 minutes at pH7, or a HPLC retention time greater than 11.0 minutes at pH 4 (e.g., a HPLC retention time of about 11.0 minutes to about 12.5 minutes at pH4, a HPLC retention time of about 11.2 minutes to about 12.5 minutes at pH4, or a HPLC retention time of about 11.5 minutes to about 12.5 minutes at pH4).

In some embodiments, the method further includes determining that the percent α-helicity of the HSP is 61% to 86% (e.g., 61% to 65%, 61% to 70%, 61% to 75%, 61% to 80%, 61% to 85%, 65% to 70%, 65% to 75%, 65% to 80%, 65% to 85%, 65% to 86%).

In some embodiments, the method further includes determining that the pI of the HSP is less than 9.76 (e.g., less than 9.75, less than 9.7, less than 9.6, less than 9.4, less than 9.2, less than 9.0, less than 8.8, less than 8.6, less than 8.5, less than 8.0, less than 7.5, less than 7.0; less than 9.75 but greater than 6.0, less than 9.75 but greater than 7.0, less than 9.75 but greater than 8.0; about 7.0 to about 9.34, a pI of about 7.5 to about 9.34; a pI of about 7.8 to about 9.34; a pI of about 8.0 to about 9.34; a pI of about 8.2 to about 9.34; a pI of about 8.5 to about 9.34; a pI of about 8.8 to about 9.34; a pI of about 8.8 to about 9.74; a pI of about 7.8 to about 9.74; a pI of about 7.0 to about 9.34; 7.0, 7.1, 7.2, 7.5, 8.0, 8.1, 8.2, 8.3, 8.4, 8.5, 8.6, 8.7, 8.8, 8.9, 9.0, 9.1 9.2, 9.3, 9.4, 9.5, 9.6, 9.7)

In some embodiments, the method further includes determining that the net charge of the HSP is +4 to −3 (e.g., the net charge of the HSP is +3 to −3, the net charge of the HSP is +2 to −2, the net charge of the HSP is +2 to −1; the net charge of the HSP is +2, the net charge of the HSP is +1, the net charge of the HSP is 0, or the net charge of the HSP is −1). In some embodiments, the method further includes determining that the net charge of the HSP is 0 or +1.

In one aspect, the disclosure features methods of determining the cell-penetrance and non-specific cell lysis activity of a hydrocarbon-stapled and/or stitched peptide (HSP). These methods involve: (a) providing a hydrocarbon-stapled and/or stitched alpha-helical peptide (HSP) that binds a target protein; (b) determining at least one biophysical property of the HSP; wherein the at least one biophysical property is hydrophobicity, high performance liquid chromatography (HPLC) retention time, percent α-helicity, net charge or isoelectric point (pI); and (c) determining the cell-penetrance and non-specific cell lysis activity of the HSP based on the at least one biophysical property.

In some embodiments, a cell-penetrant hydrocarbon stapled and/or stitched peptide (HSP) includes a staple and/or a stitch at a position of (i and i+3), (i and i+4), or (i and i+7), wherein the positions of the staple and/or stitch are replaced (e.g., substituted or modified) with non-natural amino acids (e.g., amino acids with olefinic side chains). In some embodiments, a cell-penetrant hydrocarbon stapled and/or stitched peptide has a single (i and i+3), (i and i+4), or (i and i+7), staple/stitch, or multiple staples/stitches (e.g., two, three, four, or five).

In some embodiments, the HSP is 6 to 100 amino acids in length. In some cases, the alpha-helical peptide and/or HSP is 6 to 90, 6 to 80, 6 to 70, 6 to 60, 6 to 50, or 6 to 40 amino acids in length (e.g., 10 to 20 amino acids in length, 10 to 30 amino acids in length, or 10 to 40 amino acids in length, 20 to 30 amino acids in length, 20 to 40 amino acids in length, 20 to 50 amino acids in length, or 20 to 60 amino acids in length). In some embodiments, the staple and/or stitch extends the hydrophobic surface beyond the target protein binding surface.

In certain embodiments, the at least one biophysical property of the HSP is isoelectric point (pI). In some embodiments, the pI of the HSP is less than 9.76 (e.g., less than 9.75, less than 9.7, less than 9.6, less than 9.4, less than 9.2, less than 9.0, less than 8.8, less than 8.6, less than 8.5, less than 8.0, less than 7.5, less than 7.0; 7.0, 7.1, 7.2, 7.5, 8.0, 8.1, 8.2, 8.3, 8.4, 8.5, 8.6, 8.7, 8.8, 8.9, 9.0, 9.1 9.2, 9.3, 9.4, 9.5, 9.6, 9.7).

In some embodiments, the at least one biophysical property of the HSP is HPLC retention time. In some embodiments, the HPLC retention time is is 9.57 to 11.2 (e.g., 9.8 minutes, 9.9 minutes, 10 minutes, 10.1 minutes, 10.2 minutes, 10.4 minutes, 10.6 minutes, 10.8 minutes, 11 minutes, 11.2 minutes). In some embodiments, the at least one biophysical property of the HSP is calculated hydrophobicity. In some embodiments, the calculated hydrophobicity is greater than 0.5 (e.g., greater than 0.6, greater than 0.7, greater than 0.8; about 0.5, about 0.6, about 0.7, about 0.8, about 0.9, 0.6, 0.7, 0.8, 0.9). In some embodiments, the at least one biophysical property of the HSP is percent α-helicity. In some embodiments, the percent α-helicity is 61% to 96% (e.g., 61% to 86%). In some embodiments, the at least one biophysical property of the HSP is net charge. In some embodiments, the net charge is +4 to −3 (e.g., +3 to −3, +2 to −2, or +2 to −1).

In some embodiments, the at least one biophysical property of the HSP is isoelectric point (pI) and HPLC retention time. In some embodiments, the at least one biophysical property of the HSP is isoelectric point (pI) and calculated hydrophobicity. In some embodiments, the at least one biophysical property of the HSP is isoelectric point (pI) and percent α-helicity. In some embodiments, the at least one biophysical property of the HSP is isoelectric point (pI) and net charge. In some embodiments, the at least one biophysical property of the HSP is HPLC retention time and calculated hydrophobicity. In some embodiments, the at least one biophysical property of the HSP is HPLC retention time and percent α-helicity. In some embodiments, the at least one biophysical property of the HSP is HPLC retention time and net charge. In some embodiments, the at least one biophysical property of the HSP is HPLC retention time and calculated hydrophobicity. In some embodiments, the at least one biophysical property of the HSP is calculated hydrophobicity and percent α-helicity. In some embodiments, the at least one biophysical property of the HSP is percent α-helicity and net charge. In some embodiments, the at least one biophysical property of the HSP is calculated hydrophobicity and net charge.

In some embodiments, the at least one biophysical property of the HSP is HPLC retention time, pI, and net charge. In some embodiments, the at least one biophysical property of the HSP is HPLC retention time, calculated hydrophobicity, and net charge. In some embodiments, the at least one biophysical property of the HSP is isoelectric point (pI), HPLC retention time, and percent α-helicity. In some embodiments, the at least one biophysical property of the HSP is isoelectric point (pI), HPLC retention time, and calculated hydrophobicity. In some embodiments, the at least one biophysical property of the HSP is isoelectric point (pI), percent α-helicity, and net charge. In some embodiments, the at least one biophysical property of the HSP is isoelectric point (pI), percent α-helicity, and calculated hydrophobicity. In some embodiments, the at least one biophysical property of the HSP is percent α-helicity, HPLC retention and calculated hydrophobicity. In some embodiments, the at least one biophysical property of the HSP is percent α-helicity, HPLC retention, and net charge. In some embodiments, the at least one biophysical property of the HSP is percent α-helicity, calculated hydrophobicity, and net charge. In some embodiments, the at least one biophysical property of the HSP is calculated hydrophobicity, isoelectric point (pI), and net charge.

In some embodiments, the at least one biophysical property of the HSP is HPLC retention time, percent α-helicity, isoelectric point (pI), and net charge. In some embodiments, the at least one biophysical property of the HSP is HPLC retention time, percent α-helicity, isoelectric point (pI), and calculated hydrophobicity. In some embodiments, the at least one biophysical property of the HSP is percent α-helicity, isoelectric point (pI), net charge and calculated hydrophobicity. In some embodiments, the at least one biophysical property of the HSP is HPLC retention time (pH4 or pH7), isoelectric point (pI), net charge, and calculated hydrophobicity. In some embodiments, the at least one biophysical property of the HSP is HPLC retention time (pH4 or pH7), percent α-helicity, calculated hydrophobicity, and net charge.

In some embodiments, the at least one biophysical property of the HSP is HPLC retention time, percent α-helicity, isoelectric point (pI), calculated hydrophobicity, and net charge.

In some embodiments of any of the methods described herein, the pI of the HSP is less than 9.76. In some embodiments of any of the methods described herein, the HSP has a percent α-helicity between 21% to 96%. In some embodiments of any of the methods described herein, the HSP has a HPLC retention time at pH 4 or 7 of 9.56 minutes or greater. In certain embodiments of any of the methods described herein, the net charge of the HSP is +4 to −3 (e.g., +3 to −3, +2 to −2, +2 to −1; +4, +3, +2, +1, 0, −1, −2, or −3).

Also provided herein are pharmaceutical compositions comprising any HSP as described herein. In one aspect, the disclosure features a stapled and/or stitched peptide comprising or consisting of the amino acid sequence set forth in any one of the SEQ ID Nos: 2-38, and 40-49. In certain embodiments, the stapled and/or stitched peptide is less than 75 amino acids in length (e.g., 75 amino acids in length, 65 amino acids in length, 60 amino acids in length, 55 amino acids in length, 50 amino acids in length, 45 amino acids in length, 40 amino acids in length, 35 amino acids in length or 30 amino acids in length). In certain embodiments, the stapled and/or stitched peptides comprise one or more (e.g., 1, 2, 3, 4, 5, 6, 7, 8, 9, or 10) amino acid substitutions within any one of SEQ ID Nos: 2-38 and 40-49. The amino acid substitutions can alter one or more (e.g., 1, 2, 3, 4, or 5) biophysical properties of the stapled and/or stitched peptide. The biophysical properties include: HPLC retention time (pH4 or pH7), calculated hydrophobicity, percent α-helicity, isoelectric point (pI), or net charge. In certain embodiments, one or more (e.g., 1, 2, 3, 4, or 5) of these biophysical parameters fall within the values provided in Table 1.

As used herein, a hydrocarbon-stapled and/or stitched peptide (HSP) having an “overall cellular uptake-facilitating level of hydrophobicity” is an HSP that has an overall level of hydrophobicity that allows the HSP to be taken up by a cell of interest at a level that results in a detectable level of the biological activity of the HSP in the cell. Similarly, as used herein, a hydrocarbon-stapled and/or stitched peptide (HSP) having an “overall cellular uptake-facilitating level of α-helicity” is an HSP that has an overall level of α-helicity that allows the HSP to be taken up by a cell of interest at a level that results in a detectable level of the biological activity of the HSP in the cell. Moreover, as used herein, a hydrocarbon-stapled and/or stitched peptide (HSP) having an “overall cellular uptake-facilitating pI” is an HSP that has a pI that allows the HSP to be taken up by a cell of interest at a level that results in a detectable level of the biological activity of the HSP in the cell. Naturally, methods for measuring the biological activity of HSP will vary greatly according to the relevant activity. Those skilled in the art developing HSPs with particular activities of interest will be familiar with relevant assays and their design.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1A shows exemplary images of mouse embryonic fibroblasts (MEFs) treated with FITC-BIM SAHB_{A1} (bottom left) and stained with Hoechst (top left) and CellMask Deep Red (top right) to monitor the localization of FITC-peptide with respect to cellular architecture (overlay). Images were acquired by IXM at 20× magnification.

FIG. 1B is a graph that shows the total internalized FITC intensity (TIFI) on a per cell basis, monitored upon varying cell density (500 nM peptide, 4 hr, 0% FBS).

FIG. 1C is a graph that shows the total internalized FITC intensity (TIFI) on a per cell basis, monitored upon varying acquisition time (500 nM peptide, 2×10⁴ cells, 0% FBS).

FIG. 1D is a graph that shows the total internalized FITC intensity on a per cell basis, monitored upon varying FITC-peptide dose (2×10⁴ cells, 4 hr, 0% FBS).

FIG. 1E is a graph showing the total internalized FITC intensity on a per cell basis, monitored upon varying percent added FBS (500 nM peptide, 2×10⁴ cells, 4 hr). Error bars represent mean±s.e.m. for experiments performed in duplicate wells with 4 image acquisitions per well. Three biological replicates were performed for each experiment with similar results.

FIG. 2A is a graph that shows the range of TIFI values for BIM BH3 peptides bearing sequentially placed i, i+4 staples (“X” pairs) for MEFs (2×10⁴/well) treated with 500 nM peptides and measured by IXM (20×) at 4 hours. Error bars represent mean±s.d. for experiments performed in duplicate wells with 4 image acquisitions per well. Three biological replicates were performed with similar results (see FIG. 13A). X represents S-pentenyl alanine. SEQ ID Nos. 1-18 are listed sequentially from top to bottom.

(SEQ ID NO: 1)
(IWIAQELRRIGDEFNAYYARR;
(SEQ ID NO: 5)
(IWIXQELXRIGDEFNAYYARR;
(SEQ ID NO: 6)
(IWIAXELRXIGDEFNAYYARR;
(SEQ ID NO: 10)
(IWIAQELRXIGDXFNAYYARR;
(SEQ ID NO: 16)
(IWIAQELRRIGDEFXAYYXARR.

FIG. 2B is a helical wheel projection of the BIM BH3 α-helix, with the hydrophobic interaction face indicated by the dotted surface.

FIG. 2C is a graph that shows single variable plots for TIFI vs. calculated hydrophobicity as assessed by Spearman's rank correlation (p=0.0309). p-value was calculated using the permutation test included in the R package pvrank, and line fitting was performed using a loess smoother.

FIG. 2D is a graph that shows single variable plots for TIFI vs. HPLC retention time (pH 7) as assessed by Spearman's rank correlation (p=0.03). p-value was calculated using the permutation test included in the R package pvrank, and line fitting was performed using a loess smoother.

FIG. 2E is a graph that shows single variable plots for TIFI vs. percent α-helicity as assessed by Spearman's rank correlation (p=0.6733). p-value was calculated using the permutation test included in the R package pvrank, and line fitting was performed using a loess smoother.

FIG. 2F is a graph that shows single variable plots for TIFI vs. net charge at pH 7.4 as assessed by Kruskal-Wallis test (p=0.7497).

FIG. 2G is a graph that shows single variable plots for TIFI vs. pI, as assessed by Spearman's rank correlation (p=0.9962). p-value was calculated using the permutation test included in the R package pvrank, and line fitting was performed using a loess smoother.

FIG. 2H is a tree resulting from recursive partitioning that depicts the influence of principal components (hydrophobicity/HPLC retention time, α-helicity, and pI) on TIFI outcome. Triangles reflect the directionality of parameter values. Retention time and α-helicity are indicated in minutes and percent, respectively.

FIG. 3A is a graph that shows the range of TIFI values for a point mutant library of BIM SAHB_{A1} (BIM SAHB9) peptides, as measured by IXM (20×) in MEFs (2×10⁴ cells/well) treated with 500 nM peptides for 4 hours. Residues that were mutated are underlined. Error bars represent mean±s.d. for experiments performed in duplicate wells with 4 image acquisitions per well. Three biological replicates were performed with similar results. X represents S-pentenyl alanine. SEQ ID NO: 1, SEQ ID NO: 10 and SEQ ID NO: 19-37 are listed sequentially top to bottom. (IWIAQELLXIGDXFNAYYARR (SEQ ID NO:21); (ILIAQELRXIGDXFNAYYARR (SEQ ID NO:23); (IWIAQELRXIGDXFNLYYARR (SEQ ID NO:24); (IWIAQELRXIGDXFNALYARR (SEQ ID NO:25); (IEIAQELRXIGDXFNAYYARR (SEQ ID NO:26); (IWIAQELRXIGDXFNARYARR (SEQ ID NO:29); (IWIAQELDXIGDXFNAYYARR (SEQ ID NO:30); (IWIAQELRXIGDXFNAYYTRR (SEQ ID NO:32); (IRIAQELRXIGDXFNAYYARR (SEQ ID NO:33); (IWIAQELRXIGDXFNEYYARR (SEQ ID NO:34); (IWIAQELRXIGDXFNATYARR (SEQ ID NO:35); (IWIAQELRXIEDXFNAYYARR (SEQ ID NO:36); (IWIAQELRXIGDXVNAYYARR (SEQ ID NO:37).

FIG. 3B is a helical wheel projection of the BIM SAHB_{A1} α-helix, with the hydrophobic interaction face indicated by the dotted surface. Residue positions subjected to differential mutation are partitioned according to their level of TIFI.

FIG. 3C is a graph that shows single variable plots for TIFI vs. calculated hydrophobicity as assessed by Spearman's rank correlation (p=0.4722). p-value was calculated using the permutation test included in the R package pvrank, and line fitting was performed using a loess smoother.

FIG. 3D is a graph that shows single variable plots for TIFI vs. HPLC retention time (pH 7) as assessed by Spearman's rank correlation (p=0.6860). p-value was calculated using the permutation test included in the R package pvrank, and line fitting was performed using a loess smoother.

FIG. 3E is a graph that shows single variable plots for TIFI vs. percent α-helicity as assessed by Spearman's rank correlation (p=0.6350). p-value was calculated using the permutation test included in the R package pvrank, and line fitting was performed using a loess smoother.

FIG. 3F is a graph that shows single variable plots for TIFI vs. net charge at pH 7.4 as assessed by Kruskal-Wallis test (p=0.00658).

FIG. 3G is a graph that shows single variable plots for TIFI vs. pI, as assessed by Spearman's rank correlation (p=0.0658).

FIG. 3H is a tree resulting from recursive partitioning that depicts the influence of principal components (hydrophobicity/HPLC retention time, pI, and α-helicity) on TIFI outcome. Triangles reflect the directionality of parameter values. The point mutants that comprise each data bar are indicated above. Retention time is indicated in minutes.

FIG. 4A is a graph that shows the BCL-X_{L}ΔC binding affinities as determined by fluorescence polarization assay of staple scan libraries (peptide, 25 nm; protein 0.5 nM-1 μM). SEQ ID Nos: 2-18 are listed sequentially from top to bottom.

FIG. 4B is a graph that shows the BCL-X_{L}ΔC binding affinities as determined by fluorescence polarization assay of point mutant FITC-BIM BH3 peptide libraries (peptide, 25 nm; protein 0.5 nM-1 μM). Error bars are mean±s.e.m. for experiments performed in technical duplicate and repeated twice. SEQ ID NO: 10 and SEQ ID Nos: 19-37 are listed sequentially from top to bottom.

FIG. 4C is a graph that shows the effect of stapled peptide treatment (0.3 μM-40 μM) on cell viability of BCL-X_{L}-reconstituted p185⁺Arf^{-/-}Mcl-1^{del} B-ALL cells, as measured by CellTiter Glo assay at 24 hours. Error bars are mean±s.e.m. for experiments performed in technical duplicate and repeated twice. SEQ ID Nos: 2-18 are listed sequentially from top to bottom.

FIG. 4D is a graph that shows the effect of stapled peptide treatment (0.3 μM-40 μM) on cell viability of BCL-X_{L}-reconstituted p185⁺Arf^{-/-}Mcl-1^{del} B-ALL cells, as measured by CellTiter Glo assay at 24 hours. Error bars are mean±s.e.m. for experiments performed in technical duplicate and repeated twice. SEQ ID NO: 10 and SEQ ID Nos: 19-37 are listed sequentially from top to bottom.

FIG. 5A is a graph and helical wheel projection that shows staple scanning that was screened for membrane lytic properties by LDH release assay, performed on BCL-X_{L}-reconstituted p185⁺Arf^{-/-}Mcl-1^{del} B-ALL cells (2×10⁴ cells/well) treated with 10 μM peptide for 30 minutes. Data are normalized based on the response to treatment with 1% Triton X-100 (100% release) and media alone (0% LDH release). Error bars are mean±s.d. for experiments performed in technical duplicate and repeated twice. SEQ ID Nos: 1-18 are listed sequentially from top to bottom.

FIG. 5B is a graph and helical wheel projection that shows point mutant BIM BH3 peptide libraries that were screened for membrane lytic properties by LDH release assay, performed on BCL-X_{L}-reconstituted p185⁺Arf^{-/-}Mcl-1^{del} B-ALL cells (2×10⁴ cells/well) treated with 10 μM peptide for 30 minutes. Data are normalized based on the response to treatment with 1% Triton X-100 (100% release) and media alone (0% LDH release). Error bars are mean±s.d. for experiments performed in technical duplicate and repeated twice. SEQ ID NO: 10 and SEQ ID Nos: 19-37 are listed sequentially from top to bottom.

FIG. 5C is a tree analysis depicting the influence of pI, α-helicity, and HPLC retention time on LDH release outcome. Triangles reflect the directionality of parameter values.

FIG. 6A is a schematic that shows peptide constructs evaluated on the basis of exemplary thresholds for cellular uptake, binding activity, cytotoxicity, and absence of lytic properties. BIM BH3 peptides that contained staples flanking IGD and AYY emerged as the most favorable constructs. Black indicates desirable properties; grey falls below desired threshold. SEQ ID NO: 1-18 are listed sequentially from top to bottom.

FIG. 6B is a schematic that shows peptide constructs with point mutations evaluated. Evaluation of point mutants revealed ideal negative control compounds that retain cellular penetrance without membrane lysis, yet lose target protein binding activity and cytotoxicity by disrupting the interaction surface (e.g., R153D, G156E). SEQ ID NO: 10 and SEQ ID Nos: 19-37 are listed sequentially from top to bottom.

FIG. 7A is a helical wheel projection of the ATSP-7041 α-helix (SEQ ID NO: 38), with the hydrophobic interaction face indicated by the dotted surface.

FIG. 7B is a graph that shows the TIFI value for ATSP-7041 measured by IXM. Error bars represent mean±s.d.

FIG. 7C shows an exemplary image of SAOS-2 cells treated with ATSP-7041. Images were acquired by IXM.

FIG. 7D is a graph that shows the membrane lytic properties of ATSP-7041 by LDH release assay, performed on cells treated with 20 μM peptides. Data are normalized based on the response to treatment with 1% Triton X-100 (100% release) and media alone (0% LDH release). Error bars are mean±s.d.

FIG. 8 is a schematic depicting an exemplary microscopic imaging field. Acquisition points (dark rectangles) in the microscopic field (circle) for measuring total internalized FITC intensity on a per cell basis in 96 well format by IXM at 20× magnification.

FIG. 9A is a graph that shows the optimization of signal-to-noise ratios for fluorescence measurements with respect to DMSO. A custom module (CM) was designed to maximize signal-to-noise detection of FITC-stapled peptides for optimal sensitivity and specificity of internalized peptide measurement. From left to right, the first bar represents no filter (“no filter”), followed by the cumulative addition of a threshold intensity signal requirement of 3 over local background (“+3”), a threshold intensity requirement for objects (cells) having total intensity per cell of 200,000 (“+200,000), and a threshold intensity requirement for objects (cells) displaying an average intensity per cell of 120 (“+120”).

FIG. 9B is a graph that shows the optimization of signal-to-noise ratios for fluorescence measurements with respect to unmodified template peptide. A custom module (CM) was designed to maximize signal-to-noise detection of FITC-stapled peptides for optimal sensitivity and specificity of internalized peptide measurement. From left to right, the first bar represents no filter (“no filter”), followed by the cumulative addition of a threshold intensity signal requirement of 3 over local background (“+3”), a threshold intensity requirement for objects (cells) having total intensity per cell of 200,000 (“+200,000), and a threshold intensity requirement for objects (cells) displaying an average intensity per cell of 120 (“+120”).

FIG. 10 is a graph that shows the effect of cell fixation on TIFI value using live cells treated with DMSO, BIM BH3₁, or BIM SAHB_{A1}.

FIG. 11A is a graph that shows the effect of BIM SAHB_{A1} on plasma membrane integrity. MEFs (2×10⁴ cells/well) were subjected to a serial dilution of BIM SAHB_{A1} for 30 min in media lacking FBS. Cell lyses was measured by LDH release assay. Treatment with 1% Triton X-100 served as the positive control for maximal release. Error bars are mean±s.e.m. for experiments performed in technical duplicate and repeated twice.

FIG. 11B is a graph that shows the effect of BIM SAHBA1 on plasma membrane integrity. MEFs (2×10⁴ cells/well) were subjected to a serial dilution of BIM SAHBA1 for 180 min in media lacking FBS. Cell lyses was measured by LDH release assay. Treatment with 1% Triton X-100 served as the positive control for maximal release. Error bars are mean±s.e.m. for experiments performed in technical duplicate and repeated twice.

FIG. 12 is a dot plot that shows the variation in detected fluorescence signals in DAPI, Cy5 and FITC channels upon treating MEFs with fluorescent stapled peptide. Each dot within a given cluster represents a distinct peptide treatment. The p-value was determined by Kruskal-Wallis test (p<0.0001).

FIG. 13A is a graph that shows TIFI values for cellular uptake of BIM SAHB peptides between biological replicates, as assessed by Spearman's rank correlation (p<0.0001). Each dot represents a distinct stapled peptide from the staple walk library. p-value was calculated using the permutation test included in the R package pvrank, and line fitting was performed using a loess smoother.

FIG. 13B is a graph that shows TIFI values for cellular uptake of BIM SAHB peptides between MEF vs. HeLa cells as assessed by Spearman's rank correlation (p=0.0002). Each dot represents a distinct stapled peptide from the staple walk library. p-value was calculated using the permutation test included in the R package pvrank, and line fitting was performed using a loess smoother.

FIG. 14 is a graph that shows the association between peptide retention time measurements at pH 4 and 7, as assessed by Spearman's rank correlation (p<0.001). Retention times were experimentally determined for each stapled peptide using pH 4 and 7 HPLC buffer conditions. p-value was calculated using the permutation test included in the R package pvrank, and line fitting was performed using a loess smoother.

FIG. 15A is a graph that shows the calculated hydrophobicity vs. experimentally-determined HPLC retention time (pH 7), as assessed by Spearman's rank correlation for BIM BH3 peptides in staple walk libraries (p=0.009). p-value was calculated using the permutation test included in the R package pyrank and line fitting was performed using a loess smoother.

FIG. 15B is a graph that shows the calculated hydrophobicity vs. experimentally-determined HPLC retention time (pH 7), as assessed by Spearman's rank correlation for BIM BH3 peptides in point mutant libraries. p-value was calculated using the permutation test included in the R package pvrank, and line fitting was performed using a loess smoother.

FIG. 16A is a single variable plot that shows TIFI value vs. calculated hydrophobicity of SOS1 peptides (aa 929-944) bearing sequentially placed i, i+7 staples, as assessed by Spearman's rank correlation (p=0.035). p-value was calculated using the permutation test included in the R package pvrank, and line fitting was performed using a loess smoother.

FIG. 16B is a graph that shows the range of TIFI values for MEFs (2×10⁴/well) treated with 500 nM peptides and measured by IXM (20×) at 4 hours. Error bars represent mean±s.d. for experiments performed in duplicate wells with 4 image acquisitions per well. Three biological replicates were performed with similar results. Z represents R-octenyl alanine; X represents S-pentenyl alanine. SEQ ID Nos: 40-49 are listed sequentially from top to bottom.

FIG. 16C is a helical wheel projection of the SOS1 α-helix, with the KRAS-interaction face indicated by the dotted surface.

FIG. 17A is a dot plot that shows the relationship between peptide net charge and isoelectric point for BIM BH3 peptides in staple walk libraries, as determined by Kruskal-Wallis test (p=0.0009).

FIG. 17B is a dot plot that shows the relationship between peptide net charge and isoelectric point for BIM BH3 peptides in point mutant libraries, as determined by Kruskal-Wallis test (p=0.0005).

FIG. 18 is a graph showing cellular LDH release testing results of various SAH-SOS 1 peptides. A staple scanning SOS1 peptide library was screened for membrane lytic properties by LDH release assay, performed on Jurkat T cells (2×10⁴ cells/well) treated with 10 μM peptide for 30 minutes. Data are normalized based on the response to treatment with 1% Triton X-100 (100% release) and media alone (0% LDH release). Error bars are mean±s.d. for experiments performed in technical duplicate and repeated twice. SEQ ID Nos: 39-49 are listed sequentially from top to bottom.

FIG. 19 is a table showing the sequence composition and biophysical parameters of a BIM BH3 staple scanning library. Hydrophobicity was calculated using techniques generally known in the art³⁹, with “X” assigned the hydrophobicity value of Leu. Retention time in minutes reflects the peptide elution time from an HPLC C18 column (Agilent, 1200) run for 20 minutes using a 5-95% water: acetonitrile gradient (10 mM ammonium carbonate). Percent α-helicity of stapled peptides (50 μM, 10% acetonitrile in water) was derived from circular dichroism spectra using the mdeg value at 222 nm, as described³⁴. Net charge was calculated by summing the positive and negative charges of Arg and Asp/Glu, respectively. The isoelectric point (pI) calculation employed the EMBOSS values for pKa. SEQ ID Nos: 1-18 are listed sequentially from top to bottom.

FIG. 20 is a table showing the results of principal component analysis of biophysical parameters impacting the variability of a staple scanning BIM BH3 library. The overall variability in the data can be explained by the cumulative contributions of hydrophobicity/HPLC retention time (component 1), percent α-helicity (component 2), and pI (component 3).

FIG. 21 is a table showing the sequence composition and biophysical parameters of a BIM SAHB_{A1} mutagenesis library. Parameters were calculated or experimentally-derived as described for FIG. 19. SEQ ID Nos: 19-37 are listed sequentially from top to bottom.

FIG. 22 is a table showing the results of principal component analysis of biophysical parameters impacting the variability of a BIM SAHB_{A1} point mutant library. The overall variability in the data can be explained by the cumulative contributions of hydrophobicity/HPLC retention time (component 1), pI (component 2), and percent α-helicity (component 3).

### DETAILED DESCRIPTION

The invention comprises a step-by-step method for the development of stapled and/or stitched peptides with the propensity to be taken up by living cells while avoiding non-specific cell membrane lytic activity (i.e., nonspecific cytotoxicity). We developed a rigorous quantitative approach for measuring intracellular stapled and/or stitched peptides, and generated a comprehensive staple scanning library to prospectively determine what biophysical factors, singly or in combination, correlate with cellular uptake using a recursive partitioning algorithm. We found that the extent of peptide hydrophobicity, as determined by calculation or by measuring HPLC retention time, is the major driver of cellular penetrance, which is also influenced by α-helicity and pI. For the BIM BH3 template, retention times of 9.67-11.26 minutes, coupled with α-helicities of 61-86%, represented the “sweet spot” for cellular uptake. Importantly, we found that stapled and/or stitched peptides with a pI in the 9.76-10.3 range and retention times of 9.78-12.0 minutes, were at greatest risk of inducing nonspecific cellular lysis. Thus, to achieve cellular uptake without membrane disruption, the combination of excessive positive charge and hydrophobicity should be avoided. In generating and analyzing a point mutant library of our lead BIM BH3 stapled peptide, BIM SAHB_{A1}, we observed that cellular uptake can be effectively maintained by lowering the pI to the 8.8-9.34 range while maintaining elevated hydrophobic content (retention times of 9.7-11.2). Thus, in contrast to other classes of cell-penetrating peptides, such as poly-Arg and TAT constructs, positive charge is not a design requirement for generating cell-penetrant stapled and/or stitched peptides. Indeed, the first stapled peptide to emerge as a clinical candidate for intracellular targeting has a net charge of −1⁶.

Consistent with the importance of hydrophobicity in driving the cellular uptake feature of stapled peptides, those BIM BH3 constructs containing staples at the amphipathic boundary were the most penetrant compounds. This suggests that creating an expanded hydrophobic surface on the peptide α-helix is a key design strategy for generating cell permeable stapled peptides (i.e., cell-penetrant hydrocarbon-stapled and/or stitched peptide), as also evidenced by our cellular uptake analysis of SAH-SOS 1 stapled peptides that are composed of a different amino acid sequence and bear an alternate staple type. Indeed, increasing the hydrophobic contact surface may facilitate plasma membrane tropism for cellular import¹⁵. A fortuitous benefit of installing hydrocarbon staples at the boundary of the binding interface is the opportunity for the staple itself to make additional hydrophobic contacts with the protein target. This phenomenon has been observed for a series of cell-penetrant, bioactive stapled peptides as demonstrated by the crystal structures of the MCL-1 SAHBD/MCL-1²⁸, SAH-p53-8/HDM2³³, and ATSP-7041/HDMX⁶ complexes; in each case, staple engagement increased binding affinity without compromising selectivity. Taken together, we found that a stapled peptide design approach that incorporates the described principles of tuning hydrophobicity, α-helicity, and pI provides the best chance for rapidly identifying lead compounds for cellular and clinical application.

Therefore, statistically validated experimental steps include one or more of the following, not necessarily in the specific order listed: (1) all-hydrocarbon staple scan with assessments of hydrophobicity, charge, isoelectric point (pI), and/or α-helicity; (2) mutagenesis scan with alanine, glutamate (or aspartate), and/or arginine (or lysine) to identify tolerated sites for diversification; (3) cell membrane lysis assessment; and (4) tailored biochemical and cellular validation. Insights gleaned from these assays include, e.g., correlation between uptake propensity and (1) hydrophobicity and (2) extension of hydrophobic surface area beyond interaction site by targeted placement of staple and/or a stitch at the amphipathic boundary; and/or correlation between cellular membrane lysis and extent of the combination of (1) hydrophobicity and (2) overall positive charge or elevated p1. Bio-statistical methods that facilitate the correlative analyses include Spearman's and Pearson's correlation testing, principal component analyses, and recursive partitioning. The above-described principles and methods can be applied to rapidly design and validate hydrocarbon-stapled and/or stitched peptides for cellular uptake and preclinical and clinical development. The capacity of appropriately-designed peptide constructs to access the intracellular environment offers new opportunities for targeting and/or modulating a broad array of yet undruggable protein interactions.

A “peptide” or “polypeptide” comprises a polymer of amino acid residues linked together by peptide (amide) bonds. The terms, as used herein, refer to proteins, polypeptides, and peptide of any size, structure, or function. Typically, a peptide or polypeptide will be at least three amino acids long. A peptide or polypeptide may refer to an individual protein or a collection of proteins. In some instances, peptides can include only natural amino acids, although non-natural amino acids (i.e., compounds that do not occur in nature but that can be incorporated into a polypeptide chain) and/or amino acid analogs as are known in the art may alternatively be employed. Also, one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five) of the amino acids in a peptide or polypeptide may be modified, e.g., by the addition of a chemical entity such as a carbohydrate group, a hydroxyl group, a phosphate group, a farnesyl group, an isofarnesyl group, a fatty acid group, a linker for conjugation, functionalization, or other modification, etc. A peptide or polypeptide may also be a single molecule or may be a multi-molecular complex, such as a protein. A peptide or polypeptide may be just a fragment of a naturally occurring protein or peptide. A peptide or polypeptide may be naturally occurring, recombinant, or synthetic, or any combination thereof. “Dipeptide” refers to two covalently linked amino acids.

In some instances, a peptide or polypeptide can include one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five) substitutions in which one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five) amino acid residue(s) is replaced with another amino acid residue. The one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five) amino acid residue(s) can be replaced with any other amino acid residue known in the art, including, e.g., an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Hydrocarbon-Stapled and/or Stitched Peptides

“Peptide stapling” is a term coined from a synthetic methodology wherein two olefin-containing side-chains (e.g., cross-linkable side chains) present in a polypeptide chain (e.g., an alpha-helical peptide chain) are covalently joined (e.g., “stapled together”) using a ring-closing metathesis (RCM) reaction to form a cross-linked ring (Blackwell et al., J. Org. Chem., 66: 5291-5302, 2001; Angew et al., Chem. Int. Ed. 37:3281, 1994). As used herein, the term “peptide stapling” includes the joining of two (e.g., at least one pair of) double bond-containing side-chains, triple bond-containing side-chains, or double bond-containing and triple bond-containing side chain, which may be present in a polypeptide chain, using any number of reaction conditions and/or catalysts to facilitate such a reaction, to provide a singly “stapled” polypeptide (e.g., a hydrocarbon-stapled and/or stitched peptide). The term “multiply stapled” polypeptides refers to those polypeptides containing more than one individual staple, and may contain two, three, or more independent staples of various spacings and compositions. Additionally, the term “peptide stitching,” as used herein, refers to multiple and tandem “stapling” events in a single polypeptide chain to provide a “stitched” (e.g., tandem or multiply stapled) polypeptide, in which two staples, for example, are linked to a common residue. Peptide stitching is disclosed, e.g., in WO 2008121767 and in WO 2010/068684, which are both hereby incorporated by reference in their entireties. In some instances, staples, as used herein, can retain the unsaturated bond or can be reduced (e.g., as mentioned below in the stitching paragraph description). The staples or stitches are positioned at the amphipathic boundary so as to increase the cell-penetrance of the peptides by creating an expanded hydrophobic surface on the peptide α-helix. In some embodiments, a cell-penetrant hydrocarbon stapled and/or stitched peptide (HSP) includes a staple and/or a stitch at a position of (i and i+3), (i and i+4), or (i and i+7), wherein the positions of the staple and/or stitch are replaced (e.g., substituted or modified) with non-natural amino acids (e.g., amino acids with olefinic side chains). In some embodiments, a cell-penetrant hydrocarbon stapled and/or stitched peptide has a single (i and i+3), (i and i+4), or (i and i+7), staple/stitch, or multiple staples/stitches (e.g., two, three, four, or five).

While many peptide staples have all hydrocarbon cross-links, other type of cross-links or staples can be used. For example, triazole-containing (e.g., 1, 4 triazole or 1, 5 triazole) crosslinks can be used (see, e.g., Kawamoto et al. 2012 Journal of Medicinal Chemistry 55:1137; WO 2010/060112).

Stapling of a peptide using all-hydrocarbon cross-link has been shown to help maintain its native conformation and/or secondary structure, particularly under physiologically relevant conditions (see, e.g., Schafmiester et al., J. Am. Chem. Soc., 122:5891-5892, 2000; Walensky et al., Science, 305:1466-1470, 2004).

Stapling the polypeptide herein by an all-hydrocarbon crosslink predisposed to have an alpha-helical secondary structure can improve stability and various pharmacokinetic properties.

Stabilized peptides herein include at least two (e.g., 2, 3, 4, 5, 6) internally cross-linked or stapled amino acids, wherein the at least two amino acids are separated by two (i.e., i, i+3), three (i.e., i, i+4), or six (i.e., i, i+7) amino acids. While at least two amino acids are required to support an internal cross-link (e.g., a staple), additional pairs of internally cross-linked amino acids can be included in a peptide, e.g., to support additional internal cross-links (e.g., staples). For example, peptides can include 1, 2, 3, 4, 5, or more staples.

Alternatively, or in addition, peptides can include three internally cross-linked or stitched amino acids, e.g., yielding two staples arising from a common origin. A peptide stitch includes at least three internally cross-linked amino acids, wherein the middle of the three amino acids (referred to here as the core or central amino acid) forms an internal cross-link (between alpha carbons) with each of the two flanking modified amino acids. The alpha carbon of the core amino acid has side chains that are internal cross-links to the alpha carbons of other amino acids in the peptide, which can be saturated or not saturated. Amino acids cross-linked to the core amino acid can be separated from the core amino acid in either direction by 2, 3, or 6 amino acids (e.g., i, i−3, i, i−4, i, i−7). The number of amino acids on either side of the core (e.g., between the core amino acid and an amino acid cross-linked to the core) can be the same or different.

In some embodiments, peptides herein can include a combination of at least one (e.g., 1, 2, 3, 4, or 5) staple and at least one (e.g., 1, 2, 3, 4, or 5) stitch.

Selection of amino acids for modification (e.g., to support an internal cross-link) can be facilitated by staple scanning. Modification can include, e.g., replacing the existing amino acids with non-natural amino acids (e.g., amino acids with olefinic side chains). The term “staple scan” refers to the synthesis of a library of stapled and/or stitched peptides whereby the location of the i and i+3; i and i+4; and i and i+7 single and multiple staple, or stitches, are positioned sequentially down the length of the peptide sequence, sampling all possible positions, to identify desired or optimal properties and activities for the stapled or stitched constructs. In certain embodiments, the staple(s) and/or stitch(es) are introduced at the amphipathic boundary of the alpha-helix.

Suitable tethers are described herein and, e.g., in US2005/0250680, PCT/US2008/058575, WO 2009/108261, and WO 2010/148335.

Amino acid side chains suitable for use in the peptides disclosed herein are known in the art. For example, suitable amino acid side chains include methyl (as the alpha-amino acid side chain for alanine is methyl), 4-hydroxyphenylmethyl (as the alpha-amino acid side chain for tyrosine is 4-hydroxyphenylmethyl) and thiomethyl (as the alpha-amino acid side chain for cysteine is thiomethyl), etc. A “terminally unsaturated amino acid side chain” refers to an amino acid side chain bearing a terminal unsaturated moiety, such as a substituted or unsubstituted, double bond (e.g., olefinic) or a triple bond (e.g., acetylenic), that participates in crosslinking reaction with other terminal unsaturated moieties in the polypeptide chain. In certain embodiments, a “terminally unsaturated amino acid side chain” is a terminal olefinic amino acid side chain. In certain embodiments, a “terminally unsaturated amino acid side chain” is a terminal acetylenic amino acid side chain. In certain embodiments, the terminal moiety of a “terminally unsaturated amino acid side chain” is not further substituted.

Polypeptides can include more than one crosslink within the polypeptide sequence to either further stabilize the sequence or facilitate the stabilization of longer polypeptide stretches. If the polypeptides are too long to be readily synthesized in one part, independently synthesized, cross-linked peptides can be conjoined by a technique called native chemical ligation (Bang, et al., J. Am. Chem. Soc. 126:1377). Alternately, large peptides are routinely synthesized using a convergent approach whereby fully protected fragments are specifically and sequentially reacted to form the full length desired product, after final deprotection, such as in the industrial synthesis of Fuzeon.

The invention features a modified polypeptide of Formula (I),

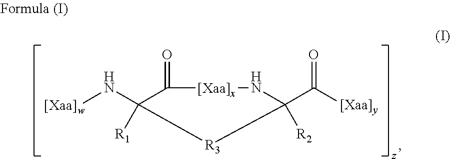

or a pharmaceutically acceptable salt thereof,

wherein;

each R₁ and R₂ are independently H or a C₁ to C₁₀ alkyl, alkenyl, alkynyl, arylalkyl, cycloalkylalkyl, heteroarylalkyl, or heterocyclylalkyl;

each R₃ is alkylene, alkenylene or alkynylene (e.g., a C₆, C₇, or C₁₁ alkenylene) substituted with 1-6 R₄;

each R₄ is, independently —NH₃ or —OH, wherein each —NH₃ is optionally substituted;

wherein each R₃ replaces, relative to the corresponding parent (i.e., unmodified) non-internally cross-linked peptide, the side chains of at least one pair (e.g., one or two pairs) of amino acids separated by 2, 3, or 6 amino acids (i.e., x=2, 3, or 6).

As used above, and elsewhere in the present document, a “corresponding parent (i.e., unmodified) non-internally cross-linked peptide” can be a wild-type peptide, or any of the variants of a wild-type peptide disclosed in the present document, except that such a variant would not include an internal cross-link as described herein.

In the case of Formula I, the following embodiments are among those disclosed.

In cases where x=2 (i.e., i+3 linkage), R₃ can be a C7 alkylene, alkenylene. Where it is an alkenylene there can one or more double bonds. In cases where x=6 (i.e., i+4 linkage), R₃ can be a C₁₁, C₁₂ or C₁₃ alkylene or alkenylene. Where it is an alkenylene there can one or more double bonds. In cases where x=3 (i.e., i+4 linkage), R₃ can be a C₈ alkylene, alkenylene. Where it is an alkenylene, there can one or more double bonds.

In certain instances, the two α,α-disubstituted stereocenters (α carbons) are both in the R configuration or S configuration (e.g., i, i+4 cross-link), or one stereocenter is R and the other is S (e.g., i, i+7 cross-link). Thus, where Formula I is depicted as

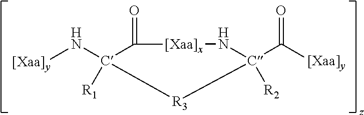

the C′ and C″ disubstituted stereocenters can both be in the R configuration or they can both be in the S configuration, for example when x is 3. When x is 6, the C′ disubstituted stereocenter is in the R configuration and the C″ disubstituted stereocenter is in the S configuration or the C′ disubstituted stereocenter is in the S configuration and the C″ disubstituted stereocenter is in the R configuration. The R₃ double bond may be in the E or Z stereochemical configuration. Similar configurations are possible for the carbons in Formula II corresponding to C′ and C″ in the formula depicted immediately above.

In some instances, the polypeptide includes an amino acid sequence which, in addition to the amino acids side chains that are replaced by a cross-link, have 1, 2, 3, 4 or 5, 6, 7, 8, 9, 10, 11, 12 amino acid changes.

Peptides can contain one or more asymmetric centers and thus occur as racemates and racemic mixtures, single enantiomers, individual diastereomers and diastereomeric mixtures and geometric isomers (e.g. Z or cis and E or trans) of any olefins present. For example, peptides disclosed herein can exist in particular geometric or stereoisomeric forms, including, for example, cis- and trans-isomers, R- and S-enantiomers, diastereomers, (D)-isomers, (L)-isomers, the racemic mixtures thereof, and other mixtures thereof. Enantiomers can be free (e.g., substantially free) of their corresponding enantiomer, and/or may also be optically enriched. “Optically enriched,” as used herein, means that the compound is made up of a significantly greater proportion of one enantiomer. In certain embodiments substantially free means that a composition contains at least about 90% by weight of a preferred enantiomer. In other embodiments the compound is made up of at least about 95%, 98%, or 99% by weight of a preferred enantiomer. Preferred enantiomers may be isolated from racemic mixtures using techniques known in the art, including, but not limited to, for example, chiral high pressure liquid chromatography (HPLC) and the formation and crystallization of chiral salts or prepared by asymmetric syntheses (see, e.g., Jacques, et al, Enantiomers, Racemates and Resolutions (Wiley Interscience, New York, 1981); Wilen, S. H., et al., Tetrahedron 33:2725 (1977); Eliel, EX. Stereochemistry of Carbon Compounds (McGraw-Hill, N Y, 1962); Wilen, S. H. Tables of Resolving Agents and Optical Resolutions p. 268 (EX. Eliel, Ed., Univ. of Notre Dame Press, Notre Dame, Ind. 1972). All such isomeric forms of these compounds are expressly included in the present invention.

Peptides can also be represented in multiple tautomeric forms, in such instances, the invention expressly includes all tautomeric forms of the compounds described herein (e.g., isomers in equilibrium (e.g., keto-enol), wherein alkylation at multiple sites can yield regioisomers), regioisomers, and oxidation products of the compounds disclosed herein (the invention expressly includes all such reaction products). All such isomeric forms of such compounds are included as are all crystal forms.

The term “halo” refers to any radical of fluorine, chlorine, bromine or iodine. The term “alkyl” refers to a hydrocarbon chain that may be a straight chain or branched chain, containing the indicated number of carbon atoms. For example, C₁-C₁₀ indicates that the group may have from 1 to 10 (inclusive) carbon atoms in it. In the absence of any numerical designation, “alkyl” is a chain (straight or branched) having 1 to 20 (inclusive) carbon atoms in it. The term “alkylene” refers to a divalent alkyl (i.e., —R—).

The term “alkenyl” refers to a hydrocarbon chain that may be a straight chain or branched chain having one or more carbon-carbon double bonds in either Z or E geometric configurations. The alkenyl moiety contains the indicated number of carbon atoms. For example, C2-C10 indicates that the group may have from 2 to 10 (inclusive) carbon atoms in it. The term “lower alkenyl” refers to a C2-C8 alkenyl chain. In the absence of any numerical designation, “alkenyl” is a chain (straight or branched) having 2 to 20 (inclusive) carbon atoms in it.

The term “alkynyl” refers to a hydrocarbon chain that may be a straight chain or branched chain having one or more carbon-carbon triple bonds. The alkynyl moiety contains the indicated number of carbon atoms. For example, C₂-C₁₀ indicates that the group may have from 2 to 10 (inclusive) carbon atoms in it. The term “lower alkynyl” refers to a C₂-C₈ alkynyl chain. In the absence of any numerical designation, “alkynyl” is a chain (straight or branched) having 2 to 20 (inclusive) carbon atoms in it.

The term “aryl” refers to a 6-carbon monocyclic or 10-carbon bicyclic aromatic ring system wherein 0, 1, 2, 3, 4, or 5 atoms of each ring may be substituted by a substituent. Examples of aryl groups include phenyl, naphthyl and the like. The term “arylalkyl” or the term “aralkyl” refers to alkyl substituted with an aryl. The term “arylalkoxy” refers to an alkoxy substituted with aryl.

The term “cycloalkyl” as employed herein includes saturated and partially unsaturated cyclic hydrocarbon groups having 3 to 12 carbons, preferably 3 to 8 carbons, and more preferably 3 to 6 carbons, wherein the cycloalkyl group additionally may be optionally substituted. Preferred cycloalkyl groups include, without limitation, cyclopropyl, cyclobutyl, cyclopentyl, cyclopentenyl, cyclohexyl, cyclohexenyl, cyclohexadienyl, cycloheptyl, cycloheptadienyl, cycloheptatrienyl, cyclooctyl, cyclooctenyl, cyclooctadienyl, cyclooctatrienyl, and cyclooctynyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyrrolyl, pyridyl, furyl or furanyl, imidazolyl, 1,2,3-triazolyl, 1,2,4-triazolyl, benzimidazolyl, pyridazyl, pyrimidyl, thiophenyl, quinolinyl, indolyl, thiazolyl, oxazolyl, isoxazolyl, and the like. The term “heteroarylalkyl” or the term “heteroaralkyl” refers to an alkyl substituted with a heteroaryl. The term “heteroarylalkoxy” refers to an alkoxy substituted with heteroaryl.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, aziridinyl, oxiryl, thiiryl, morpholinyl, tetrahydrofuranyl, and the like.

The term “substituents” refers to a group “substituted” on an alkyl, cycloalkyl, aryl, heterocyclyl, or heteroaryl group at any atom of that group. Suitable substituents include, without limitation, halo, hydroxy, mercapto, oxo, nitro, haloalkyl, alkyl, alkaryl, aryl, aralkyl, alkoxy, thioalkoxy, aryloxy, amino, alkoxycarbonyl, amido, carboxy, alkanesulfonyl, alkylcarbonyl, azido, and cyano groups.

Various tethers are envisioned. For example, the tether can be a hydrocarbon tether, and/or can include one or more of an ether, thioether, ester, amine, or amide moiety. In some cases, a naturally occurring amino acid side chain can be incorporated into the tether. For example, a tether can be coupled with a functional group such as the hydroxyl in serine, the thiol in cysteine, the primary amine in lysine, the acid in aspartate or glutamate, or the amide in asparagine or glutamine. Accordingly, it is possible to create a tether using naturally occurring amino acids rather than using a tether that is made by coupling two non-naturally occurring amino acids (i.e. non-natural amino acids). It is also possible to use a single non-naturally occurring amino acid together with a naturally occurring amino acid (i.e non-natural amino acids).

It is further envisioned that the length of the tether can be varied. For instance, a shorter length of tether can be used where it is desirable to provide a relatively high degree of constraint on the secondary alpha-helical structure, whereas, in some instances, it is desirable to provide less constraint on the secondary alpha-helical structure, and thus a longer tether may be desired.

Additionally, while tethers can span from amino acids i to i+3, i to i+4, and i to i+7, the tethers can be synthesized to span any combinations of numbers of amino acids.

In some instances, alpha disubstituted amino acids are used in the polypeptide to improve the stability of the alpha helical secondary structure. However, alpha disubstituted amino acids are not required, and instances using mono-alpha substituents (e.g., in the tethered amino acids) are also envisioned.

The stapled polypeptides can include, e.g., a drug, a toxin, a derivative of polyethylene glycol; a second polypeptide; a carbohydrate, etc. Where a polymer or other agent is linked to the stapled polypeptide it can be desirable for the composition to be substantially homogeneous.

The addition of polyethelene glycol (PEG) molecules can improve the pharmacokinetic and pharmacodynamic properties of the polypeptide. For example, PEGylation can reduce renal clearance and can result in a more stable plasma concentration.

PEG is a water soluble polymer and can be represented as linked to the polypeptide as formula:

XO—(CH₂CH₂O)ₙ—CH₂CH₂—Y

where n is 2 to 10,000 and X is H or a terminal modification, e.g., a C₁₋₄ alkyl; and Y is an amide, carbamate or urea linkage to an amine group (including but not limited to, the epsilon amine of lysine or the N-terminus) of the polypeptide. Y may also be a maleimide linkage to a thiol group (including but not limited to, the thiol group of cysteine). Other methods for linking PEG to a polypeptide, directly or indirectly, are known to those of ordinary skill in the art. The PEG can be linear or branched.

Various forms of PEG including various functionalized derivatives are commercially available.

PEG having degradable linkages in the backbone can be used. For example, PEG can be prepared with ester linkages that are subject to hydrolysis. Conjugates having degradable PEG linkages are described, e.g., in WO 99/34833; WO 99/14259, and U.S. Pat. No. 6,348,558.

In certain embodiments, macromolecular polymer (e.g., PEG) is attached to an agent described herein through an intermediate linker. In certain embodiments, the linker is made up of from 1 to 20 amino acids linked by peptide bonds, wherein the amino acids are selected from the 20 naturally occurring amino acids. Some of these amino acids may be glycosylated, as is well understood by those in the art. In other embodiments, the 1 to 20 amino acids are selected from glycine, alanine, proline, asparagine, glutamine, and lysine. In other embodiments, a linker is made up of a majority of amino acids that are sterically unhindered, such as glycine and alanine. Non-peptide linkers are also possible. For example, alkyl linkers such as —NH(CH2)nC(O)—, wherein n=2-20 can be used. These alkyl linkers may further be substituted by any non-sterically hindering group such as lower alkyl (e.g., C1-C6) lower acyl, halogen (e.g., Cl, Br), CN, NH2, phenyl, etc. U.S. Pat. No. 5,446,090 describes a bifunctional PEG linker and its use in forming conjugates having a peptide at each of the PEG linker termini.

The hydrocarbon-stapled and/or stitched peptides and compositions described herein can be made using any of the methods described throughout the specification. Any of the hydrocarbon-stapled and/or stitched peptides provided herein can have any one or more (e.g., 1, 2, 3, 4, 5) of the biophysical properties as those disclosed in the methods below.

The disclosure features a cell-penetrant hydrocarbon stapled and/or stitched alpha helical peptide that binds a target protein, wherein the hydrocarbon-stapled and/or stitched alpha helical peptide comprises a staple(s) and/or stitch(es) at an amphipathic boundary of the alpha helix of the peptide. In certain embodiments, the staple(s) and/or stitch(es) are at position (i and i+3), (i and i+4), or (i and i+7). This allows an expansion of the hydrophobic surface on the peptide α-helix. In certain instances, the hydrocarbon stapled alpha helical peptide has a calculated hydrophobicity of about 0.5 to about 1.0 (“about” in this context means±0.2). In certain instances, hydrocarbon stapled alpha helical peptide has a HPLC retention time at pH 4.0 or pH 7.0 of 9.67 to 11.26 minutes. In certain instances, the hydrocarbon stapled alpha helical peptide has a calculated hydrophobicity of about 0.5 to about 1.0 and a HPLC retention time at pH 4.0 or pH 7.0 of 9.67 to 11.26 minutes. In some cases, the hydrocarbon stapled alpha helical peptide has an alpha-helicity of about 61% to about 86% (“about” in this context means±4%). In some instances, the hydrocarbon stapled alpha helical peptide has a net charge of +5 to −4. In some instances, the hydrocarbon stapled alpha helical peptide has a net charge of +5 to −3. In some instances, the hydrocarbon stapled alpha helical peptide has a net charge of +4 to −4. In other instances, the hydrocarbon stapled alpha helical peptide has a net charge of +2 to −1. In certain cases, the hydrocarbon stapled alpha helical peptide has an isoelectric point of less than 9.76 (“about” in this context means±0.3). In certain cases, the hydrocarbon stapled alpha helical peptide has an isoelectric point of about 8.8 to about 9.76. In certain cases, the hydrocarbon stapled alpha helical peptide has an isoelectric point of about 5.8 to about 9.76. In certain cases, the hydrocarbon stapled alpha helical peptide has an isoelectric point of about 6.8 to about 9.76.

In certain cases, the hydrocarbon stapled alpha helical peptide has an isoelectric point of about 7.0 to about 9.76. In certain instances, the hydrocarbon stapled alpha helical peptide has a calculated hydrophobicity of about 0.5 to about 1.0; and/or a HPLC retention time at pH 4.0 or pH 7.0 of 9.67 to 11.26 minutes; and/or an isoelectric point of less than 9.76; and/or an alpha-helicity of about 61% to about 86%; and/or a net charge of +2 to −1.

In some instances, the stapled and/or stitched alpha helical peptide comprises one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five)) non-natural amino acids (e.g., S-pentenyl alanine, R-octenyl alanine). In some instances, the stapled and/or stitched alpha helical peptide comprises two S-pentenyl alanines. In other embodiments, the stapled and/or stitched alpha helical peptide comprises two R-octenyl alanines. In certain embodiments, the stapled and/or stitched alpha helical peptide comprises one R-octenyl alanine and one S-pentenyl alanine.

In certain instances, the stapled and/or stitched peptide is about 100 amino acids in length (“about” in this context means±5). In certain instances, the stapled and/or stitched peptide is about 75, 60, 50, 45, 44, 43, 42, 41, 40, 39, 38 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6 or 5 amino acids in length. In certain instances, the stapled and/or stitched peptide is between 6 and 40 amino acids in length (e.g., between 6 and 10 amino acids in length, between 6 and 15 amino acids in length, between 6 and 20 amino acids in length, between 6 and 25 amino acids in length, between 6 and 30 amino acids in length, between 6 and 35 amino acids in length; between 10 and 15 amino acids in length, between 10 and 20 amino acids in length, between 10 and 25 amino acids in length, between 10 and 30 amino acids in length, between 10 and 35 amino acids in length; between 15 and 20 amino acids in length, between 15 and 25 amino acids in length, between 15 and 30 amino acids in length, between 15 and 35 amino acids in length; between 15 and 40 amino acids in length; between 20 and 25 amino acids in length, between 20 and 30 amino acids in length, between 20 and 35 amino acids in length; between 20 and 40 amino acids in length; between 30 and 35 amino acids in length; between 30 and 40 amino acids in length; or between 35 and 40 amino acids in length).

In certain cases, the stapled and/or stitched peptide binds its target protein with a binding affinity of about 1 μM to about 1 nM (e.g., about 1 to about 20 μM, about 1 to about 40 μM, about 1 to about 60 μM, about 1 to about 80 μM, about 1 to about 100 μM, about 1 to about 150 M, about 1 to about 200 μM, about 1 to about 250 μM, about 1 to about 300 μM, about 1 to about 350 μM, about 1 to about 400 μM, about 1 to about 450 μM, about 1 to about 500 μM, about 1 to about 550 μM, about 1 to about 600 μM, about 1 to about 650 μM, about 1 to about 700 μM, about 1 to about 750 μM, about 1 to about 800 μM, about 1 to about 850 μM, about 1 to about 900 μM, about 1 to about 950 μM; about 5 to about 10 μM, about 5 to about 20 μM, about 5 to about 40 μM, about 5 to about 60 μM, about 5 to about 80 μM; about 10 to about 20 μM, about 10 to about 30 μM, about 10 to about 40 μM, about 10 to about 50 μM, about 10 to about 80 μM, about 10 to about 100 μM). In certain cases, the stapled and/or stitched peptide binds its target protein with a binding affinity of about 1 nM to 25 nM (e.g., about 1 nM to about 5 nM, about 1 nM to about 10 nM, about 1 nM to about 20 nM, about 5 nM to about 10 nM, about 5 nM to about 15 nM, about 5 nM to about 20 nM, about 5 nM to about 25 nM, about 10 nM to about 15 nM, about 10 nM to about 20 nM, about 10 nM to about 25 nM, about 15 nM to about 20 nM, about 15 nM to about 25 nM, or about 20 nM to about 25 nM).

In certain embodiments, stapled and/or stitched peptide binds a target protein such as an anti-apoptotic protein (e.g., Bcl-2, Bcl-xL, Bcl-w, MCL-1, BFL-1, and BCL-B) and inhibits its activity. In specific embodiments, the In certain embodiments, the stapled and/or stitched peptide is a BH3 domain of NOXA, BIM, BID, BAK, BOK, BAX, or PUMA. In certain embodiments, stapled and/or stitched peptide is one of the five BIM BH3 peptides that showed improved cellular uptake as described in Example 3. In certain embodiments, stapled and/or stitched peptide is a BIM BH3 peptide. In certain embodiments, the staple and/or stitched peptide is an inhibitor peptide found in cytomegalovirus that binds BAX. In certain embodiments, the stapled and/or stitched peptide is an inhibitor peptide found in eiF4G that binds eiF4E. In certain embodiments, the stapled and/or stitched peptide is a Mediator of RNA polymerase II transcription subunit 11 (med11) peptide. In certain embodiments, the stapled and/or stitched peptide is a FAS ligand peptide that binds to Fas-associated death domain protein (FADD). In certain embodiments, the stapled and/or stitched peptide is a netrin receptor deleted in colorectal cancer (DCC) peptide that binds to the myosin tail homology 4 (MyTH4)-FERM domain. In some embodiments, the stapled and/or stitched peptide is an eiF4A peptide that binds to the tumor suppressor, programmed cell death protein 4 (PDCD4). In some embodiments, the stapled and/or stitched peptide is a RASSF1 peptide that binds to DAXX. In some embodiments, the stapled and/or stitched peptides described herein are cell-penetrant. Cell penetration by the peptides can be easily assessed as described for example in Example 2. The stapled and/or stitched peptides described herein also do not cause lysis of the plasma membrane of the cell or at least exhibit reduced lysis of cells relative to their unstapled and/or unstitched counterpart (i.e., the peptide without the staple(s) and/or stitch(es)). Below are some exemplary assays for evaluating the stapled and/or stitched peptides.

Assays to determine melting temperature (Tₘ): Cross-linked or the unmodified template peptides are dissolved in distilled H₂O or other buffer or solvent (e.g., at a final concentration of 50 μM) and Tₘ is determined by measuring the change in ellipticity over a temperature range (e.g. 4 to 95° C.) on a spectropolarimeter (e.g., Jasco J-710, Aviv) using standard parameters (e.g. wavelength 222 nm; step resolution, 0.5 nm; speed, 20 nm/sec; accumulations, 10; response, 1 sec; bandwidth, 1 nm; temperature increase rate: 1° C./min; path length, 0.1 cm).

In Vitro Protease Resistance Assays:

The amide bond of the peptide backbone is susceptible to hydrolysis by proteases, thereby rendering peptidic compounds vulnerable to rapid degradation in vivo. Peptide helix formation, however, typically buries and/or twists and/or shields the amide backbone and therefore may prevent or substantially retard proteolytic cleavage. The peptidomimetic macrocycles of the present invention may be subjected to in vitro enzymatic proteolysis (e.g., trypsin, chymotrypsin, pepsin) to assess for any change in degradation rate compared to a corresponding uncrosslinked or alternatively stapled polypeptide. For example, the peptidomimetic macrocycle and a corresponding uncrosslinked polypeptide are incubated with trypsin agarose and the reactions quenched at various time points by centrifugation and subsequent HPLC injection to quantitate the residual substrate by ultraviolet absorption at 280 nm. Briefly, the peptidomimetic macrocycle and peptidomimetic precursor (5 mcg) are incubated with trypsin agarose (Pierce) (S/E ˜125) for 0, 10, 20, 90, and 180 minutes. Reactions are quenched by tabletop centrifugation at high speed; remaining substrate in the isolated supernatant is quantified by HPLC-based peak detection at 280 nm. The proteolytic reaction displays first order kinetics and the rate constant, k, is determined from a plot of ln [S] versus time.

Peptidomimetic macrocycles and/or a corresponding uncrosslinked polypeptide can be each incubated with fresh mouse, rat and/or human serum (e.g. 1-2 mL) at 37° C. for, e.g., 0, 1, 2, 4, 8, and 24 hours. Samples of differing macrocycle concentration may be prepared by serial dilution with serum. To determine the level of intact compound, the following procedure may be used: The samples are extracted, for example, by transferring 100 μL of sera to 2 ml centrifuge tubes followed by the addition of 10 μL of 50% formic acid and 500 μL acetonitrile and centrifugation at 14,000 RPM for 10 min at 4+/−2° C. The supernatants are then transferred to fresh 2 ml tubes and evaporated on Turbovap under N₂<10 psi, 37° C. The samples are reconstituted in 100 μL of 50:50 acetonitrile:water and submitted to LC-MS/MS analysis. Equivalent or similar procedures for testing ex vivo stability are known and may be used to determine stability of macrocycles in serum.

In Vivo Protease Resistance Assays:

A key benefit of peptide stapling is the translation of in vitro protease resistance into markedly improved pharmacokinetics in vivo.

Methods of Making a Hydrocarbon-Stapled and/or Stitched Peptide

Methods of synthesizing the compounds of the described herein (e.g., hydrocarbon-stapled and/or stitched peptide) are known in the art. Nevertheless, the following exemplary method may be used. It will be appreciated that the various steps may be performed in an alternate sequence or order to give the desired compounds. Synthetic chemistry transformations and protecting group methodologies (protection and deprotection) useful in synthesizing the compounds described herein are known in the art and include, e.g., those such as described in R. Larock, Comprehensive Organic Transformations, VCH Publishers (1989); T. W. Greene and P. G. M. Wuts, Protective Groups in Organic Synthesis, 3d. Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The peptides of this invention can be made by chemical synthesis methods, which are well known to the ordinarily skilled artisan. See, e.g., Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the α-NH2 protected by either t-Boc or Fmoc chemistry using side chain protected amino acids on, for example, an Applied Biosystems Peptide Synthesizer Model 430A or 431.

One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Longer peptides could be made by conjoining individual synthetic peptides using native chemical ligation. Alternatively, the longer synthetic peptides can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

The peptides can be made in a high-throughput, combinatorial fashion, e.g., using a high-throughput multiple channel combinatorial synthesizer available from Advanced Chemtech.

Peptide bonds can be replaced, e.g., to increase physiological stability of the peptide, by: a retro-inverso bonds (C(O)—NH); a reduced amide bond (NH—CH₂); a thiomethylene bond (S—CH₂ or CH₂—S); an oxomethylene bond (O—CH₂ or CH₂—O); an ethylene bond (CH₂—CH₂); a thioamide bond (C(S)—NH); a trans-olefin bond (CH═CH); a fluoro substituted trans-olefin bond (CF═CH); a ketomethylene bond (C(O)—CHR) or CHR—C(O) wherein R is H or CH₃; and a fluoro-ketomethylene bond (C(O)—CFR or CFR—C(O) wherein R is H or F or CH₃.

The polypeptides can be further modified by: acetylation, amidation, biotinylation, cinnamoylation, farnesylation, fluoresceination, formylation, myristoylation, palmitoylation, phosphorylation (Ser, Tyr or Thr), stearoylation, succinylation and sulfurylation. As indicated above, peptides can be conjugated to, for example, polyethylene glycol (PEG); alkyl groups (e.g., C₁-C₂₀ straight or branched alkyl groups); fatty acid radicals; and combinations thereof.

α,α-disubstituted non-natural amino acids containing olefinic side chains of varying length can be synthesized by known methods (see, e.g., Williams et al. J. Am. Chem. Soc., 113:9276, 1991; Schafmeister et al., J. Am. Chem Soc., 122:5891, 2000; and Bird et al., Methods Enzymol., 446:369, 2008; Bird et al, Current Protocols in Chemical Biology, 2011).

For peptides where an i linked to i+7 staple is used (two turns of the helix stabilized), either: a) one S5 amino acid and one R₈ is used or b) one S8 amino acid and one R₅ amino acid is used. R₈ is synthesized using the same route, except that the starting chiral auxillary confers the R-alkyl-stereoisomer. Also, 8-iodooctene is used in place of 5-iodopentene. Inhibitors are synthesized on a solid support using solid-phase peptide synthesis (SPPS) on MBHA resin (see, e.g., WO 2010/148335).

Fmoc-protected α-amino acids (other than the olefinic amino acids Fmoc-S₅—OH, Fmoc-R₈—OH, Fmoc-R₈—OH, Fmoc-S₈—OH and Fmoc-R₈—OH), 2-(6-chloro-1-H-benzotriazole-1-yl)-1,1,3,3-tetramethylaminium hexafluorophosphate (HCTU), and Rink Amide MBHA are commercially available from, e.g., Novabiochem (San Diego, Calif.). Dimethylformamide (DMF), N-methyl-2-pyrrolidinone (NMP), N,N-diisopropylethylamine (DIEA), trifluoroacetic acid (TFA), 1,2-dichloroethane (DCE), fluorescein isothiocyanate (FITC), and piperidine are commercially available from, e.g., Sigma-Aldrich. Olefinic amino acid synthesis is reported in the art (Williams et al., Org. Synth., 80:3, 2003).

Again, methods suitable for obtaining (e.g., synthesizing), stapling, and purifying the peptides disclosed herein are also known in the art (see, e.g., Bird et. al., Methods in Enzymol., 446:369-386 (2008); Bird et al, Current Protocols in Chemical Biology, 2011; Walensky et al., Science, 305:1466-1470 (2004); Schafmeister et al., J. Am. Chem. Soc., 122:5891-5892 (2000); U.S. patent application Ser. No. 12/525,123, filed Mar. 18, 2010; and U.S. Pat. No. 7,723,468, issued May 25, 2010, each of which are hereby incorporated by reference in their entirety).

In some embodiments, the peptides are substantially free of non-stapled and/or non-stitched peptide contaminants or are isolated. Methods for purifying peptides include, for example, synthesizing the peptide on a solid-phase support. Following cyclization, the solid-phase support may be isolated and suspended in a solution of a solvent such as DMSO, DMSO/dichloromethane mixture, or DMSO/NMP mixture. The DMSO/dichloromethane or DMSO/NMP mixture may comprise about 30%, 40%, 50% or 60% DMSO. In a specific embodiment, a 50%/50% DMSO/NMP solution is used. The solution may be incubated for a period of 1, 6, 12 or 24 hours, following which the resin may be washed, for example with dichloromethane or NMP. In one embodiment, the resin is washed with NMP. Shaking and bubbling an inert gas into the solution may be performed.

In some instances, the stapled and/or stitched alpha helical peptide comprises one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five)1, 2, 3, 4, 5) non-natural amino acids (e.g., S-pentenyl alanine, R-octenyl alanine). In some instances, the stapled and/or stitched alpha helical peptide comprises two S-pentenyl alanines. In other embodiments, the stapled and/or stitched alpha helical peptide comprises two R-octenyl alanines. In certain embodiments, the stapled and/or stitched alpha helical peptide comprises one R-octenyl alanine and one S-pentenyl alanine.

In certain instances, the stapled and/or stitched peptide is about 100 amino acids in length (“about” in this context means±5). In certain instances, the stapled and/or stitched peptide is about 75, 60, 50, 45, 44, 43, 42, 41, 40, 39, 38 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6 or 5 amino acids in length. In certain instances, the stapled and/or stitched peptide is between 6 and 40 amino acids in length (e.g., between 6 and 10 amino acids in length, between 6 and 15 amino acids in length, between 6 and 20 amino acids in length, between 6 and 25 amino acids in length, between 6 and 30 amino acids in length, between 6 and 35 amino acids in length; between 10 and 15 amino acids in length, between 10 and 20 amino acids in length, between 10 and 25 amino acids in length, between 10 and 30 amino acids in length, between 10 and 35 amino acids in length; between 15 and 20 amino acids in length, between 15 and 25 amino acids in length, between 15 and 30 amino acids in length, between 15 and 35 amino acids in length; between 15 and 40 amino acids in length; between 20 and 25 amino acids in length, between 20 and 30 amino acids in length, between 20 and 35 amino acids in length; between 20 and 40 amino acids in length; between 30 and 35 amino acids in length; between 30 and 40 amino acids in length; or between 35 and 40 amino acids in length).

Biophysical properties of the cross-linked polypeptides (e.g., hydrocarbon-stapled and/or stitched peptides) of the invention can be assayed, for example, using the methods described below. In some embodiments, a hydrocarbon-staple and/or stitched peptide is made in accordance with certain biophysical properties (e.g., hydrophobicity, HPLC retention time, percent α-helicity, isoelectric point (pI), net charge). In some embodiments of any of the methods described herein, a hydrocarbon-stapled and/or stitched peptide is cell-penetrant based on the certain biophysical properties as described throughout the specification. In some embodiments of any of the methods described herein, a hydrocarbon-stapled and/or stitched peptide is cell-penetrant and does not exhibit non-specific cell lytic activity (i.e., a hydrocarbon-stapled peptide that is cell-penetrant and that does not cause plasma membrane lysis). In some embodiments of any of the methods described herein, a hydrocarbon-stapled and/or stitched peptide is cell-penetrant and exhibits non-specific cell lytic activity. In some embodiments of any of the methods described herein, a hydrocarbon-stapled and/or stitched peptide is not cell-penetrant.

Methods of Determining Hydrophobicity of a Hydrocarbon-Stapled and/or Stitched Peptide

Various methods are known by those in the art to calculate or to experimentally-determine the hydrophobicity of a peptide or polypeptide (e.g., a hydrocarbon-stapled and/or stitched peptide). Non-limiting examples of such techniques include: hydrophilicity plots; hydropathy plots; Kyte-Doolittle hydrophobicity plots; von Heijne; hydrophobic moment; CCS; Kyle and Eisen scales; and high performance liquid chromatography (HPLC). In some embodiments of any of the methods described herein, the hydrophobicity of a hydrocarbon-stapled and/or stitched peptide can be determined using bioinformatics tools (e.g., ExPASy (www.expasy.org); or HeliQuest (http://heliquest.ipmc.cnrs.fr/cgi-bin/ComputParamsV2.py). In some embodiments, the hydrophobicity of a hydrocarbon-stapled and/or stitched peptide is calculated (e.g., calculated hydrophobicity). In some embodiments, the hydrophobicity of a hydrocarbon-stapled and/or stitched peptide is determined experimentally. In some embodiments, a helical wheel is generated to visually highlight amphipathicity along a helix.

In some embodiments of any of the methods described herein, a hydrocarbon-staple and/or stitched peptide that has a hydrophobicity greater than 0.5, greater than 0.6, greater than 0.7, greater than 0.8, or greater than 0.9 is cell-penetrant. In some embodiments, a hydrocarbon-stapled and/or stitched peptide that has a hydrophobicity of about 0.5 to about 0.7, about 0.5 to about 0.6, about 0.6 to about 0.7, about 0.6 to about 0.8, about 0.7 to 0.8, about 0.7 to 0.9 is cell-penetrant.

In some embodiments of any of the methods described herein, hydrophobicity of a hydrocarbon-stapled and/or stitched peptide is determined by HPLC retention time at pH 4.

In some embodiments of any of the methods described herein, hydrophobicity of a hydrocarbon-stapled and/or stitched peptide is determined by HPLC retention time at pH 7. In some embodiments of any of the methods described herein, a hydrocarbon-stapled and/or stitched peptide that has a HPLC retention time at pH 4 or 7 that is equal to 9.5 minutes or greater, equal to 9.56 minutes or greater, equal to 9.6 minutes or greater, equal to 9.7 minutes or greater, equal to 9.8 minutes or greater, equal to 9.9 minutes or greater, equal to 10.0 minutes or greater, equal to 10.1 minutes or greater, equal to 10.2 minutes or greater, equal to 10.3 minutes or greater, equal to 10.4 minutes or greater, equal to 10.5 minutes or greater, equal to 10.6 minutes or greater, equal to 10.7 minutes or greater, equal to 10.8 minutes or greater, equal to 10.9 minutes or greater, equal to 11.0 minutes or greater, equal to 11.1 minutes or greater, equal to 11.2 minutes or greater, equal to 11.4 minutes or greater, equal to 11.6 minutes or greater, equal to 11.8 minutes or greater, equal to 12.0 minutes or greater, equal to 12.1 minutes or greater, equal to 12.2 minutes or greater, equal to 12.3 minutes or greater, equal to 12.4 minutes or greater, equal to 12.5 minutes or greater; about 9.5 minutes to about 12.5 minutes, about 9.5 minutes to about 12.0 minutes, about 9.5 minutes to about 11.5 minutes, about 9.5 minutes to about 11.2 minutes, about 9.6 minutes to about 11.2 minutes, about 9.7 minutes to about 11.2 minutes, about 9.8 minutes to about 11.2 minutes, about 9.9 minutes to about 11.2 minutes, about 10.0 minutes to about 11.2 minutes, about 10.1 minutes to about 11.2 minutes, about 10.2 minutes to about 11.2 minutes, about 10.3 minutes to about 11.2 minutes, about 10.3 minutes to about 11.2 minutes, about 10.4 minutes to about 11.2 minutes, about 10.5 minutes to about 11.2 minutes, about 10.6 minutes to about 11.2 minutes, about 10.6 minutes to about 11.2 minutes, about 10.7 minutes to about 11.2 minutes, about 10.8 minutes to about 11.2 minutes, about 10.9 minutes to about 11.2 minutes, about 11 minutes to about 11.2 minutes, about 9.5 minutes to about 10 minutes, about 9.5 minutes to about 10.5 minutes, about 9.5 minutes to about 11 minutes, about 9.56 minutes to about 10 minutes, about 9.56 minutes to about 10.5 minutes, about 9.56 minutes to about 11 minutes, or about 9.56 minutes to about 11.2 minutes; and is, generally, cell-penetrant. In other embodiments, the HSP has a HPLC retention time at pH 7 or pH 4 of 9.2 minutes, 9.3 minutes, 9.4 minutes, 9.5 minutes, 9.6 minutes, 9.7 minutes, 9.8 minutes, 9.9 minutes, 10 minutes, 10.1, minutes, 10.2 minutes, 10.3 minutes, 10.4 minutes, 10.5 minutes, 10.6 minutes, 10.7 minutes 10.8 minutes, 10.9 minutes, 11 minutes, 11.1 minutes, or 11.2 minutes.

In some embodiments of any of the methods described herein, a hydrocarbon-stapled and/or stitched peptide that has a HPLC retention time at pH 4 or 7 of less than 9.56 minutes, less than 9.5 minutes, less than 9.4 minutes, less than 9.3 minutes, less than 9.2 minutes, less than 9.1 minutes, less than 9.0 minutes, less than 8.9 minutes less than 8.8 minutes, less than 8.7 minutes, about 1 to about 4 minutes, about 1 to about 5 minutes, about 1 to about 6 minutes, about 1 to about 7 minutes, about 1 to about 8 minutes, about 1 to about 9 minutes, about 1 to about 9.56 minutes, about 2 to about 4 minutes, about 2 to about 6 minutes, about 2 to about 8 minutes, about 2 to about 9.56 minutes, about 3 to about 4 minutes, about 3 to about 5 minutes, about 3 to about 6 minutes, about 3 to about 8 minutes, about 3 to about 9.56 minutes, about 4 to about 5 minutes, about 4 to about 6 minutes, about 4 to about 8 minutes, about 4 to about 9.56 minutes, about 5 to about 6 minutes, about 5 to about 8 minutes, about 5 to about 9.56 minutes, about 6 to about 8 minutes, about 6 to about 9.56 minutes, about 7 to about 8 minutes, about 7 to about 9.56 minutes, or about 8 to about 9.56 minutes, is not cell-penetrant.

In some embodiments of any of the methods described herein, altering (e.g., increasing) the hydrophobicity of a hydrocarbon-stapled and/or stitched peptide results in placing a staple and/or a stitch at the amphipathic boundary (i.e., at the hydrophobic/hydrophilic boundary). In some embodiments of any of the methods described herein, placing a staple and/or a stitch at the amphipathic boundary extends the hydrophobic surface beyond the hydrophobic portion of the amphipathic helix and/or the target protein binding surface. In some embodiments of any of the methods described herein, placing a staple and/or a stitch at the amphipathic boundary increases the hydrophobic contact surface to facilitate plasma membrane tropism for cellular import. In some embodiments of any of the methods described herein, placing a staple and/or a stitch at the amphipathic boundary provides additional hydrophobic contacts with the protein target. In some embodiments of any of the methods described herein, a hydrocarbon-stapled and/or stitched peptide is determined to be cell-penetrant and/or not lytic, or non-cell-penetrant. As used herein the term “cell-penetrant” or “cellular uptake” refers to the ability of a hydrocarbon-stapled and/or stitched peptide to be internalized by the host cell. As used herein the terms “host cell” and “cell” are interchangeable. In some embodiments of any of the methods described herein, a cell-penetrant hydrocarbon-stapled and/or stitched peptide can be internalized by any cell, e.g., a eukaryotic cell (e.g., a mammalian cell, a rodent cell, a non-human primate cell, a human cell, a fungal cell, an insect cell, or a plant cell). In some embodiments, the mammalian cell is a cancer cell or an immune cell. In some embodiments, the mammalian cell is a nerve cell, an adipocyte, a blood cell, a muscle cell, or a skin cell. In some embodiments, the mammalian cell is an exocrine secretory epithelial cell. In some embodiments, the mammalian cell is a hormone-secreting cell. In some embodiments, the mammalian cell is a keratinizing epithelial cell. In some embodiments, the mammalian cell is an epithelial cell. In some embodiments, the mammalian cell is a sensory transducer cell. In some embodiments, the mammalian cell is an autonomic neuron cell. In some embodiments, the mammalian cell is a send organ and/or peripheral neuron supporting cell. In some embodiments, the mammalian cell is a central nervous system neuron or a glial cell. In some embodiments, the mammalian cell is a lens cell. In some embodiments, the mammalian cell is a metabolic and storage cell. In some embodiments, the mammalian cell is a kidney cell. In some embodiments, the mammalian cell is a contractile cell. In some embodiments, the mammalian cell is a germ cell (e.g., an oocyte, a spermatid, or a spermatocyte). In some embodiments, the mammalian cell is a nurse cell. In some embodiments, the mammalian cell is an interstitial cell.

In some embodiments, the mammalian cell is a cell representative of a disease or a diseased state. In some embodiments, the disease is coronary artery disease (e.g., ischemic heart disease), stroke, or chronic obstructive pulmonary disease (COPD). In some embodiments, the disease is a lower respiratory infection. In some embodiments, the disease is a cancer (e.g., trachea, bronchus and lung cancer (e.g., non-small cell lung cancer)). In some embodiments, the disease is a human immunodeficiency virus (HIV) infection. In some embodiments, the disease is acquired immune deficiency syndrome (AIDS). In some embodiments, the disease is a diarrheal disease (e.g., Crohn's disease, or ulcerative colitis). In some embodiments, the disease is diabetes mellitus. In some embodiments, the mammalian cell is a patient-derived cancer cell. In some embodiments, the mammalian cell is a patient-derived immune cell.

Methods of Determining Percent α-Helicity of a Hydrocarbon-Staple and/or Stitched Peptide

The alpha-helicity of a peptide of interest can be determined by any method known in the art. In some cases, circular dichroism or nuclear magnetic resonance spectroscopy (NMR) may be employed. Peptides are dissolved in an aqueous solution (e.g., 5 mM potassium phosphate solution at pH 7, or distilled H₂O, to concentrations of 25-50 μM). Circular dichroism (CD) spectra are obtained on a spectropolarimeter (e.g., Jasco J-710, Aviv) using standard measurement parameters (e.g., temperature, 20° C.; wavelength, 190-260 nm; step resolution, 0.5 nm; speed, 20 nm/sec; accumulations, 10; response, 1 sec; bandwidth, 1 nm; path length, 0.1 cm). The α-helical content of each peptide is calculated by dividing the mean residue ellipticity by the reported value for a model helical decapeptide (Yang et al., Methods Enzymol. 130:208 (1986)).

In some embodiments of any of the methods described herein, a hydrocarbon-stapled and/or stitched peptide that has a percent α-helicity of 61% to 86%, 62 to 86%, 63% to 86%, 64 to 86%, 65% to 86%, 66% to 86%, 67% to 86%, 68% to 86%, 69% to 86%, 70% to 86%, 71% to 86%, 72% to 86%, 73% to 86%, 74% to 86%, 75% to 86%, 76% to 86%, 77% to 86%, 78% to 86%, 79% to 86%, 80% to 86%, 81% to 87%, 82% to 86%, 83% to 86%, 84% to 86%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, or 86%, is cell-penetrant. In some embodiments, the HSP has a percent α-helicity of 40% to 90%, 40% to 85%, 40% to 80%, 40% to 75%, 40% to 70%, 40% to 65%, 40% to 60%, 40% to 55%, 40% to 50%, 45% to 90%, 45% to 85%, 45% to 80%, 45% to 75%, 45% to 70%, 45% to 65%, 45% to 60%, 45% to 55%, 45% to 50%, 50% to 90%, 50% to 85%, 50% to 80%, 50% to 75%, 50% to 70%, 50% to 65%, 50% to 60%, 50% to 55%, 55% to 90%, 55% to 85%, 55% to 80%, 55% to 75%, 55% to 70%, 55% to 65%, 55% to 60%, 60% to 90%, 60% to 85%, 60% to 80%, 60% to 75%, 60% to 70%, 60% to 65%, 65% to 90%, 65% to 85%, 65% to 80%, 65% to 75%, 65% to 70%, 70% to 90%, 70% to 85%, 70% to 80%, 70% to 75%, 75% to 90%, 75% to 85%, 75% to 80%, 80% to 90%, 80% to 85%, 85% to 90%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, or 90%.

In some embodiments, of any of the methods described herein, a hydrocarbon-stapled and/or stitched peptide that has a percent α-helicity of less than 20%, less than 18%, less than 16%, less than 14%, less than 12%, less than 10%, less than 8%, less than 6%, less than 4%, less than 2%, less than 1%; and is not cell-penetrant.

Methods of Determining Cellular Penetrance of a Hydrocarbon-Stapled and/or Stitched Peptide

The cellular penetrance of a peptide of interest can be assessed by any method known in the art. For example, ImageXpress microscopy analysis may be employed as described in Example 1. The total internalized FITC intensity (TIFI) can be used as a way to determine cellular uptake of a hydrocarbon-stapled and/or stitched peptide by a cell (e.g., any cell described herein). Methods of determining TIFI are described in detail in Examples 2 and 3 of the instant specification. Briefly, a custom module (CM) was created to develop a rigorous quantitation platform for measuring cellular uptake of hydrocarbon-stapled and/or stitched peptides derivatized with a FITC fluorophore at the N-terminus. The CM was developed based on (1) defining the parameters of cellular uptake (e.g., excluding extracellular aggregates and auto-fluorescent debris), (2) defining a pixel boundary to avoid quantitation of extracellular, membrane-adherent peptides, (3) excluding out-of-focus fluorescence from the FITC signal outliers. Once the CM parameters are set, images can be acquired at 20× and 40× magnification for per cell basis fluorescence, and four distinct central locations of the cellular field are visualized. Next signal-to-noise ratios are maximized to ensure optimal sensitivity and specificity of internal FITC-peptide signal. In certain instances, the following parameters are used to maximize sensitivity and specificity of the detection method: cell density: 2×10⁴; acquisition time: 4 hours: dosing of peptide: 500 nM; and 0% FBS.

In some embodiments, a hydrocarbon-stapled and/or stitched peptide that is cell-penetrant has a TIFI that is greater than 0.5×10⁶, greater than 0.6×10⁶, greater than 0.7×10⁶, greater than 0.8×10⁶, greater than 0.9×10⁶, greater than 1.0×10⁶, greater than 1.2×10⁶, greater than 1.5×10⁶, greater than 1.75×10⁶, greater than 2.0×10⁶, greater than 2.25×10⁶, greater than 2.5×10⁶, greater than 2.75×10⁶, greater than 3.5×10⁶, greater than 4.0×10⁶, greater than 4.5×10⁶, greater than 5.0×10⁶, greater than 5.5×10⁶, greater than 6.0×10⁶, about 0.5×10⁶ to about 3.0×10⁶, 0.5×10⁶ to about 6×10⁶, about 1×10⁶ to about 3×10⁶, about 1×10⁶ to about 6×10⁶, or about 3×10⁶ to about 6×10⁶; 0.5×10⁶, 1.0×10⁶, 1.2×10⁶, 1.3×10⁶,1.5×10⁶, 2.0×10⁶, 2.5×10⁶, 3.0×10⁶, 3.5×10⁶, 4.0×10⁶, 4.5×10⁶, 5.0×10⁶, or 6.0×10⁶.

In some embodiments, a hydrocarbon-stapled and/or stitched peptide that has a TIFI that is less than 0.5×10⁶, less than 0.4×10⁶, less than 0.3×10⁶, less than 0.2×10⁶, or less than 0.25×10⁶, less than 0.5×10⁶ (e.g., less than 4×10⁵, less than 2.0×10⁵, less than 1×10⁵, less than 0.5×10⁵, less than 4×10⁴, less than 2.0×10⁴, less than 1×10⁴, less than 0.5×10⁴, less than 4×10³, less than 2.0×10³, less than 1×10³, less than 0.5×10³; about 0 to about 0.5×10⁶, about 0 to about 1×10³, or about 0 to about 1×10⁴) is not cell-penetrant.

Methods of Determining Isoelectric Point of a Hydrocarbon Stapled and/or Stitched Peptide

Various methods are known by those in the art to calculate or to experimentally-determine the isoelectric point (pI) of a peptide or polypeptide (e.g., a hydrocarbon-stapled and/or stitched peptide). As used herein, the term “isoelectric point” refers to the pH at which a peptide or polypeptide (e.g., a hydrocarbon-stapled and/or stitched peptide) is neutral (e.g., the pH at which a peptide or polypeptide does not migrate in an electric field). Non-limiting examples of techniques to determine pI include: isoelectric focusing, and bioinformatics tools and/or platforms.

In some embodiments of any of the methods described herein, a hydrocarbon-stapled and/or stitched peptide that has a pI less than 9.76 is cell-penetrant. In certain embodiments, the hydrocarbon-stapled and/or stitched peptide that has a pI less than 9.7, less than 9.6, less than 9.5, less than 9.4, less than 9.3, less than 9.2, less than 9.1, less than 9.0, less than 8.9, less than 8.8, less than 8.7, less than 8.6, less than 8.5, less than 8.4, less than 8.3, less than 8.0, less than 7.9, less than 7.7, or less than 7.0; 6.5, 6.6, 6.7, 6.8, 6.9, 7.0, 7.1, 7.2, 7.3, 7.4, 7.5 7.6 7.7, 7.8, 7.9, 8.0, 8.1, 8.2, 8.3, 8.4, 8.5, 8.6, 8.7 8.8, 8.9, 9.0, 9.1, 9.2, 9.3, 9.4, 9.5, or 9.6 is cell-penetrant. In other embodiments, a cell-penetrant HSP has a pI of 9.75, 9.74, 9.73, 9.72, 9.71, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, or 6.0. In other embodiments, a cell-penetrant HSP has a pI of 6.0 to 9.75. In other embodiments, a cell-penetrant HSP has a pI of 7.0 to 9.75. In other embodiments, a cell-penetrant HSP has a pI of 8.0 to 9.75.

In some embodiments of any of the methods described herein, a hydrocarbon-stapled and/or stitched peptide that has a pI greater than 9.76 (e.g., 9.76 to 10.30) is not cell-penetrant.

Methods of Optimizing a Cell-Penetrant Hydrocarbon-Stapled and/or Stitched Peptide

Provided herein are methods of refining (or optimizing) biophysical properties of cell-penetrant hydrocarbon-stapled and/or stitched peptides. The disclosure provides methods of improving upon peptides that have desired activities (e.g., cellular uptake and target protein binding but that do not induce membrane lysis (i.e. cellular uptake and target protein binding but that does not exhibit non-specific cell lytic activity). For example, target protein binding affinity of the peptide may be improved and/or the peptide's cell-penetrance may be improved. The methods include the steps of: providing a first alpha-helical HSP that binds a target protein; generating a second HSP that is identical to the first HSP except at one or more (e.g., one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five) amino acid positions, wherein the second HSP binds to the same target as the first HSP, and has at least one altered (e.g., increased or decreased) biophysical property compared to the first HSP; wherein the at least one biophysical property is selected from: hydrophobicity, high performance liquid chromatography (HPLC) retention time, percent α-helicity, net charge, and isoelectric point (pI); and wherein either (i) the HPLC retention time of the second HSP is altered compared to the first HSP, and the second HSP has improved cellular uptake as compared to the first HSP; (ii) the hydrophobicity of the second HSP is altered compared to the first HSP, and the second HSP has improved cellular uptake as compared to the first HSP; (iii) the net charge of the second HSP is altered compared to the first HSP, and the second HSP has improved cellular uptake as compared to the first HSP; or (iv) the percent α-helicity of the second HSP is altered compared to the first HSP, and the second HSP has improved cellular uptake as compared to the first HSP.

In some embodiments, the second HSP has a lower pI as compared to the first HSP, and the second HSP has improved cellular uptake as compared to the first HSP. In some embodiments, the second HSP has a pI of 8.0 to 9.75 (e.g., 8.8 to 9.34, 8.0 to 8.5, 8.0 to 9.0, 8.0 to 9.5, 8.0 to 9.75, 8.5 to 9.0, 8.5 to 9.5, 8.5 to 9.75, 9.0 to 9.5, or 9.0 to 9.75).

In some embodiments, the second HSP has a maintained HPLC retention time as compared to the first HSP, and the second HSP has improved cellular uptake as compared to the first HSP. In some embodiments, the second HSP has an increased HPLC retention time as compared to the first HSP, and the second HSP has improved cellular uptake as compared to the first HSP.

In some embodiments, the second HSP has a HPLC retention time at pH 7 equal to 9.5 minutes or greater, equal to 9.56 minutes or greater, equal to 9.6 minutes or greater, equal to 9.7 minutes or greater, equal to 9.8 minutes or greater, equal to 9.9 minutes or greater, equal to 10.0 minutes or greater, equal to 10.1 minutes or greater, equal to 10.2 minutes or greater, equal to 10.3 minutes or greater, equal to 10.4 minutes or greater, equal to 10.5 minutes or greater, equal to 10.6 minutes or greater, equal to 10.7 minutes or greater, equal to 10.8 minutes or greater, equal to 10.9 minutes or greater, equal to 11.0 minutes or greater, equal to 11.1 minutes or greater; about 9.5 minutes to about 11.2 minutes, about 9.6 minutes to about 11.2 minutes, about 9.7 minutes to about 11.2 minutes, about 9.8 minutes to about 11.2 minutes, about 9.9 minutes to about 11.2 minutes, about 10.0 minutes to about 11.2 minutes, about 10.1 minutes to about 11.2 minutes, about 10.2 minutes to about 11.2 minutes, about 10.3 minutes to about 11.2 minutes, about 10.3 minutes to about 11.2 minutes, about 10.4 minutes to about 11.2 minutes, about 10.5 minutes to about 11.2 minutes, about 10.6 minutes to about 11.2 minutes, about 10.6 minutes to about 11.2 minutes, about 10.7 minutes to about 11.2 minutes, about 10.8 minutes to about 11.2 minutes, about 10.9 minutes to about 11.2 minutes, about 11 minutes to about 11.2 minutes, about 9.5 minutes to about 10 minutes, about 9.5 minutes to about 10.5 minutes, about 9.5 minutes to about 11 minutes, about 9.56 minutes to about 10 minutes, about 9.56 minutes to about 10.5 minutes, about 9.56 minutes to about 11 minutes, or about 9.56 minutes to about 11.2 minutes, and the second HSP has improved cellular uptake as compared to the first HSP.

In some embodiments, the second HSP binds to the same target as the first HSP with the same binding affinity to the target as compared to the first HSP. In some embodiments, the second HSP binds to the same target as the first HSP with greater binding affinity to the target as compared to the first HSP.

In certain embodiments, stapled and/or/stitched peptide binds a target protein such as an anti-apoptotic protein (e.g., Bcl-2, Bcl-xL, Bcl-w, MCL-1, BFL-1, and BCL-B) and inhibits its activity. In specific embodiments, the In certain embodiments, the stapled and/or/stitched peptide is a BH3 domain of NOXA, BIM, BID, BAK, BOK, BAX, or PUMA. In certain embodiments, stapled and/or/stitched peptide is one of the five BIM BH3 peptides that showed improved cellular uptake as described in Example 3. In certain embodiments, stapled and/or/stitched peptide is a BIM BH3 peptide. In certain embodiments, wherein the stapled and/or stitched peptide is a BIM BH3 peptide, the amino acid at position 151 is not a hydrophobic residue (i.e., stapling amino acid substitution or leucine mutation). In certain embodiments, wherein the stapled and/or stitched peptide is a BIM BH3 peptide, a mutation of amino acid at position 147 to Arginine, indicates that the stapled and/or stitched peptide is lytic. In certain embodiments, wherein the stapled and/or stitched peptide is a BIM BH3 peptide, a mutation of amino acid at position 164 to Threonine, indicates that the stapled and/or stitched peptide is lytic.

These methods are based on the modifications of specific amino acid residues of a first hydrocarbon-stapled and/or stitched peptide that may serve as a control and/or reference hydrocarbon-stapled and/or stitched peptide. Specific amino acid residues of a first hydrocarbon-stapled and/or stitched peptide can be modified by various methods, e.g., site-direct mutagenesis or by making a point mutant library. In some embodiments, the at least one point-mutation is a non-synonymous substitution (i.e., at least one point-mutation that results in a codon that encodes for a different amino acid). The at least one point-mutation can result in an amino acid being replaced with any other amino acid residue known in the art, including, e.g., an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In some embodiments, the at least one point-mutation results in an amino acid substitution that does not have a similar side chain as the amino acid at that position in the first hydrocarbon-stapled and/or stitched peptide.

Methods of Making a Cell-Penetrant Hydrocarbon-Stapled and/or Stitched Peptide that does not Exhibit Non-Specific Cell Lytic Activity

Provided herein are methods of selecting a cell-penetrant hydrocarbon-stapled and/or stitched peptide (HSP) that is not non-specific cell lytic that include: (a) providing an alpha helical peptide that binds a target protein; (b) preparing a stapled and/or stitched version of the alpha helical peptide by introducing a staple and/or stitch in the peptide, wherein the staple and/or stitch is located at an amphipathic boundary of the alpha helix; (c) determining the percent α-helicity or isoelectric point (pI); and (c) selecting the HSP as not exhibiting non-specific cell lytic activity, wherein the HSP comprises: (i) an isoelectric point (pI) that is less than 9.76, and (ii) a percent α-helicity that ranges from 21% to 96%.

In some embodiments, the HSP that is not lytic comprises an isoelectric point (pI) that is less than 9.76, less than 9.7, less than 9.6, less than 9.5, less than 9.4, less than 9.3, less than 9.2, less than 9.1, less than 9.0, less than 8.9, less than 8.8, less than 8.7, or less than 8.6 and a percent α-helicity that ranges from 21% to 96%, 21% to 25%, 21% to 30%, 21% to 35%, 21% to 40%, 21% to 45%, 21% to 50%, 21% to 55%, 21% to 60%, 21% to 65%, 21% to 70%, 21% to 75%, 21% to 80%, 21% to 85%, 21% to 90%, 21% to 95%, 25% to 30%, 25% to 35%, 25% to 40%, 25% to 45%, 25% to 50%, 25% to 55%, 25% to 60%, 25% to 65%, 25% to 70%, 25% to 75%, 25% to 80%, 25% to 85% 25% to 90%, 25% to 95%, 25% to 96%, 30% to 35%, 30% to 40%, 30% to 45%, 30% to 50%, 30% to 55%, 30% to 60%, 30% to 65%, 30% to 70%, 30% to 75%, 30% to 80%, 30% to 85%, 30% to 90%, 30% to 95%, 30% to 96%, 35% to 40%, 35% to 45%, 35% to 50%, 35% to 55%, 35% to 60%, 35% to 65%, 35% to 70%, 35% to 75%, 35% to 80%, 35% to 85%, 35% to 90%, 35% to 95%, 35% to 96%, 40% to 45%, 40% to 50%, 40% to 55%, 40% to 60%, 40% to 65%, 40% to 70%, 40% to 75%, 40% to 80%, 40% to 85%, 40% to 90%, 40% to 95%, 40% to 96%, 50% to 55%, 50% to 60%, 50% to 65%, 50% to 70%, 50% to 75%, 50% to 80%, 50% to 85%, 50% to 90%, 50% to 95%, 50% to 96%, 55% to 60%, 55% to 65%, 55% to 70%, 55% to 75%, 55% to 80%, 55% to 85%, 55% to 90%, 55% to 95%, 55% to 96%, 60% to 65%, 60% to 70%, 60% to 75%, 60% to 80%, 60% to 85%, 60% to 90%, 60% to 95%, 60% to 96%, 65% to 70%, 65% to 75%, 65% to 80%, 65% to 85%, 65% to 90%, 65% to 95%, 65% to 96%, 70% to 75%, 70% to 80%, 70% to 85%, 70% to 90%, 70% to 95%, 70% to 96%, 75% to 80%, 75% to 85%, 75% to 90%, 75% to 95%, 75% to 96%, 80% to 85%, 80% to 90%, 80% to 95%, 80% to 96%, 85% to 90%, 85% to 95%, 85% to 96%, 90% to 95%, 90% to 96%; 21%, 22%, 24%, 26%, 28%, 30%, 32%, 33%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, or 90%.

Also provided herein are methods of determining cell-penetrance and non-specific cell lysis activity of a hydrocarbon-stapled and/or stitched peptide (HSP) that include: (a) providing an alpha helical peptide that binds a target protein; preparing a stapled and/or stitched version of the alpha helical peptide by introducing a staple and/or stitch in the peptide, wherein the staple and/or stitch is located at an amphipathic boundary of the alpha helix; (b) determining at least one biophysical property of the HSP; wherein the at least one biophysical property is hydrophobicity, HPLC retention time, percent α-helicity, or isoelectric point (pI); (c) determining the cell-penetrance and non-specific cell lysis activity of the HSP based on the at least one biophysical property.

In some embodiments, the at least one biophysical property of the HSP is isoelectric point (pI). In some embodiments, the at least one biophysical property of the HSP is isoelectric point (pI) and one additional biophysical property selected from the group: HPLC retention time and percent α-helicity. In some embodiments, the pI of the HSP is less than 9.76 (or any of the ranges provided herein) and the additional biophysical property is percent α-helicity, and the HSP has a percent α-helicity between 21% to 96% (or any of the ranges provided herein). In some embodiments, the pI of the HSP is less than 9.76 and the additional biophysical property is HPLC retention time, and the HPLC retention time is 9.57 to 11.2 (e.g., 9.8 minutes, 9.9 minutes, 10 minutes, 10.1 minutes, 10.2 minutes, 10.4 minutes, 10.6 minutes, 10.8 minutes, 11 minutes, or 11.2 minutes), and the peptide does not exhibit non-specific cell lytic activity. In some embodiments, the pI of the HSP is greater than 9.76 (e.g., 9.76 to 10.30) and the additional biophysical property is HPLC retention time, and the HPLC retention time is less than 9.77 or less than 8.7, and the peptide does not exhibit non-specific cell lytic activity.

In some embodiments, the pI of the HSP is greater than 9.76, greater than 9.8, greater than 9.9, greater than 9.9, greater than 10.0, greater than 10.1, greater than 10.2, or equal to 10.3; and the additional biophysical property is HPLC retention time, and the HSP has a HPLC retention time at pH 7 that is about 9.78 minutes to about 12 minutes, about 9.8 minutes to about 12 minutes, about 9.9 minutes to about 12 minutes, about 10.0 minutes to about 12 minutes, about 10.1 minutes to about 12 minutes, about 10.2 minutes to about 12 minutes, about 10.3 minutes to about 12 minutes, about 10.3 minutes to about 12 minutes, about 10.4 minutes to about 12 minutes, about 10.5 minutes to about 12 minutes, about 10.6 minutes to about 12 minutes, about 10.6 minutes to about 12 minutes, about 10.7 minutes to about 12 minutes, about 10.8 minutes to about 12 minutes, about 10.9 minutes to about 12 minutes, about 11.0 minutes to about 12 minutes, about 11.2 to about 12 minutes, about 11.4 to about 12 minutes, about 11.6 to about 12 minutes, about 11.8 to about 12 minutes, and exhibits non-specific cell lytic activity.

In some embodiments, the HSP has a net charge of +4 to −3. In other embodiments, the HSP has a net charge of +3 to −3. In certain embodiments, the HSP has a net charge of +2 to −2. In some embodiments, the HSP has a net charge of +2 to −1 (e.g., a net charge of +2, a net charge of +1, a net charge of 0, a net charge of −1).

In some embodiments of any of the methods described herein, one or more (e.g., one, two, three, four, or five) biophysical parameters can be determined to determine the cell-penetrance and non-specific cell lysis activity of a HSP. For example, the isoelectric point (pI) and HPLC retention time of a HSP can be determined to determine the cell-penetrance and non-specific cell lysis activity. In certain embodiments, the isoelectric point (pI) and calculated hydrophobicity of a HSP can be determined to determine the cell-penetrance and non-specific cell lysis activity. In certain embodiments, the isoelectric point (pI) and percent α-helicity of a HSP can be determined to determine the cell-penetrance and non-specific cell lysis activity. In certain embodiments, the isoelectric point (pI) and net charge of a HSP can be determined to determine the cell-penetrance and non-specific cell lysis activity. In certain embodiments, the a HPLC retention time and calculated hydrophobicity of a HSP can be determined to determine the cell-penetrance and non-specific cell lysis activity. In certain embodiments, the HPLC retention time and percent α-helicity of a HSP can be determined to determine the cell-penetrance and non-specific cell lysis activity. In certain embodiments, the HPLC retention time and net charge of a HSP can be determined to determine the cell-penetrance and non-specific cell lysis activity. In certain embodiments, the HPLC retention time and calculated hydrophobicity of a HSP can be determined to determine the cell-penetrance and non-specific cell lysis activity. In certain embodiments, the calculated hydrophobicity and percent α-helicity of a HSP can be determined to determine the cell-penetrance and non-specific cell lysis activity. In certain embodiments, the percent α-helicity and net charge of a HSP can be determined to determine the cell-penetrance and non-specific cell lysis activity. In certain embodiments, the calculated hydrophobicity and net charge of a HSP can be determined to determine the cell-penetrance and non-specific cell lysis activity. In certain embodiments, the HPLC retention time, pI, and net charge of a HSP can be determined to determine the cell-penetrance and non-specific cell lysis activity.

In some embodiments, the HPLC retention time, calculated hydrophobicity, and net charge of a HSP can be determined to determine the cell-penetrance and non-specific cell lysis activity. In some embodiments, the isoelectric point (pI), HPLC retention time, and percent α-helicity of a HSP can be determined to determine the cell-penetrance and non-specific cell lysis activity. In some embodiments, the isoelectric point (pI), HPLC retention time, and calculated hydrophobicity of a HSP can be determined to determine the cell-penetrance and non-specific cell lysis activity. In some embodiments, the isoelectric point (pI), percent α-helicity, and net charge of a HSP can be determined to determine the cell-penetrance and non-specific cell lysis activity. In some embodiments, the isoelectric point (pI), percent α-helicity, and calculated hydrophobicity of a HSP can be determined to determine the cell-penetrance and non-specific cell lysis activity. In some embodiments, the percent α-helicity, HPLC retention and calculated hydrophobicity of a HSP can be determined to determine the cell-penetrance and non-specific cell lysis activity. In some embodiments, the percent α-helicity, HPLC retention and net charge of a HSP can be determined to determine the cell-penetrance and non-specific cell lysis activity.

In certain embodiments, the HPLC retention time, percent α-helicity, isoelectric point (pI), and net charge of a HSP can be determined to determine the cell-penetrance and non-specific cell lysis activity. In certain embodiments, the HPLC retention time, percent α-helicity, isoelectric point (pI), and calculated hydrophobicity of a HSP can be determined to determine the cell-penetrance and non-specific cell lysis activity.

In some embodiments, the at least one biophysical property of the HSP is HPLC retention time, percent α-helicity, isoelectric point (pI), calculated hydrophobicity, and net charge.

Methods are known in the art to determine plasma membrane lysis (or cell integrity), e.g., release of lactate dehydrogenase (LDH) (Decker and Lohmann et al., J. Immunol. Methods 1988; 115: 61-9 (see, Example 1), live/dead fluorescence assays (e.g., propidium iodide staining, or calcein/ethidium staining (Papadopoulos et al., J. Immunol. Methods 1994; 177(1-2): 101-111; and Wang et al., Hum. Immunol. 1993; 37(4): 264-270), and cytoplasmic swelling.

### Pharmaceutical Compositions

One or more (e.g., one, two, three, four, five, six, seven, eight, nine, ten, eleven, twelve, thirteen, fourteen, fifteen, sixteen, seventeen, eighteen, nineteen, or twenty) of the peptides (e.g., hydrocarbon-stapled and/or stitched peptides; exemplary stapled peptides include SEQ ID Nos: 2-38 and 40-49) disclosed herein can be formulated for use as or in pharmaceutical compositions. Such compositions can be formulated or adapted for administration to a subject via any route, e.g., any route approved by the Food and Drug Administration (FDA). Exemplary methods are described in the FDA Data Standards Manual (DSM) (available at http://www.fda.gov/Drugs/DevelopmentApprovalProcess/FormsSubmissionRequirements/ElectronicSubmissions/DataStandardsManualmonographs).

The pharmaceutical compositions of this invention may be administered, e.g., orally, parenterally, by inhalation spray or nebulizer, topically, rectally, nasally, buccally, vaginally, via an implanted reservoir, by injection (e.g., intravenously, intra-arterially, subdermally, intraperitoneally, intramuscularly, and/or subcutaneously), in an ophthalmic preparation, or via transmucosal administration. Suitable dosages may range from about 0.001 to about 100 mg/kg of body weight, or according to the requirements of the particular drug. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intra-arterial, intrasynovial, intrastemal, intrathecal, intralesional and intracranial injection or infusion techniques. Alternatively, or in addition, the present invention may be administered according to any of the methods as described in the FDA DSM.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound or agent disclosed herein which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

In some instances, pharmaceutical compositions can include an effective amount of one or more (e.g., one, two, three, four, five, six, seven, eight, nine, ten, eleven, twelve, thirteen, fourteen, fifteen, sixteen, seventeen, eighteen, nineteen, or twenty) stabilized peptides. The terms “effective amount” and “effective to treat,” as used herein, refer to an amount or a concentration of one or more (e.g., one, two, three, four, five, six, seven, eight, nine, ten, eleven, twelve, thirteen, fourteen, fifteen, sixteen, seventeen, eighteen, nineteen, or twenty) compounds or a pharmaceutical composition described herein utilized for a period of time (including acute or chronic administration and periodic or continuous administration) that is effective within the context of its administration for causing an intended effect or physiological outcome (e.g., treatment of infection).

The methods herein contemplate administration of an effective amount of compound or compound composition to achieve the desired or stated effect. Typically, the pharmaceutical compositions of this invention can be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

In some embodiments, an effective dose of a hydrocarbon-stapled and/or stitched peptide can include, but is not limited to, e.g., about, 0.00001, 0.0001, 0.001, 0.01, 0.1, 1 or 10-10000; 0.00001, 0.0001, 0.001, 0.01, 0.1, 1 or 10-5000; 0.00001, 0.0001, 0.001, 0.01, 0.1, 1 or 10-2500; 0.00001, 0.0001, 0.001, 0.01, 0.1, 1 or 10-1000; 0.00001, 0.0001, 0.001, 0.01, 0.1, 1 or 10-900; 0.00001, 0.0001, 0.001, 0.01, 0.1, 1 or 10-800; 0.00001, 0.0001, 0.001, 0.01, 0.1, 1 or 10-700; 0.00001, 0.0001, 0.001, 0.01, 0.1, 1 or 10-600; 0.00001, 0.0001, 0.001, 0.01, 0.1, 1 or 10-500; 0.00001, 0.0001, 0.001, 0.01, 0.1, 1 or 10-400; 0.00001, 0.0001, 0.001, 0.01, 0.1, 1 or 10-300; 0.00001, 0.0001, 0.001, 0.01, 0.1, 1 or 10-200; 0.00001, 0.0001, 0.001, 0.01, 0.1, 1 or 10-100; 0.00001, 0.0001, 0.001, 0.01, 0.1, 1 or 10-90; 0.00001, 0.0001, 0.001, 0.01, 0.1, 1 or 10-80; 0.00001, 0.0001, 0.001, 0.01, 0.1, 1 or 10-70; 0.00001, 0.0001, 0.001, 0.01, 0.1, 1 or 10-60; 0.00001, 0.0001, 0.001, 0.01, 0.1, 1 or 10-50; 0.00001, 0.0001, 0.001, 0.01, 0.1, 1 or 10-40; 0.00001, 0.0001, 0.001, 0.01, 0.1, 1 or 10-30; 0.00001, 0.0001, 0.001, 0.01, 0.1, 1 or 10-20; 0.00001, 0.0001, 0.001, 0.01, 0.1, 1 or 10-30; 0.00001, 0.0001, 0.001, 0.01, 0.1, 1-15, 0.00001, 0.0001, 0.001, 0.01, 0.1, 1 or 10-30; 0.00001, 0.0001, 0.001, 0.01, 0.1, 1-10, 0.00001, 0.0001, 0.001, 0.01, 0.1, 1 or 10-30; or 0.00001, 0.0001, 0.001, 0.01, 0.1, 1-5 mg/kg/day.

The compounds of this invention may be modified by appending appropriate functionalities to enhance selective biological properties (including, e.g., hydrophobicity and/or the position/occurrence of hydrophobic patches). Such modifications are known in the art and include those which increase biological penetration into a given biological compartment (e.g., blood, lymphatic system, central nervous system), increase oral availability, increase solubility to allow administration by injection, alter metabolism and alter rate of excretion.

An antimicrobial peptide selective for microbial versus mammalian membranes (i.e., a peptide able to kill or inhibit the growth of a microbe while also having a relatively low ability to lyse or inhibit the growth of a mammalian cell) may, e.g., possess a MIC for one or more (e.g., 1, 2, 3, 4, 5, 6, 7, 8, 9, 10, 11, 12, 13, 14, 15, 16, 17, 18, 19, or 20) microbes more than 1.5-fold lower, more than 2-fold lower, more than 2.5-fold lower, more than 3-fold lower, more than 4-fold lower, more than 5-fold lower, more than 6-fold lower, more than 7-fold lower, more than 8-fold lower, more than 9-fold lower, more than 10-fold lower, more than 15-fold lower, or more than 20-fold lower than the MIC of the corresponding parent (i.e., unmodified) non-internally cross-linked peptide for the same one or more (e.g., 1, 2, 3, 4, 5, 6, 7, 8, 9, 10, 11, 12, 13, 14, 15, 16, 17, 18, 19, or 20) microbes. In addition, an antimicrobial peptide selective for microbial versus mammalian membranes may lyse, e.g., less than 50%, less than 40%, less than 30%, less than 25%, less than 20%, less than 15%, less than 10%, less than 5%, less than 2.5%, less than 2%, or less than 1% of red blood cells (RBCs) in a RBC hemolytic activity assay when administered at its MIC for one or more (e.g., 1, 2, 3, 4, 5, 6, 7, 8, 9, 10, 11, 12, 13, 14, 15, 16, 17, 18, 19, or 20) microbes. The RBC hemolytic activity of an antimicrobial peptide selective for microbial versus mammalian membranes may be less than, approximately equal to, less than 1.5-fold greater, less than 2-fold greater, less than 2.5-fold greater, less than 3-fold greater, less than 4-fold greater, less than 5-fold greater, less than 6-fold greater, less than 7-fold greater, less than 8-fold greater, less than 9-fold greater, or less than 10-fold greater than the RBC hemolytic activity of the corresponding parent (i.e., unmodified) non-internally cross-linked peptide.

Hydrophobic patches within a peptide or protein may be identified using techniques generally known in the art, including, e.g., computational prediction/simulation (e.g., using ExPASy ProtScale, available at http://web.expasy.org/protscale/, Scooby-domain prediction, available at http://www.ibi.vu.nl/programs/scoobywww/, PSIPRED, available at http://bioinf.cs.ucl.ac.uk/psipred/, hydrophobic cluster analysis, see, e.g., http://www.impmc.upmc.fr/-callebau/HCA.html, Kyte Doolittle plotting, available at, e.g., http://gcat.davidson. edu/DGPB/kd/kyte-doolittle.htm or http://www.vivo.colostate.edu/molkit/hydropathy/index.html, and/or SPLIT, available at http://split.pmfst.hr/split/4/) and/or experimental determination (e.g., using techniques involving NMR spectroscopy, electron microscopy, homology modeling, small-angle X-ray and/or neutron scattering (SAXS/SANS), and/or X-ray crystallography) of the structure of the peptide or protein.

Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate, trifluoromethylsulfonate, and undecanoate. Salts derived from appropriate bases include alkali metal (e.g., sodium), alkaline earth metal (e.g., magnesium), ammonium, and N-(alkyl)4+salts. This invention also envisions the quaternization of any basic nitrogen-containing groups of the compounds disclosed herein. Water or oil-soluble or dispersible products may be obtained by such quaternization.

Pharmaceutical compositions of this invention can include one or more peptides and any pharmaceutically acceptable carrier and/or vehicle. In some instances, pharmaceuticals can further include one or more (e.g., 1, 2, 3, 4, 5, 6, 7, 8, 9, 10, 11, 12, 13, 14, 15, 16, 17, 18, 19, or 20) additional therapeutic agents in amounts effective for achieving a modulation of disease or disease symptoms.

When the compositions of this invention comprise a combination of a compound of the formulae described herein and one or more (e.g., 1, 2, 3, 4, 5, 6, 7, 8, 9, 10, 11, 12, 13, 14, 15, 16, 17, 18, 19, or 20) additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, e.g., ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, may also be advantageously used to enhance delivery of compounds of the formulae described herein.

The pharmaceutical compositions can contain any conventional non-toxic pharmaceutically-acceptable carriers, adjuvants or vehicles. In some cases, the pH of the formulation can be adjusted with pharmaceutically acceptable acids, bases or buffers to enhance the stability of the formulated compound or its delivery form. The term parenteral as used herein includes subcutaneous, intra-cutaneous, intra-venous, intra-muscular, intra-articular, intra-arterial, intra-synovial, intra-sternal, intra-thecal, intra-lesional and intra-cranial injection or infusion techniques.

### EXAMPLES

### Example 1: Materials and Methods

### Stapled Peptide Synthesis and Characterization

All hydrocarbon-stapled peptides were synthesized, derivatized at the N-terminus with FITC-βAla or acetyl, and purified to >95% homogeneity by LC/MS using methods known in the art and as previously described³⁴. Acetylated peptides were dissolved in 10% (vol/vol) acetonitrile in water for circular dichroism analyses, performed on an Aviv Biomedical spectrophotometer using methods known in the art and as previously described³⁴.

### ImageXpress Microscopy Analysis

For high-content fluorescence microscopy analysis, the indicated cell lines were plated in black, clear bottom plates overnight at a density of 2×10⁴ cells per well in DMEM supplemented with 10% (vol/vol) FBS, 1% penicillin/streptomycin, and 1% glutamine. The following day, cells were treated with 0.5 μM FITC-labeled peptides or the equivalent amount of vehicle (0.1% DMSO) for 4 h in serum-free DMEM, and then stained with Hoechst 33342 and CellMask Deep Red (CMDR, Invitrogen) for 10 min. The media was aspirated, and cells were fixed with 4% (wt/vol) paraformaldehyde for 10 min, washed three times with PBS and imaged by ImageXpress Microscopy (high-throughput epifluorescence microscope; Molecular Devices). Data were collected for four sites per well at 20× magnification, with each treatment performed in duplicate, and then analyzed and quantified using MetaXpress software. The CMDR stain was used to visualize the boundaries of the cell and to create a mask for measuring FITC-peptide inside the cell, thereby excluding fluorescent debris from the analysis. A custom module in MetaXpress was applied to incrementally recede the CMDR image mask from the cellular border, further restricting the analyzed FITC signal to internalized peptide. The analysis module was calibrated by defining uniformly negative vs. uniformly positive total well fluorescence based on a vehicle-treated field and a validated positive control field, respectively (e.g., BIM SAHB_{A1}^{10,20}); the orthogonal measure of TIFI then determined the level of absolute fluorescence detected per cell, per peptide construct, for the intra-panel comparisons. Maximum and minimum thresholding was utilized to exclude FITC and Cy5 outliers that were much larger and brighter than average and total intensity, and average intensity per cell thresholds were set such that nearly all vehicle-treated cells scored negative by the analysis. For each comparative analysis, all stapled peptides in the panel were measured on the same day using the same plating of cells and peptide dilutions. Then, the entire experiment was repeated twice more (three biological replicates overall) on different days, with freshly plated cells and peptide dilutions.

### Cell Culture

B-ALL (BCL-X_{L}-reconstituted p185⁺Arf^{-/-}Mcl-1^{del})^{25,26} and Jurkat T cells (ATCC, TIB-152) were maintained in RPMI 1640 (ATCC) supplemented with 10% (v/v) FBS, 100 U/mL penicillin, 100 mg/mL streptomycin, 0.1 mM MEM nonessential amino acids, and 50 mM 3-mercaptoethanol. Mouse embryonic fibroblast (MEF) and HeLa cells were maintained in DMEM high glucose (Invitrogen) supplemented with 10% (v/v) FBS, 100 U/mLI penicillin, 100 mg/mLI streptomycin, 2 mM L-glutamine, 50 mM HEPES, 0.1 mM MEM nonessential amino acids, and 50 mM 3-mercaptoethanol. Cells were verified to be mycoplasma-free using the MycoAlert™ mycoplasma detection kit (Lonza Biologics).

### Lactase Dehydrogenase Release Assay

MEFs were plated in 96-well format (1.5×10⁴ cells per well), and after overnight incubation, full media was replaced with serum-free DMEM. B-ALL and Jurkat T cells were plated in 96-well format (2×10⁴ cells per well) in serum-free RPMI. Serial dilutions of BIM-SAHB_{A1} from a 10 mM DMSO stock, or vehicle, were added to the MEFs in a final volume of 100 μL and incubated at 37° C. for 0.5 or 3 h, or the peptides were added to the B-ALL or Jurkat T cells at 10 μM and incubated for 0.5 h. The plate was centrifuged at 1500 rpm for 5 min at 4° C., and 80 μL of cell culture media was transferred to a clear plate (Corning), incubated with 80 μL of LDH reagent (Roche) for 15 min while shaking, and absorbance measured at 490 nm on a microplate reader (SpectraMax M5 Microplate Reader, Molecular Devices).

### Recombinant Protein Production

BCL-X_{L} ΔC was expressed as a glutathione-S-transferase (GST) fusion protein in Escherichia coli BL21 (DE3) from the pGEX2T vector (Pharmacia Biotech) and purified by affinity chromatography using glutathione sepharose beads (GE Healthcare), followed by thrombin cleavage of the GST tag and gel filtration FPLC, performed using methods known in the art and as previously described²⁵.

### Fluorescence Polarization Binding Assay

For direct fluorescence polarization (FP) binding assays, F1TC-derivatized peptides (25 nM) were added to serial dilutions of recombinant protein in binding buffer (100 mM NaCl, 50 mM Tris, pH 8.0) in 96-well black opaque plates. The plates were incubated in the dark at room temperature and then fluorescence polarization was measured at 20 min on a microplate reader (SpectraMx M5 Microplate Reader, Molecular Devices). IC₅₀s were calculated by nonlinear regression analysis of dose-response curves using Prism software 5.0 (GraphPad).

### Cell Viability

B-ALL cells (4×10⁴/well) were seeded in 96-well opaque plates in a volume of 40 μL in serum-free RPMI using a multiwell dispenser (Apricot) to ensure consistency and reproducibility among the 5-7 plates required per panel. A 2× concentrated plate of the indicated serial dilutions of peptide (10 mM DMSO stock) or DMSO (0.4%) in serum-free RPMI in a volume of 40 μL was then transferred to the cells and the plate incubated at 37° C. for 4 hours, at which time, 8.9 μL per well of 100% FBS was added back and cell viability assayed 20 h later by addition of CellTiter-Glo reagent, according to the manufacturer's protocol (Promega).

### Statistical Methods

The Spearman's correlation coefficient was used to assess the degree of the univariate relationships between the TIFI and the biophysical variables in the dataset. This quantity is based on the ranks of the data rather than on the observed data values, and consequently is less sensitive to outliers or extreme values than Pearson. The significance of the Spearman's correlation coefficient is evaluated by means of a permutation test, with the calculations performed using the R statistical software³⁵ package pvrank³⁶. Data are represented graphically by scatterplots, and lines were fit using a loess smoother.

Principal components analysis (PCA)²¹⁻²³ provides a useful method to observe the data through a coordinate system that highlights variability. Retaining an interpretable number of principal components, each of which is a weighted combination of a subset of the available variables, reduces the dimensionality of the data by simplifying the interpretation of the covariance structure. The first principal component explains the largest proportion of the variance, and so on. The original covariates for the exploratory PCA included TIFI, net charge, pI, hydrophilicity, percent hydrophobic residues, hydrophobicity, hydrophobic moment (vector components of magnitude and direction), pH 7 retention time, and percent α-helicity. If only the first three principal components are retained, this procedure can be used to describe the data within a three-dimensional space. Using the first three principal components, we were able to capture 93.5% and 96.2% of the variability of the staple walk and point mutation datasets, respectively. The calculations were performed using Stata v.13.1³⁷.

Further analysis of the data from a different standpoint was conducted by using a recursive partitioning algorithm³⁸. The result is a tree-like representation, calculated using the R package rpart²⁴, and includes “relevant” binary splits of the dataset: the optimal cut points for each split are evaluated by assessing sums of squares, as in analysis of variance.

### Example 2: Development and Validation of a High-Throughput/High-Stringency Microscopy Assay for Stapled Peptide Internalization

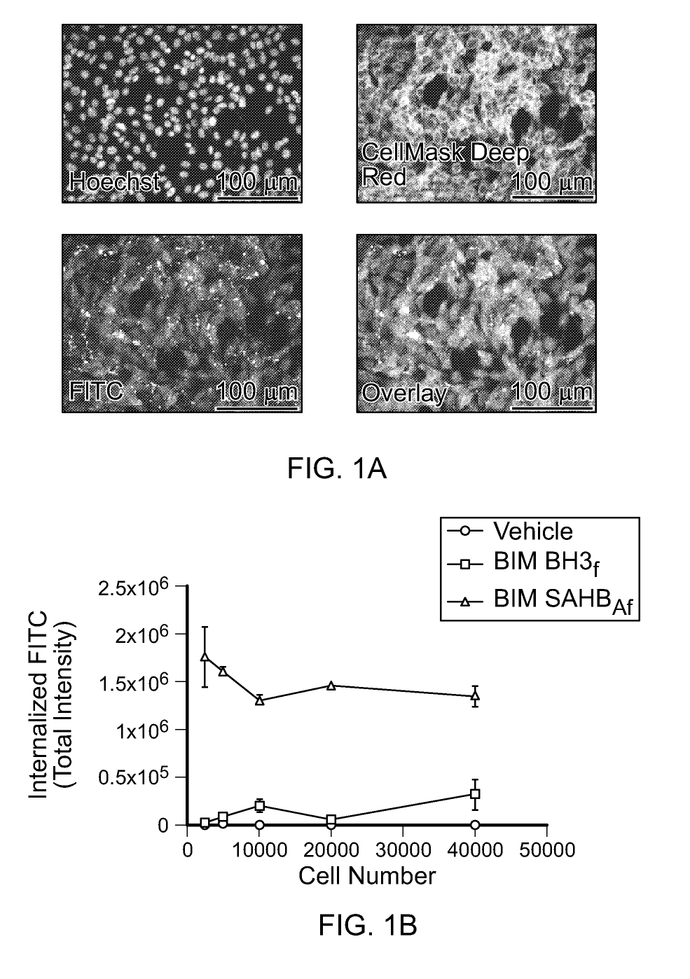

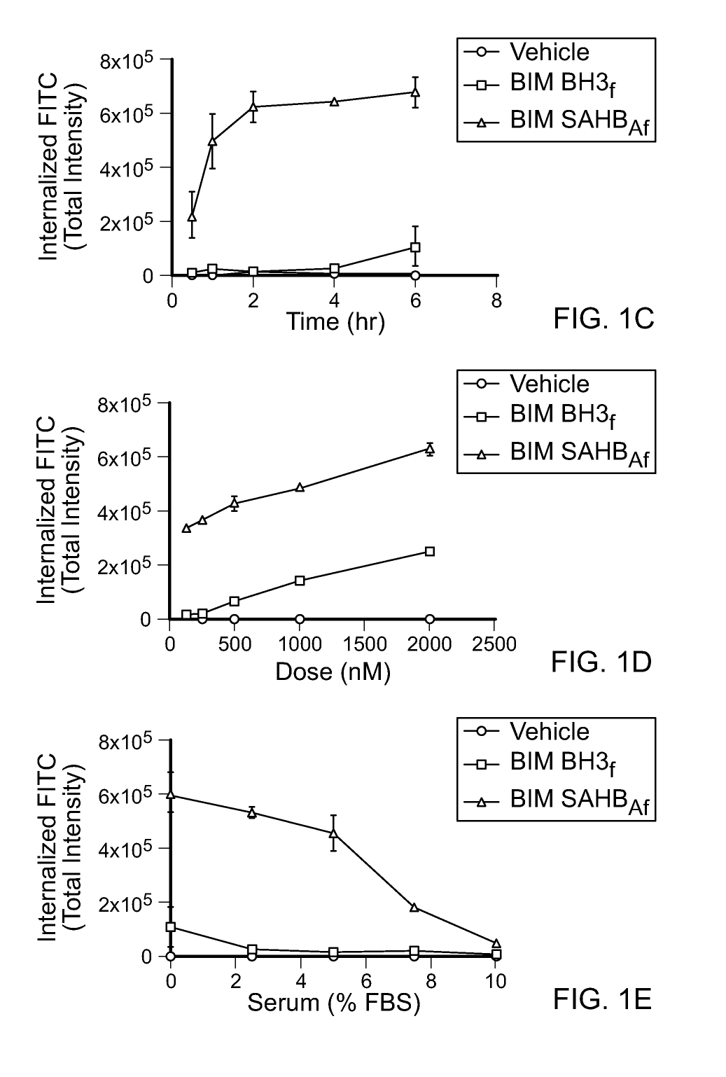

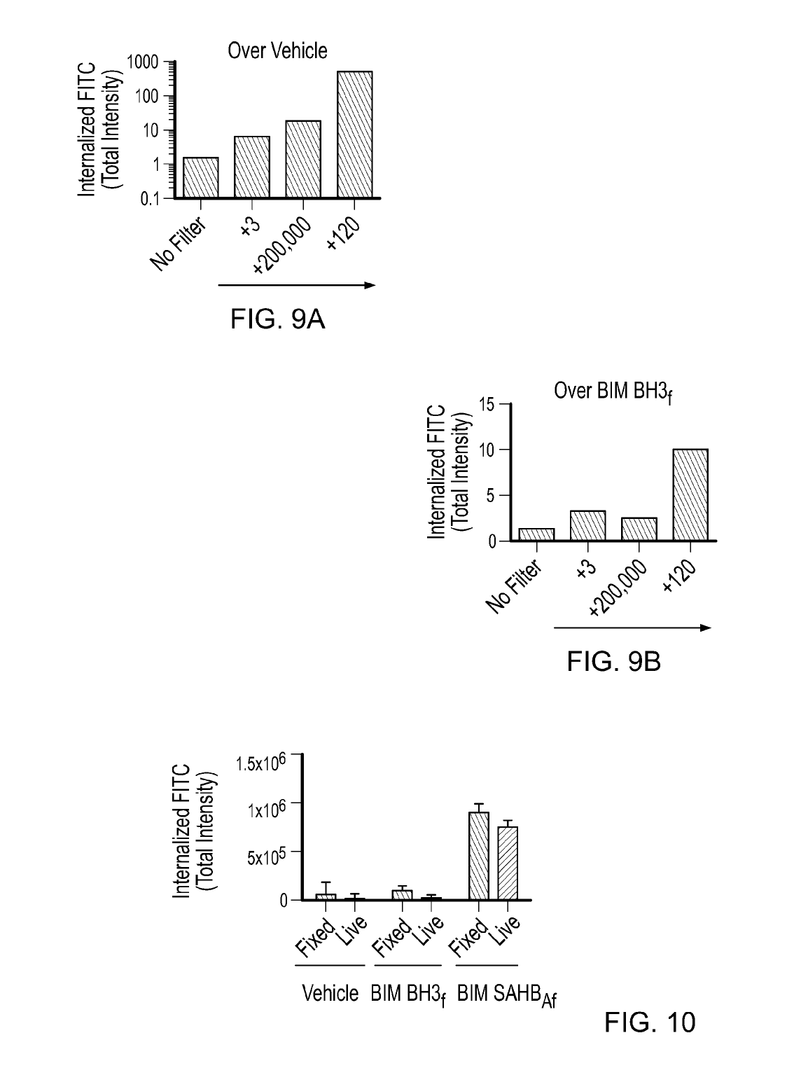

High-content epifluorescence microscopy and the ImageXpress Micro (IXM) Widefield High Content Analysis System were used to develop a rigorous quantitation platform for measuring cellular uptake of stapled peptides derivatized at the N-terminus with a FITC fluorophore. Custom Module Editor was used to create a custom module (CM) for analysis based on the following principles: (1) define cellular uptake in mouse embryonic fibroblasts (MEFs) based on visualizing cellular and nuclear boundaries using Hoechst 33342 and CellMask Deep Red (CMDR), respectively, thereby excluding extracellular aggregates and autofluorescent debris (FIG. 1A), (2) contract the resultant cellular mask by a defined pixel boundary to avoid quantitation of extracellular, membrane-adherent peptide, (3) exclude FITC signal outliers that reflect out-of-focus fluorescence, (4) acquire images for analysis at 20× and 40× magnification to ensure precise resolution of fluorescence on a per cell basis, (5) visualize four distinct central locations of the cellular field to achieve unbiased sampling and avoid edge artifact (FIG. 8), and (6) maximize signal-to-noise ratios to ensure optimal sensitivity and specificity of internal FITC-peptide signal (FIGS. 9A-B). Using this CM, it was confirmed that fixation with 4% paraformaldehyde had no effect on the total internalized FITC intensity (TIFI) measurements when compared to live cell imaging, enabling us to eliminate any variability that could arise from subjecting live cells to acquisition times lasting up to 2 hours (FIG. 10).

Next optimal cellular density, acquisition time point, and peptide dose for measuring internalized FITC-peptide were determined. Comparing vehicle, the unmodified template peptide FITC-BIM BH3₁ (aa 146-166) (SEQ ID NO: 1) and the corresponding stapled analog BIM SAHB_{A1} (SEQ ID NO: 10) at 500 nM dosing for 4 hours, robust TIFI was detected only for BIM SAHB_{A1}, with the signal essentially constant on a per cell basis across the 2.5×10³ to 4.0×10⁴ range of cellular densities tested (FIG. 1B). Subsequently TIFI was followed over time upon treating MEFs at a density of 2×10⁴ cells with 500 nM peptides, and observed time-responsive uptake for BIM SAHB_{A1} that peaked by 2 hours and remained stable thereafter (FIG. 1C). Using a plating density of 2×10⁴ and time point of 2 hours, the dose of applied peptide was varied and a linear, dose-responsive increase in TIFI for FITC-BIM BH3₁ and FITC-BIM SAHB_{A1} was observed with the stapled analog consistently showing markedly enhanced uptake over the entire dose range (FIG. 1D). To avoid any differences in peptide internalization based on variability in serum binding, the above analyses were conducted in serum-free medium. However, given the importance of understanding the influence of serum exposure on the cellular activity of stapled peptides, the effect of fetal bovine serum (FBS) on peptide internalization over a 0% to 10% dose range was tested. Consistent with prior observations that FBS can suppress the cellular uptake and biological activity of BIM SAHB_{A1}^{8,10}, the serum-suppressive effect was dose-responsive, with near complete elimination of cellular uptake at 10% FBS but relative preservation of cellular uptake up to 5% of added FBS (FIG. 1E).

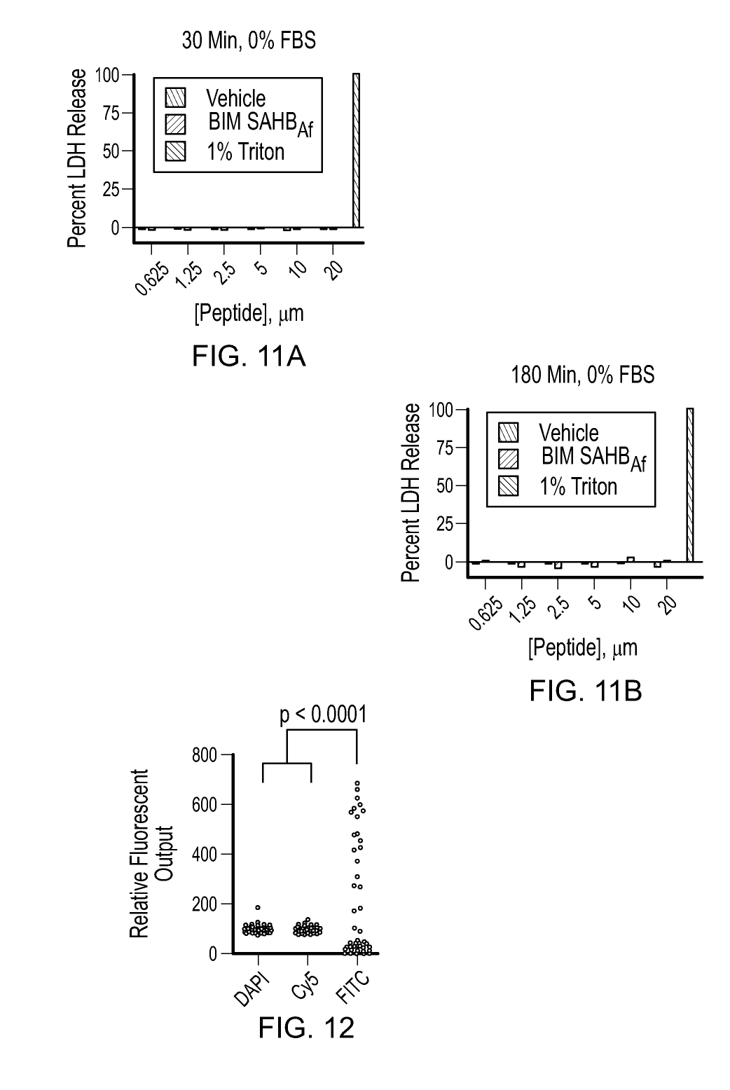

To explore the hypothesis that stapled peptides may gain cellular entry by inducing plasma membrane lysis and that FBS suppresses this uptake by preserving membrane integrity¹⁹, MEFs at a plating density of 2×10⁴ were subjected to a serial dilution of FITC-BIM SAHB_{A1} starting at 20 μM in the absence of serum and monitored cellular lysis by LDH release over time. Importantly, no LDH release was observed across the entire dose range at either 30 min or 180 min post-treatment (FIG. 11). These data rule out the possibility that FITC-BIM SAHB_{A1} achieves cellular entry via membrane lysis or that FBS is blocking stapled peptide import by preventing membrane lysis. Thus, based on the above results, (1) 2×10⁴ cell density, (2) 4-hour acquisition time, (3) 500 nM dosing, and (4) 0% FBS were selected as treatment parameters in order to maximize sensitivity and specificity of the present detection method while avoiding the variability introduced by live cell imaging and differential effects of serum on the diverse compositions within the stapled peptide libraries.

### Example 3: Determinants of Cellular Uptake for a Staple Scanning Library of BIM BH3

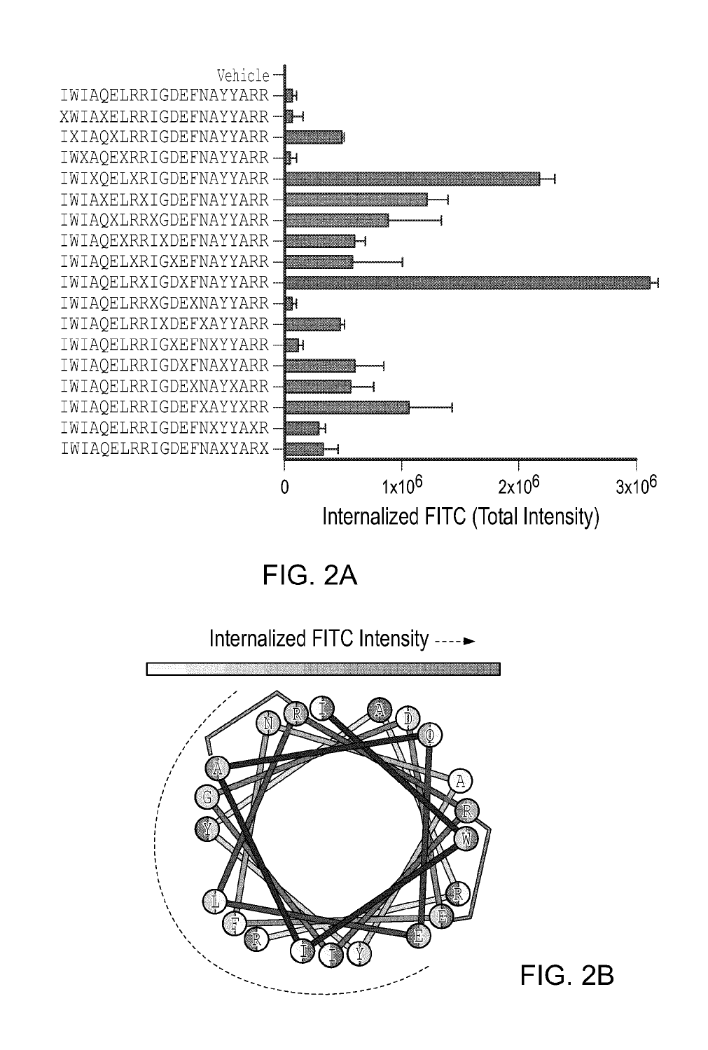

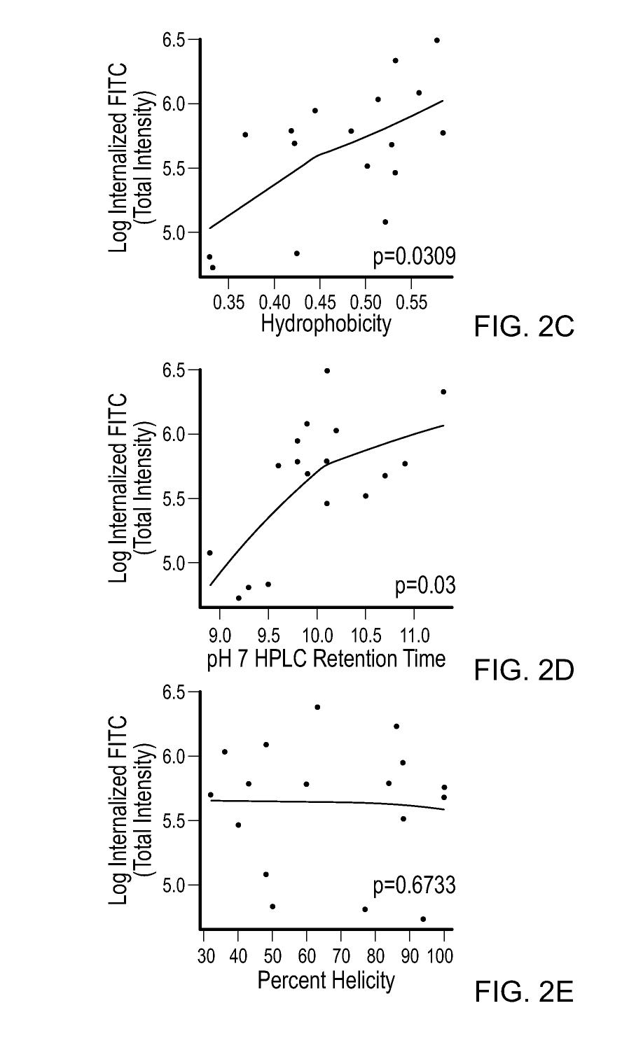

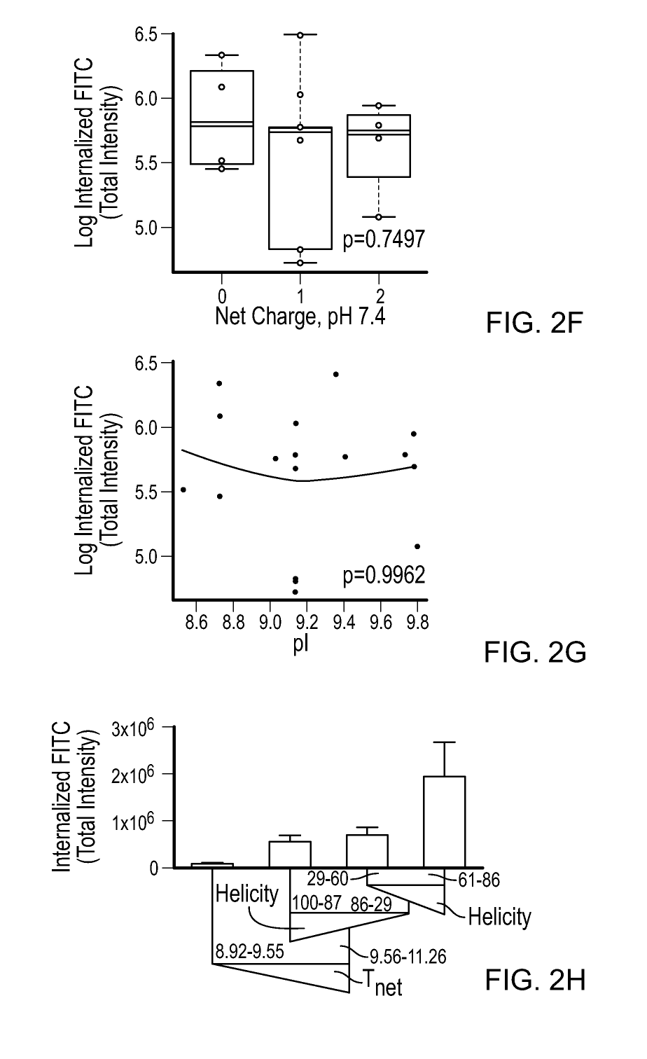

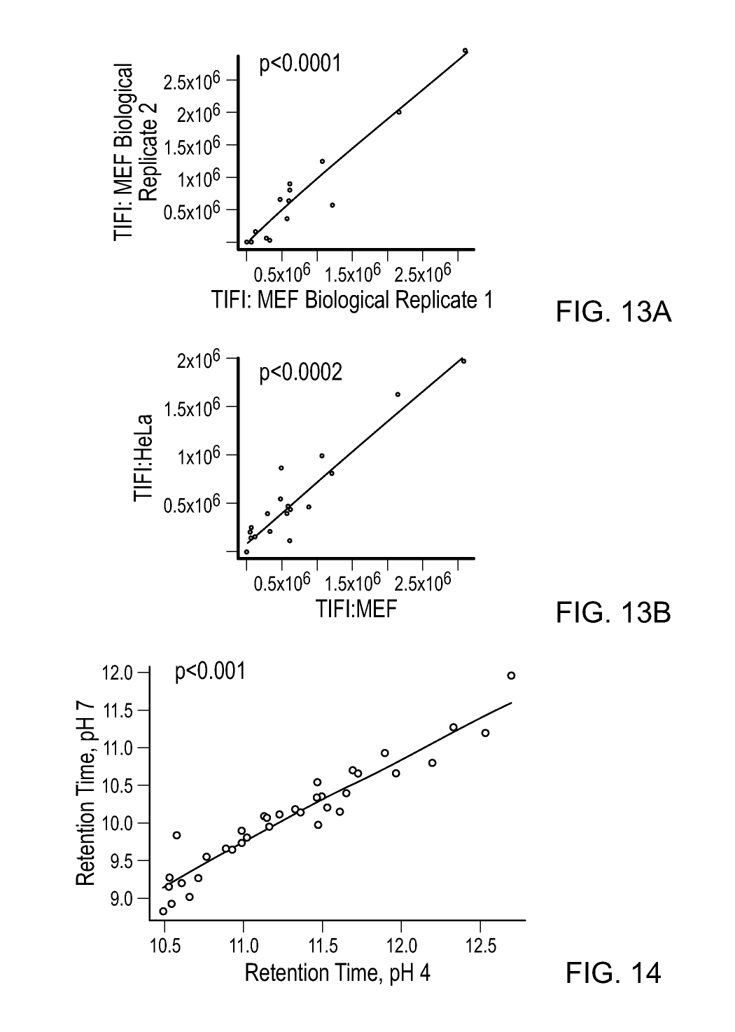

When designing stapled peptides for biochemical and biological studies, one of the first questions to address is where to install the staple. Here, a library of stapled BIM BH3 peptides was generated by performing a “staple scan” that sequentially places the staple along the length of the template peptide, yielding 17 i, i+4 stapled peptides. Cellular uptake of the FITC-derivatized stapled peptides was monitored by IXM using the previously described CM. Importantly, of the cellular fluorescent dyes measured in the microscopy assay (i.e., Hoechst, CMDR, FITC), only the FITC intensity varied with stapled peptide composition (FIG. 12). Strikingly, our prototype BIM SAHB_{A1} peptide that bears a staple flanking IGD emerged as the clear winner (TIFI>3.0×10⁶), with four additional constructs containing staples flanking QEL, ELR, AYY, and LRR also demonstrating notable uptake at TIFIs of 2.16×10⁶, 1.21×10⁶, 1.07×10⁶, and 0.88×10⁶, respectively (FIG. 2A). The reproducibility of both the microscopy method and TIFI values for comparative uptake analyses were verified by performing biological replicates, which showed strong mutual association (Spearman's correlation p<0.0001; FIG. 13A). To examine whether the TIFI hierarchy observed in MEFs was recapitulated in a distinct cell line, the analyses were repeated in HeLa cells and again observed strong association (Spearman's correlation p=0.0002; FIG. 13B), reinforcing the technical and biological reproducibility of the presently described quantitative approach.

In examining the staple distribution of the most penetrant constructs along the amphipathic helix using an intensity scale, staple positions located at the hydrophobic/hydrophilic boundary were the most favorable for cellular uptake (FIG. 2B). These data suggest that extending the hydrophobic surface of stapled peptides beyond 180 degrees, as in the case of BIM BH3, may be a critical design feature of cell-penetrant stapled peptides. Indeed, all six BIM SAHB constructs bearing a staple restricted to the hydrophobic face of the helix were among the least cell-permeable peptides.

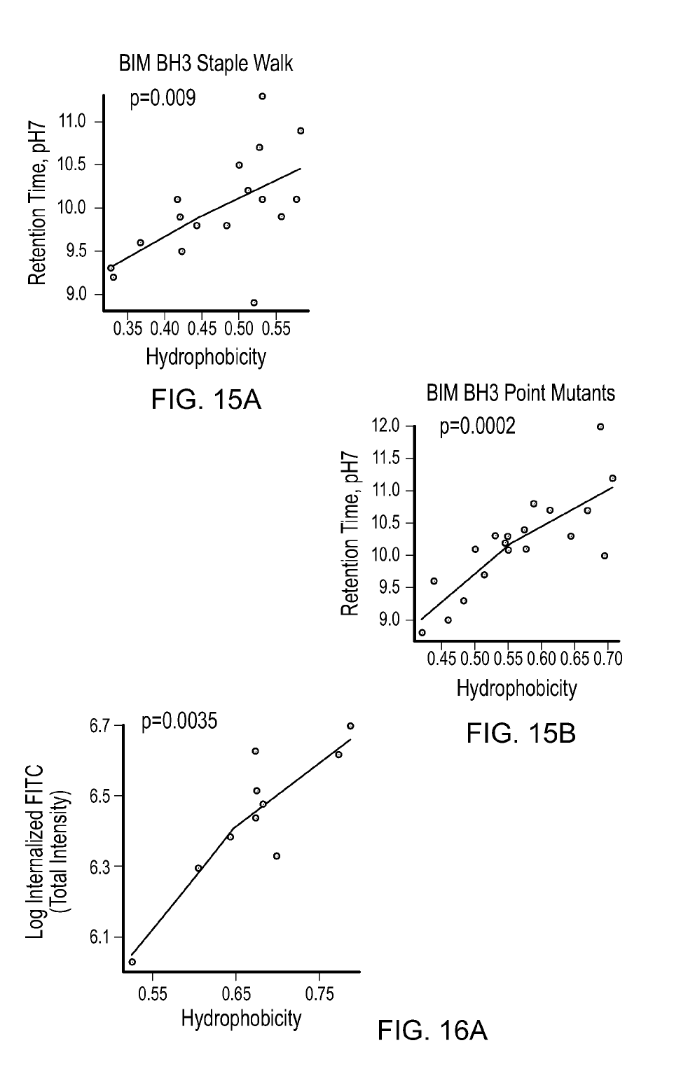

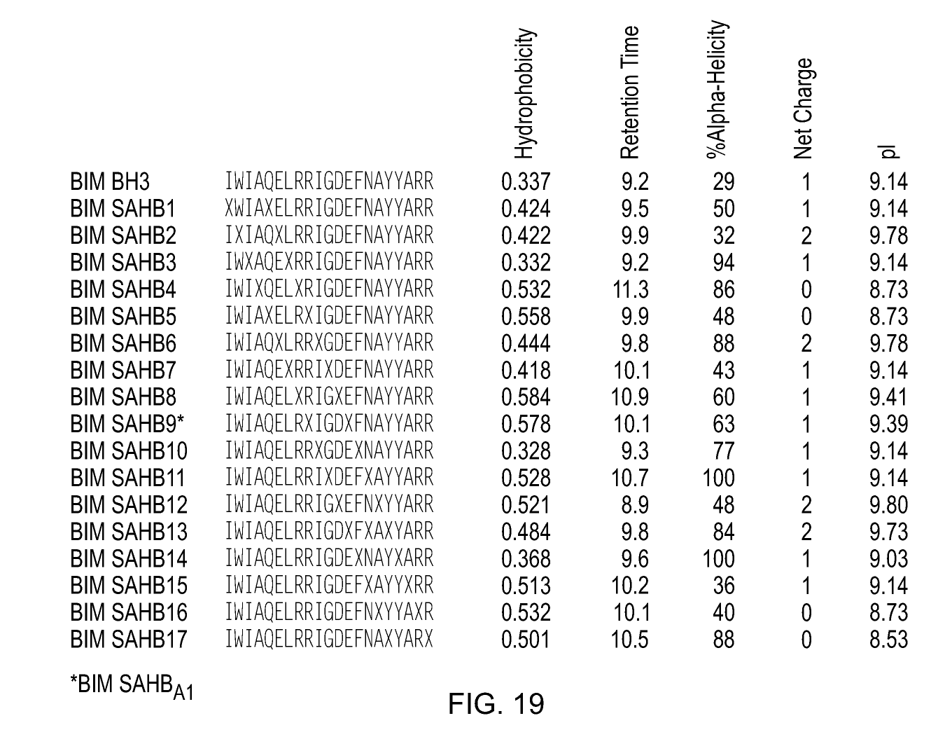

To expand this type of know-how beyond empiric observation, a table of calculated and experimental parameters was compiled that defined key biophysical properties of the stapled peptide library, including calculated hydrophobicity, HPLC column retention time (pH 7), percent α-helicity (circular dichroism), and calculated net charge and pI (FIG. 19). Only the calculated hydrophobicity and experimentally-determined HPLC retention time (pH 7) demonstrated independent, near-linear, and statistically-significant predictive value of cellular uptake (p-value for Spearman's correlation test of 0.031 and 0.030, respectively), with the association following a piecewise linear trajectory (FIGS. 2C-G). Of note, HPLC retention times were determined at pH 7 to gauge peptide behavior at physiologic pH, but also found that the relative retention times at pH 4—the more typical pH for HPLC purifying peptides—correlated exquisitely well with the pH 7 values (p-value for Spearman's correlation test<0.001), thereby allowing retention time analyses for these purposes to be conducted under routine conditions (FIG. 14). Because calculated hydrophobicity and HPLC retention time also correlated extremely well with one another (p-value for Spearman's correlation test of 0.009) (FIG. 15), our data suggest that staple placement at the amphipathic boundary combined with prioritization of constructs based on relatively high hydrophobicity as determined from calculations alone, reflect a critical first step in designing a cell-penetrant stapled peptide.

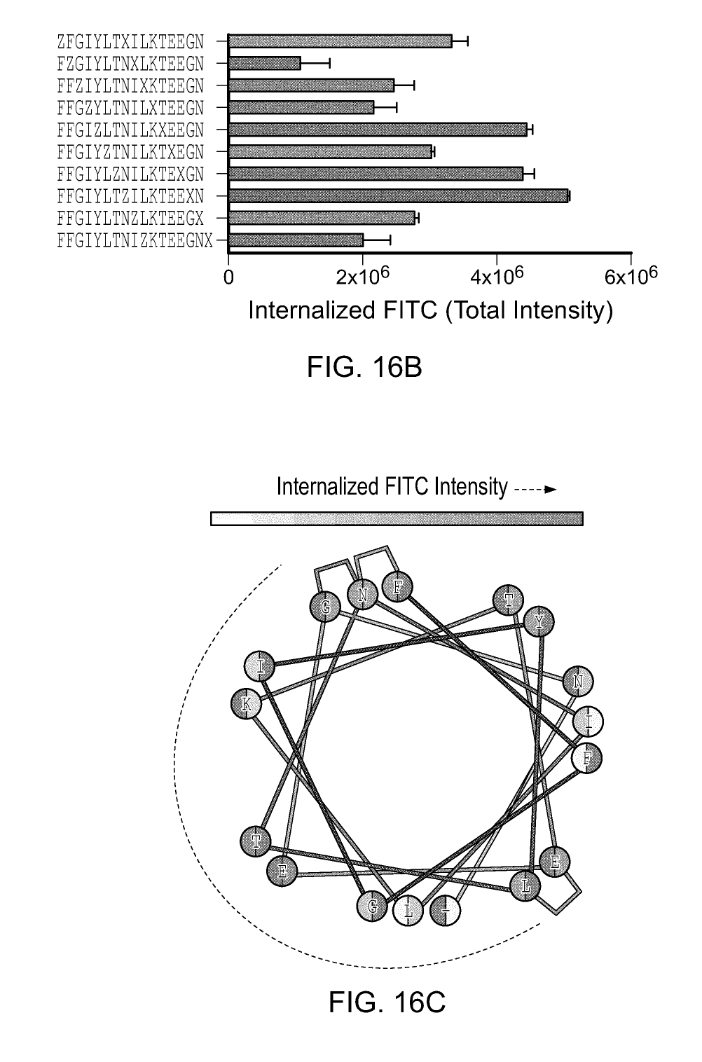

Indeed, these design conclusions were verified using a different sequence template bearing less amphipathic character and an alternative i, i+7 staple composition, namely stabilized alpha-helices of SOS1 (SAH-SOS1) (SEQ ID NO: 39). Again, cellular uptake, as reflected by TIFI measurement, is highly associated with hydrophobicity (Spearman's correlation p=0.035) (FIG. 16A) and all three of the staples that extend the hydrophobic surface beyond the target protein binding interface are among the constructs yielding the highest TIFI values (FIG. 16B).

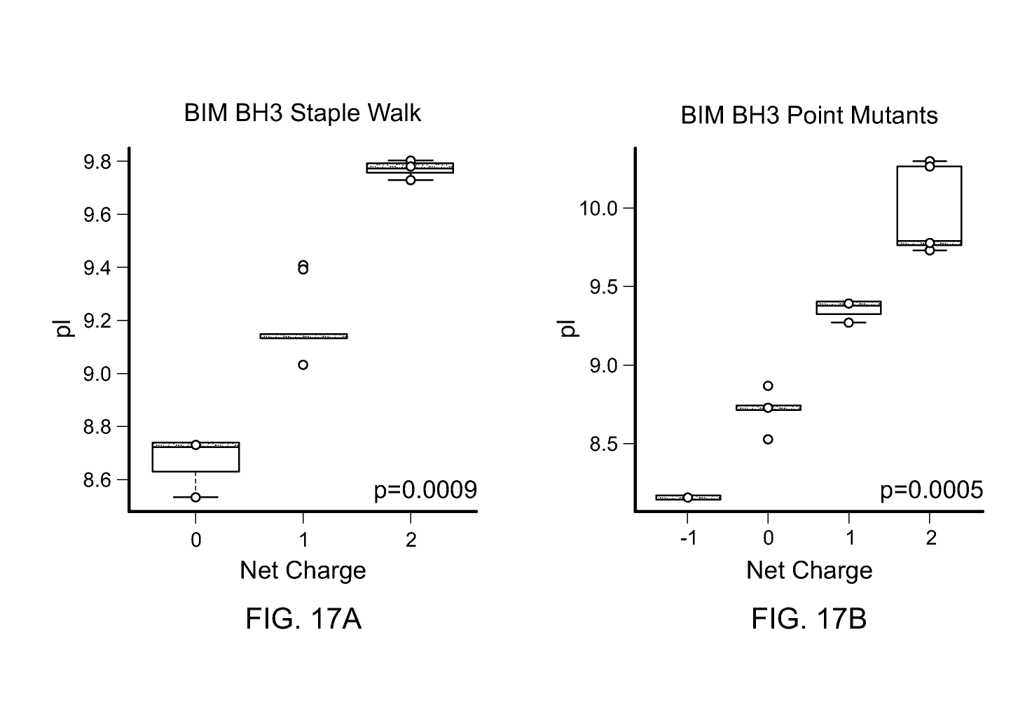

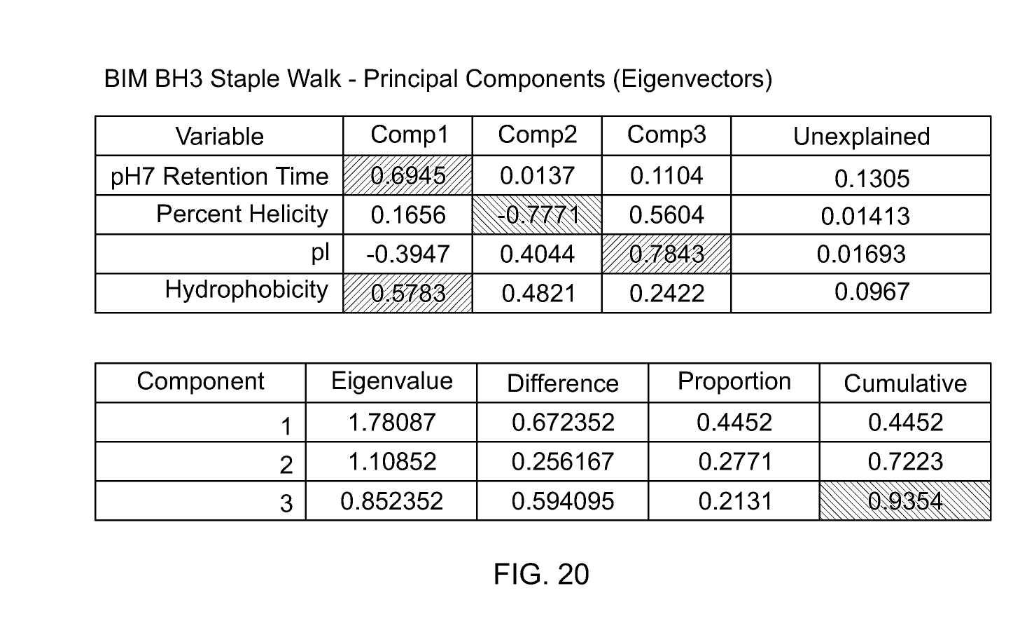

To determine if biophysical features beyond hydrophobicity influenced cellular uptake in combinatorial fashion, the previously described table of parameters was subjected to principal component analysis (PCA)²¹⁻²³ that included 9 potential explanatory variables and TIFI, and the measures of hydrophobicity, pI/charge and α-helicity were identified as the primary features of the overall variability. Therefore, a subsequent PCA was performed using representative covariates for these quantities. Because overall charge and pI are both calculated values that correlate extremely well with one another (p-value for Spearman's correlation test<0.0001) (FIGS. 17A-B)), pI was selected to represent peptide charge state for this analysis. Both HPLC retention time and calculated hydrophobicity contributed strongly to principal component 1 (FIG. 14), whereas percent α-helicity and pI defined principal components 2 and 3, respectively (FIG. 20). Together, these parameters accounted for 93.5% of the observed variability in the cellular uptake data (FIG. 20). This result indicates that although stapled peptide hydrophobicity and retention time have a major (even primary) influence on stapled peptide uptake (representing 44% of the observed data variability by PCA), and although these two parameters are the only individual parameters to demonstrate a statistically-significant relationship with TIFI (FIGS. 2C-G), they do not account for the entirety of the observed variability in the stapled peptide uptake data. The PCA suggests that α-helical content and peptide charge serve as key secondary contributors to the data variability (28% and 21%, respectively).

To delve further into what degree of hydrophobicity, α-helical content and charge state together influenced the cellular uptake of our staple scanning library, the dataset was split using a recursive partitioning algorithm²⁴. As expected, the first breakpoint for distinguishing between cell permeable and impermeable peptides was based on HPLC retention time (FIG. 2H). All peptides with a retention time of less than 9.56 minutes uniformly demonstrated little to no cellular uptake. Of the stapled peptides that exhibited 9.56 minutes or greater retention time (i.e., the more hydrophobic subgroup), percent α-helicity then became a major driver. Interestingly, those stapled peptides with α-helicities of >87% or <60% demonstrated only moderate cellular uptake and notably less than constructs with α-helicities in the 61-86% range (FIG. 2H). Thus, the “sweet spot” for cellular uptake of stapled BIM peptides is dictated by relatively high hydrophobicity combined with elevated, but not excessive, α-helical content, an important subtlety masked by other modes of analysis.

### Example 4: Influence of Point Mutagenesis in Refining Cell-Penetrant Stapled Peptides

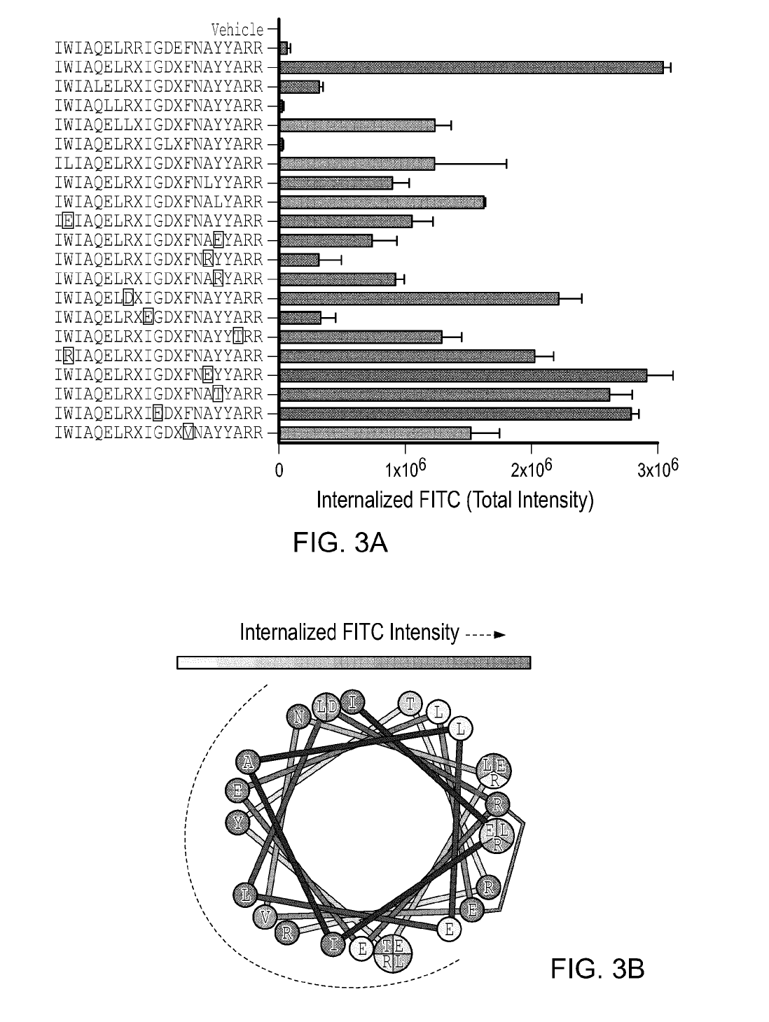

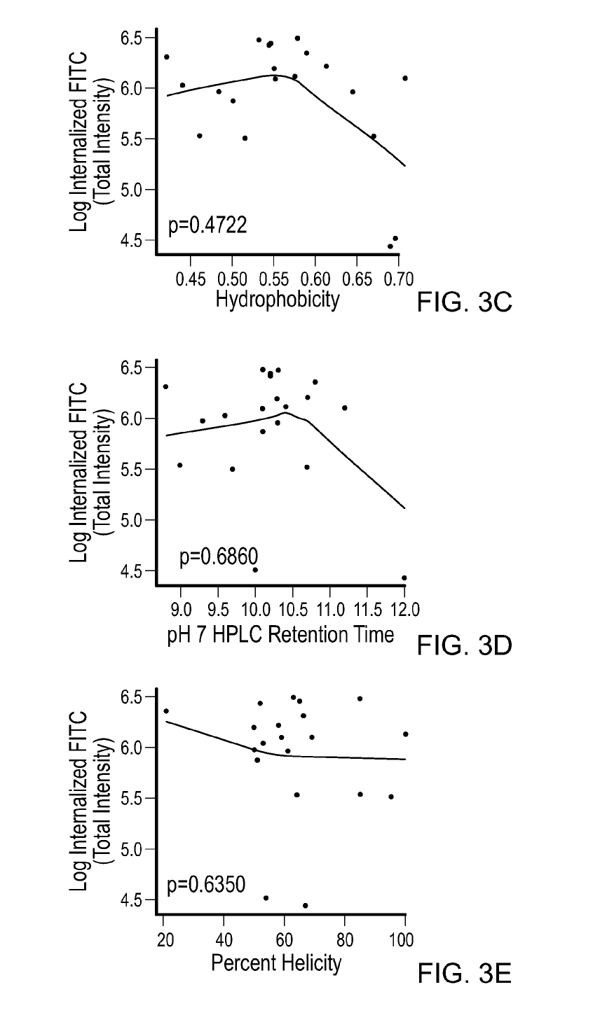

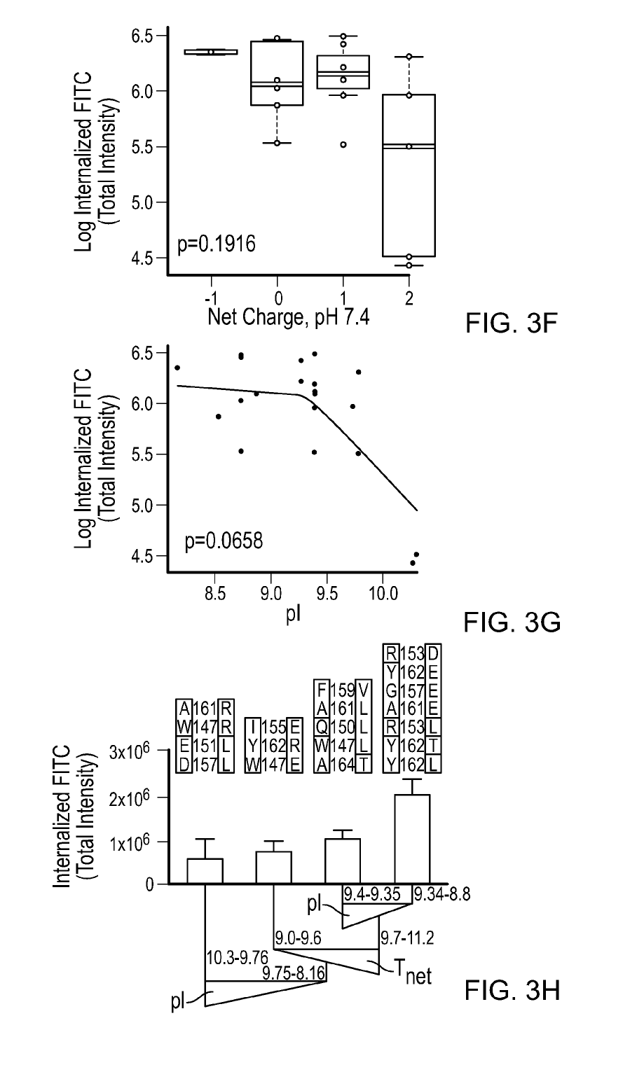

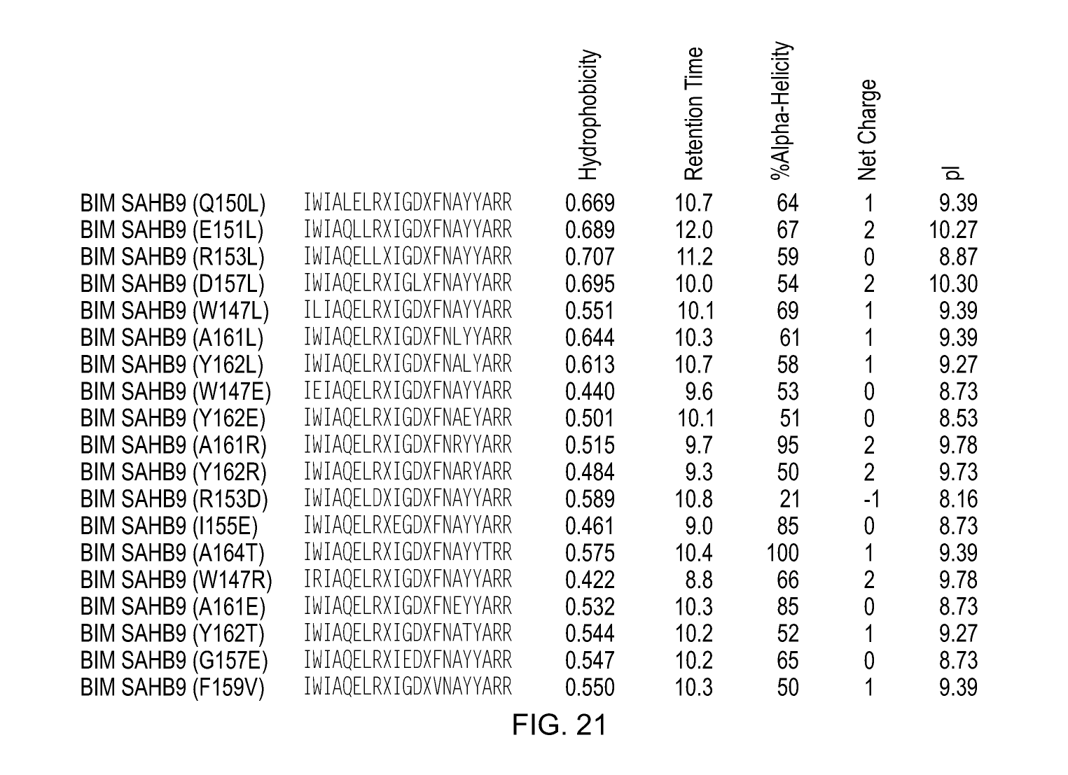

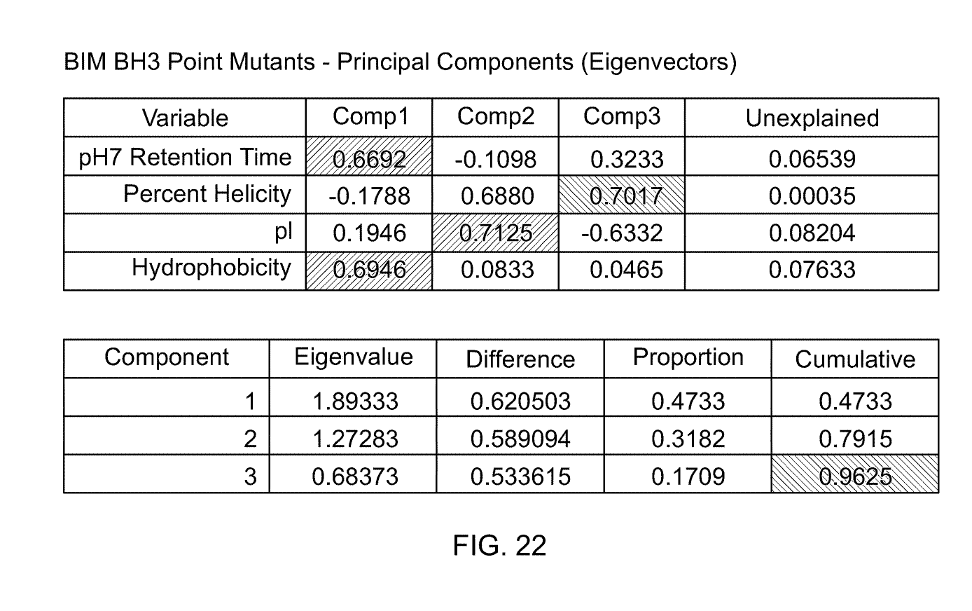

Once a lead stapled peptide emerges based on staple placement and the above-described hydrophobicity and α-helicity considerations, it may be desirable to generate an expanded cluster of leads by fine tuning amino acid composition, including evaluating the impact of adjusting overall charge—especially, e.g., in the context of particular stapled peptide applications^{3,4,9,11,12}. To better understand the influence of charge in the context of an already well-optimized stapled peptide lead, a BIM SAHB_{A1} library comprised of a series of point mutations that altered charge and hydrophobicity at discrete positions was generated (FIG. 3A, FIG. 21). Cellular uptake analysis of this library revealed a remarkable influence of single point mutations on TIFI (FIGS. 3A-B). Consistent with a combinatorial role of biophysical influences on stapled peptide uptake, no single parameter independently showed an association with TIFI for this point mutant library. Instead, PCA revealed that a cumulative 96% of the variability in the cellular uptake data could be explained by hydrophobicity/retention time (component 1, 47%), pI (component 2, 32%), and percent α-helicity (component 3, 17%) (FIG. 22). Thus, similar to the previous case, hydrophobicity/retention time explains the largest amount of variability. Interestingly, recursive partitioning demonstrated that mutations that either increased the pI (>9.75) or reduced the retention time (<9.7) relative to BIM SAHB_{A1} impaired cellular uptake, whereas constructs that maintained or increased retention time (9.7-11.2) and even lowered the overall pI (8.8-9.34) maintained high level cellular uptake (FIG. 3H). These data are inconsistent with the notion that positive charge is an independent, mechanism-based requirement for stapled peptide import¹⁴, as evidenced by retention of high level TIFI upon R153D, G156E, and A161E mutagenesis of BIM SAHB_{A1}. Taken together, cellular uptake of stapled BIM peptides is predominantly driven by hydrophobicity and α-helicity, yet because of multifactorial influences that include pI, expanding and fine tuning a pool of lead compounds can be achieved by judicious sequence modification.

### Example 5: Identifying Cell-Penetrant Stapled Peptides with Biological Activity/Function

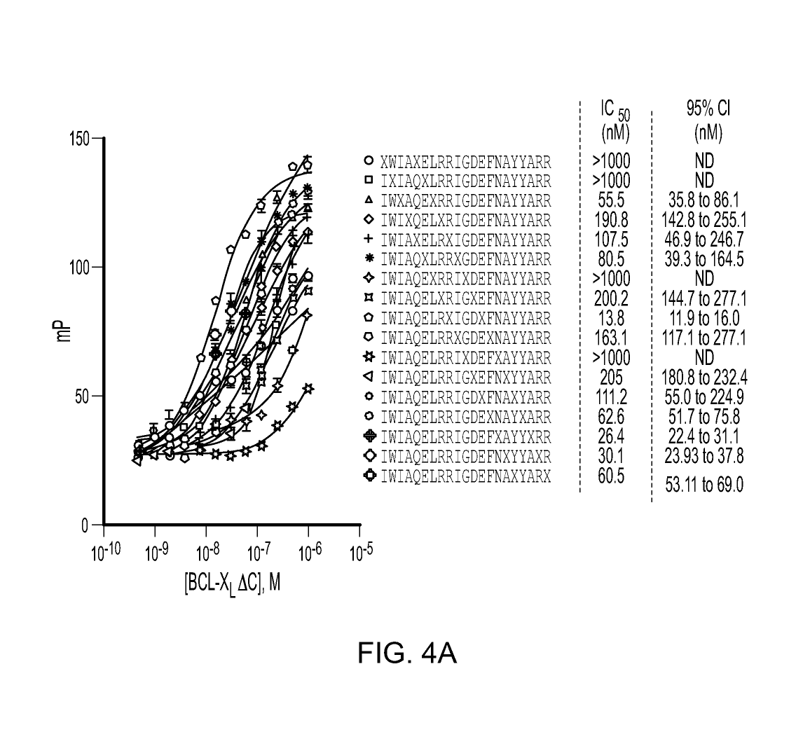

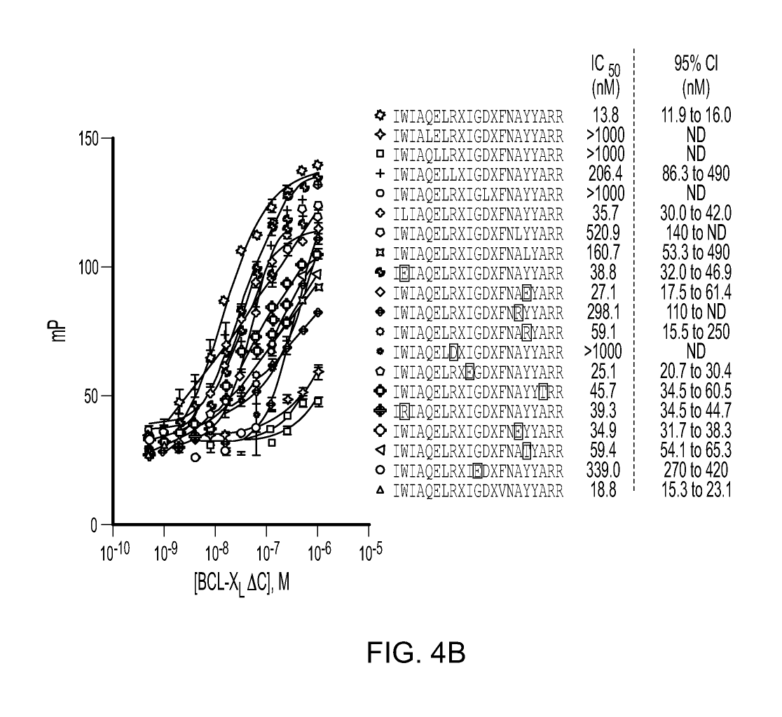

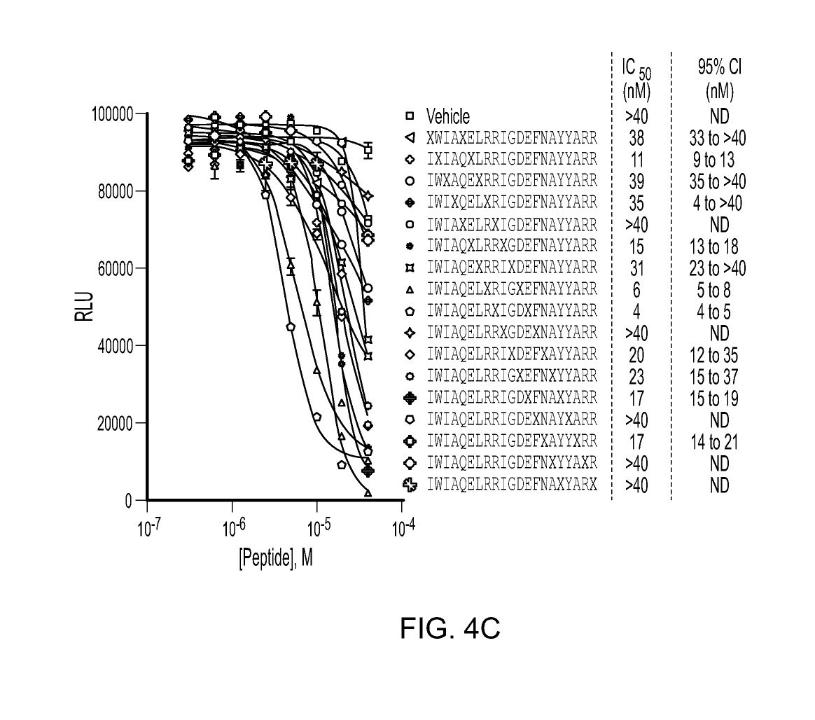

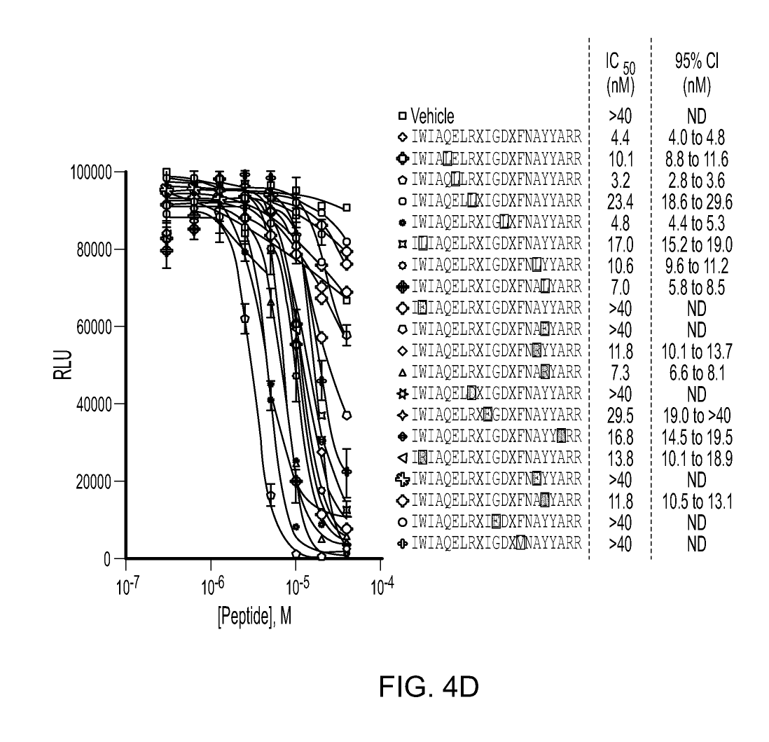

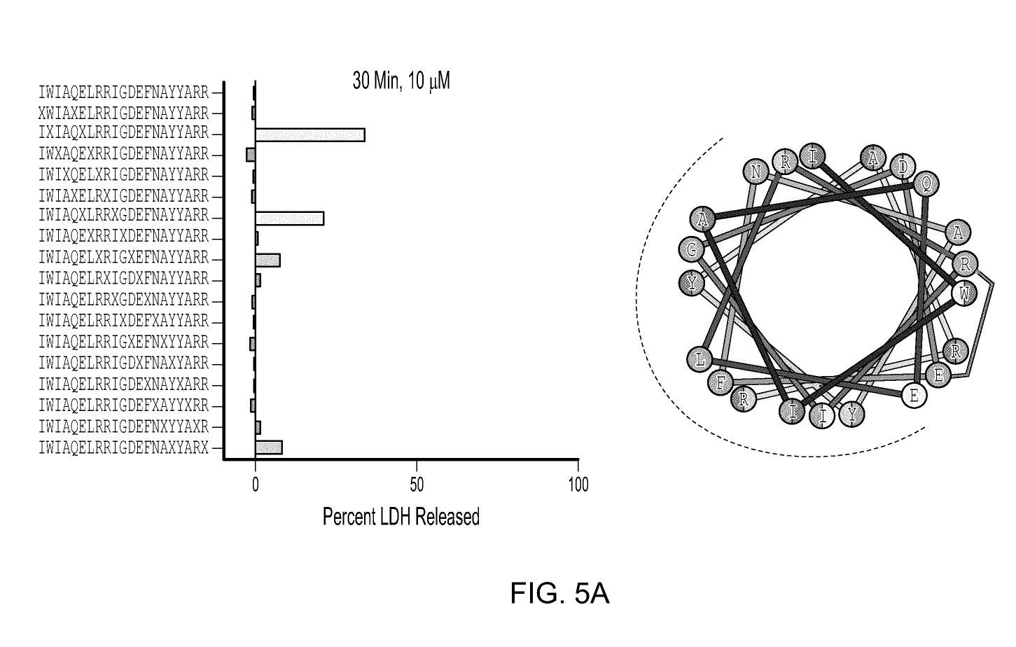

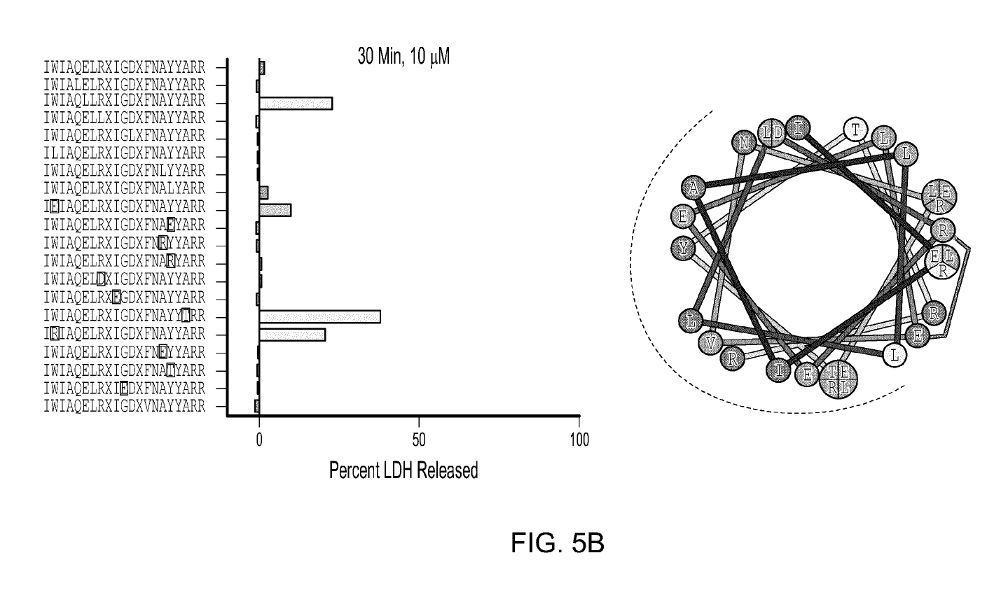

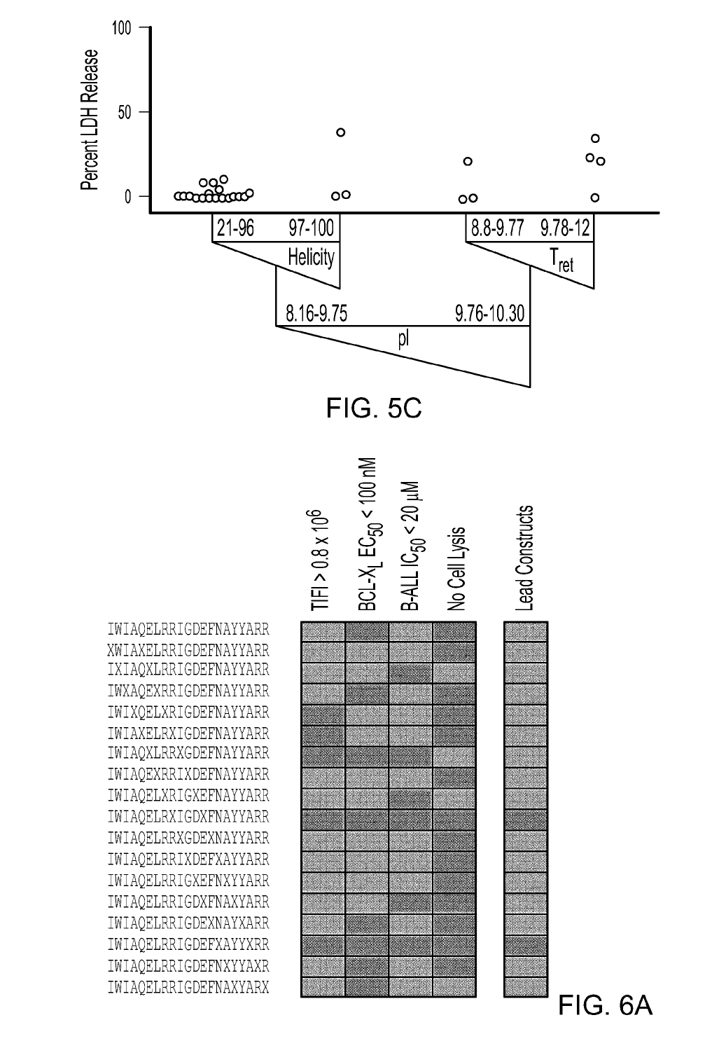

Because staple insertion and point mutagenesis by definition alter the native sequence of the template peptide, an essential step toward identifying cell-penetrant stapled peptides that are also bioactive involves biochemical and/or biological testing. For example, when the staple scanning and point mutation libraries of FITC-BIM BH3 were subjected to fluorescence polarization binding analysis against an established BCL-2 family target, such as BCL-X_{L}, a spectrum of binding affinities ranging from 14 nM (BIM SAHB_{A1}) to over 1 M were identified. The observed losses in binding activity are consistent with disrupting the critical hydrophobic interaction surface and/or mutating specific residues known to be essential for BCL-X_{L} interaction (e.g. L152, G156) (FIGS. 4A-B). Next the libraries were screened for cytotoxic activity in a leukemia cell line engineered to be dependent on BCL-X_{L} for survival (BCL-X_{L}-reconstituted p185⁺Arf^{-/-}Mcl-1-deleted B-cell acute lymphoblastic leukemia^{25,26}), and again obtained a spectrum of cytotoxic activity, with IC₅₀s ranging from 3 to >40 μM (FIGS. 4C-D). In order to identify compounds whose cytotoxicity correlates with the capacity for cellular uptake and intracellular target engagement, it is important to rule out nonspecific cytotoxicity due to potential plasma membrane lysis. This undesirable activity was screened by exposing cells to high micromolar dosing of stapled peptides, followed by measurement of LDH release after 30 min of treatment. 28 of 36 constructs (˜80%) showed no lytic activity when applied to the B-ALL cells at 10 μM dosing for 30 min, with 3 constructs demonstrating low grade lytic activity and 5 constructs identified as clearly lytic.

Of the 5 notably lytic stapled peptides, 3 had in common the conversion of E151 to a hydrophobic residue (i.e., stapling amino acid substitution or leucine mutation), with W147R and A164T mutagenesis also found to promote lysis. Thus, lytic properties can be attributed to very specific, focal changes in sequence composition, rather than reflecting a general liability associated with installing hydrocarbon staples.

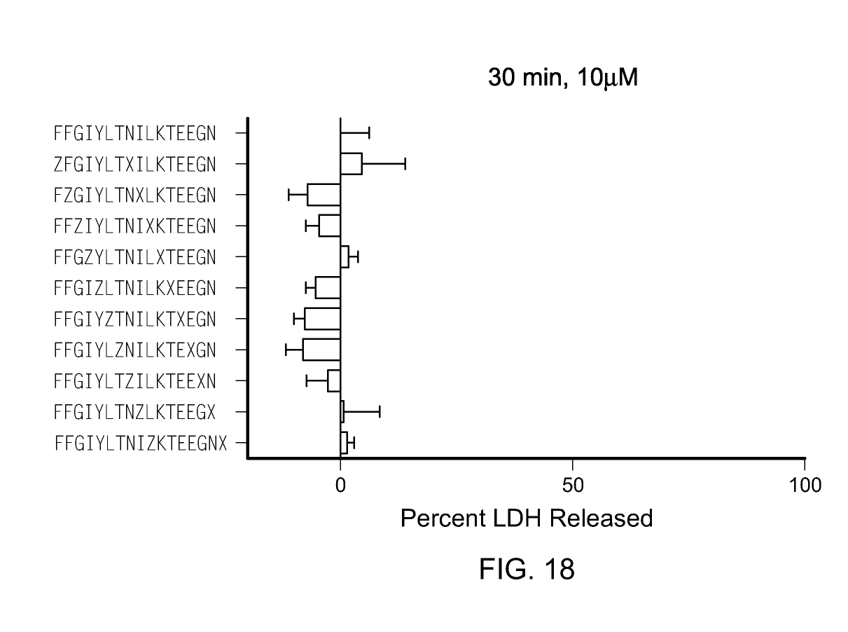

To better predict what biophysical parameters give rise to stapled peptides that are lytic, recursive partitioning analysis was applied using LDH release as the unwanted outcome. Interestingly, pI served as the first breakpoint for classifying stapled peptides as at risk for causing lysis or not. For the 26 of 36 constructs that displayed no lytic activity, the pI was <9.76 and α-helical content spanned a broad range from 21 to 96%. Only one peptide with pI<9.76 but α-helicity>97% caused membrane lysis, implying that near perfect α-helical stabilization could be detrimental. Of those stapled peptides with pIs in the 9.76-10.30 range, 1 of 3 peptides with retention times of 8.8-9.77 min and 3 of 4 peptides with retention times of 9.78-12 min were lytic. Thus, a combination of relatively high positive charge and hydrophobicity represent the greatest risk factors for generating stapled peptides with the propensity to lyse cellular membranes at high dosing levels. To test this conclusion, LDH release assays were performed using the distinct i, i+7-stapled SOS1 library and found that none of the SAH-SOS1 peptides induced plasma membrane lysis, consistent with all constructs having a pI of <9.76 (FIG. 18).

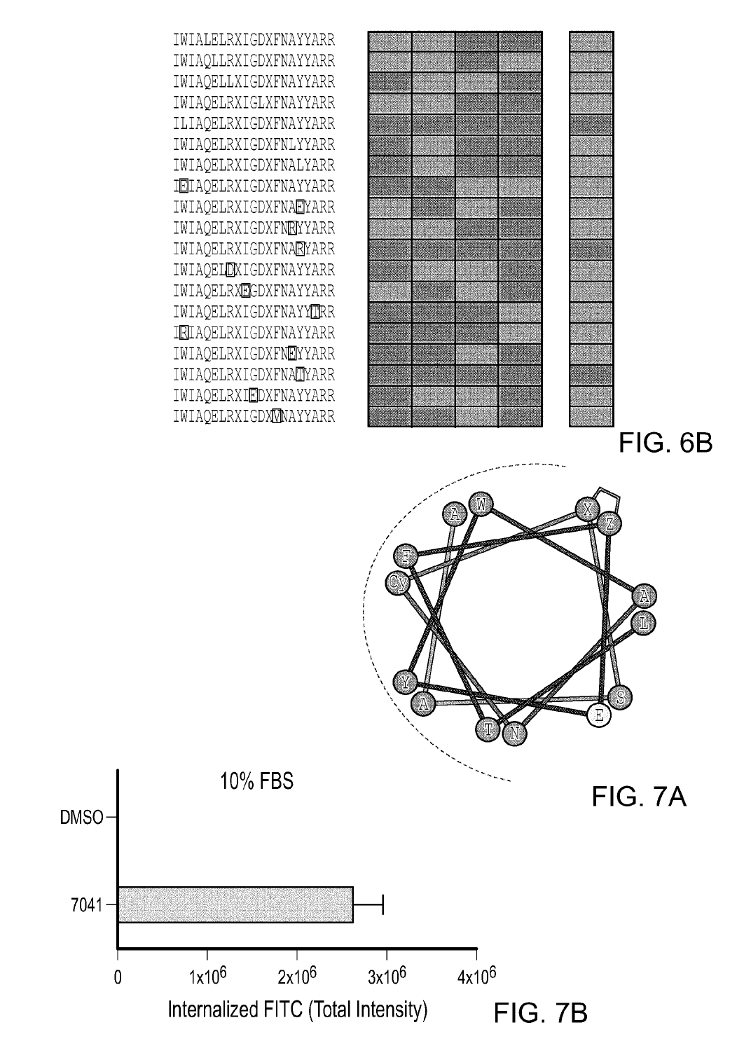

With cellular uptake, biochemical, and cellular data in hand, desired thresholds can then be applied to prioritize lead compounds for in vitro and in vivo application. For example, using selection criteria of TIFI>0.8×10⁶, BCL-X_{L} binding activity of <100 nM, and cellular IC₅₀<20 μM, BIM BH3 peptides bearing XIGDX- and XAYYX-positioned staples emerged as the most promising constructs for BCL-X_{L} targeting (FIG. 6). Indeed, the XIGDX staple (previously reported “A” position) has been the most successful to date when applied in the context of BID³, BIM^{8,10}, BAD, and PUMA²⁷ BH3 peptides, whereas installing a staple at the corresponding XAYYX position in MCL-1²⁸ BH3 yielded an optimal construct for structural and cellular work. Importantly, these analyses can also inform the selection of negative control point mutants that maintain efficient cellular uptake but lose binding and thus cellular activity as a result of disrupting essential amino acid contacts for protein target interaction, as exemplified by the R153D¹⁰ and G156E mutations in BIM BH3. Importantly, constructs that manifest cellular uptake, target protein binding affinity, and cytotoxicity but also induce membrane lysis can be identified and disqualified from further development (e.g., XLRRX staple in BIM BH3). Alternatively, compounds that are taken up by cells and bind the target but have weak cellular activity (e.g. XELRX position) or that exhibit relatively weak binding affinity but manifest cytotoxicity in the absence of significant membrane lysis, could warrant further affinity optimization or investigation of other cellular target(s), respectively.

Taken together, these data demonstrate a practical and unbiased approach for identifying a lead peptide such as BIM SAHB_{A1} (TIFI>3×10⁶, target protein binding EC₅₀ of 14 nM, cellular IC₅₀ of 4 μM, and no membrane lysis), for advancement as both an experimental reagent/tool and a potential therapeutic.

### Example 6: ATSP-7041

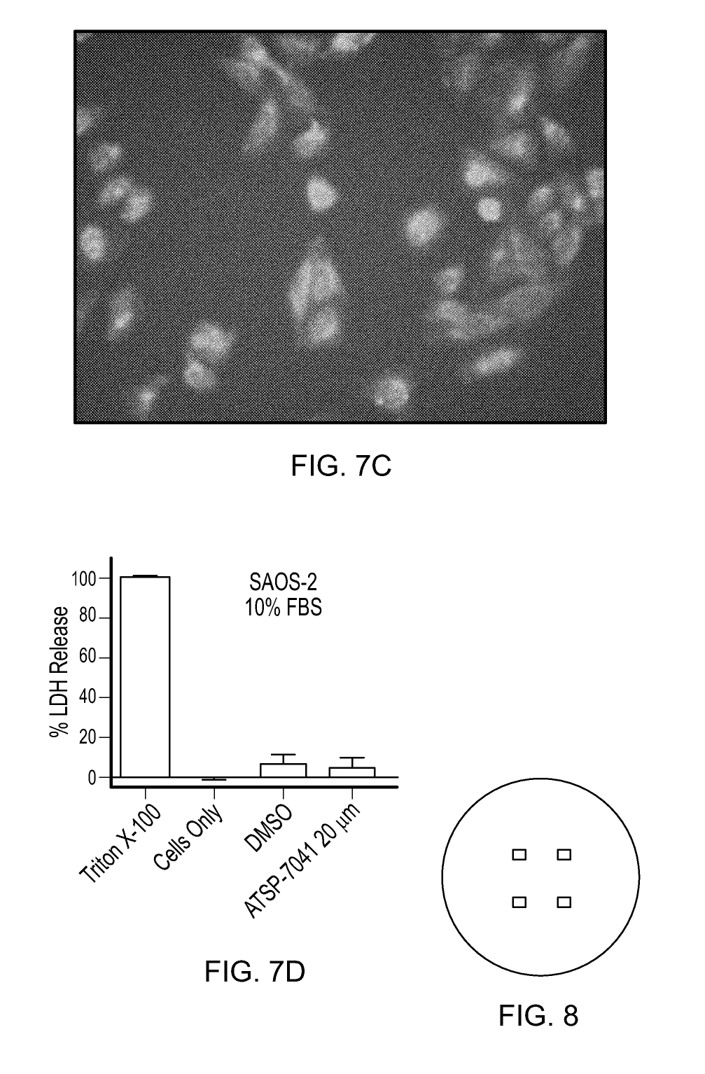

The stapled peptide ATSP-7041 (sequence LTFZEYWANCbXSAA; SEQ ID NO: 38, “X” represents S-pentenyl alanine; “Z” represents R-octenyl alanine; and “Cb” is cyclobutylalanine) was identified as possessing favorable hydrophobicity (0.84), retention time (10.4), α-helicity (70%), isoelectric point (7.1), and net charge (−1) parameters for cellular uptake. Further, ATSP-7041 contains an i, i+7 staple located at the boundary of the binding interface, effectively extending the hydrophobic surface, in accordance with the design principles described herein (FIG. 7A). Epifluorescence microscopy analysis confirmed that ATSP-7041 indeed demonstrates effective cellular penetrance (as evidenced by TIFI value) (FIGS. 7B-C). ATSP-7041 also lacks nonspecific membrane lytic properties, as evidenced by an LDH release assay (FIG. 7D).

### REFERENCES

1. Walensky, L. D. & Bird, G. H. Hydrocarbon-stapled peptides: principles, practice, and progress. J Med Chem 57, 6275-88 (2014).
2. Schafmeister, C., Po, J. & Verdine, G. An all-hydrocarbon cross-linking system for enhancing the helicity and metabolic stability of peptides. J Am Chem Soc 122, 5891-5892 (2000).
3. Walensky, L. D. et al. Activation of apoptosis in vivo by a hydrocarbon-stapled BH3 helix. Science 305, 1466-70 (2004).
4. Bemal, F., Tyler, A. F., Korsmeyer, S. J., Walensky, L. D. & Verdine, G. L.

Reactivation of the p53 tumor suppressor pathway by a stapled p53 peptide. J Am Chem Soc 129, 2456-7 (2007).
5. Bemal, F. et al. A stapled p53 helix overcomes HDMX-mediated suppression of p53.

Cancer Cell 18, 411-22 (2010).
6. Chang, Y. S. et al. Stapled alpha-helical peptide drug development: a potent dual inhibitor of MDM2 and MDMX for p53-dependent cancer therapy. Proc Natl Acad Sci USA 110, E3445-54 (2013).
7. Barclay, L. A. et al. Inhibition of Pro-apoptotic BAX by a noncanonical interaction mechanism. Mol Cell 57, 873-86 (2015).
8. Gavathiotis, E. et al. BAX activation is initiated at a novel interaction site. Nature 455, 1076-81 (2008).
9. Kim, W. et al. Targeted disruption of the EZH2-EED complex inhibits EZH2-dependent cancer. Nat Chem Biol 9, 643-50 (2013).
10. LaBelle, J. L. et al. A stapled BIM peptide overcomes apoptotic resistance in hematologic cancers. J Clin Invest 122, 2018-3₁ (2012).
11. Leshchiner, E. S. et al. Direct inhibition of oncogenic KRAS by hydrocarbon-stapled SOS1 helices. Proc Natl Acad Sci USA 112, 1761-6 (2015).
12. Takada, K. et al. Targeted disruption of the BCL9/beta-catenin complex inhibits oncogenic Wnt signaling. Sci TranslMed 4, 148ra117 (2012).
13. Bird, G. H., Crannell, W. C. & Walensky, L. D. Chemical synthesis of hydrocarbon-stapled peptides for protein interaction research and therapeutic targeting. Curr Protoc Chem Biol 3, 99-117 (2011).
14. Chu, Q. et al. Towards understanding cell penetration by stapled peptides. Med Chem Commun 6, 111-119 (2015).
15. Sun, T. L., Sun, Y., Lee, C. C. & Huang, H. W. Membrane permeability of hydrocarbon-cross-linked peptides. Biophys J 104, 1923-32 (2013).
16. Bird, G. H., Gavathiotis, E., LaBelle, J. L., Katz, S. G. & Walensky, L. D. Distinct BimBH3 (BimSAHB) stapled peptides for structural and cellular studies. ACS Chem Biol 9, 831-7 (2014).
17. Okamoto, T. et al. Further insights into the effects of pre-organizing the BimBH3 helix. ACS Chem Biol 9, 838-9 (2014).
18. Okamoto, T. et al. Stabilizing the pro-apoptotic BimBH3 helix (BimSAHB) does not necessarily enhance affinity or biological activity. ACS Chem Biol 8, 297-302 (2013).
19. Li, Y. C. et al. A versatile platform to analyze low-affinity and transient protein-protein interactions in living cells in real time. Cell Rep 9, 1946-58 (2014).
20. Edwards, A. L. et al. Cellular Uptake and Ultrastructural Localization Underlie the Pro-apoptotic Activity of a Hydrocarbon-stapled BIM BH3 Peptide. ACS Chem Biol 10, 2149-57 (2015).
21. Hotelling, H. Analysis of a complex of statistical variables into principal components. J Edu Psych 24, 417-441, 498-520 (1933).
22. Hotelling, H. Relations between two sets of variates. Biometrika 27, 321-377 (1936).
23. Pearson, K. On lines and planes of closest fit to systems of points in space. Philosphical Magazine 2, 559-572 (1901).
24. https://cran.r-project.org/web/packages/rpart/rpart.pdf.
25. Cohen, N. A. et al. A competitive stapled peptide screen identifies a selective small molecule that overcomes MCL-1-dependent leukemia cell survival. Chem Biol 19, 1175-86 (2012).
26. Koss, B. et al. Requirement for antiapoptotic MCL-1 in the survival of BCR-ABL B-lineage acute lymphoblastic leukemia. Blood 122, 1587-98 (2013).
27. Edwards, A. L. et al. Multimodal interaction with BCL-2 family proteins underlies the proapoptotic activity of PUMA BH3. Chem Biol 20, 888-902 (2013).
28. Stewart, M. L., Fire, E., Keating, A. E. & Walensky, L. D. The MCL-1 BH3 helix is an exclusive MCL-1 inhibitor and apoptosis sensitizer. Nat Chem Biol 6, 595-601 (2010).
29. Braun, C. R. et al. Photoreactive stapled BH3 peptides to dissect the BCL-2 family interactome. Chem Biol 17, 1325-33 (2010).
30. Bird, G. H. et al. Mucosal delivery of a double-stapled RSV peptide prevents nasopulmonary infection. J Clin Invest 124, 2113-24 (2014).
3₁. Bird, G. H. et al. Stapled HIV-1 peptides recapitulate antigenic structures and engage broadly neutralizing antibodies. Nat StructMol Biol 21, 1058-67 (2014).
32. LaRochelle, J. R., Cobb, G. B., Steinauer, A., Rhoades, E. & Schepartz, A. Fluorescence correlation spectroscopy reveals highly efficient cytosolic delivery of certain penta-arg proteins and stapled peptides. J Am Chem Soc 137, 2536-41 (2015).
33. Baek, S. et al. Structure of the stapled p53 peptide bound to Mdm2. JAm Chem Soc 134, 103-6 (2012).
34. Bird, G. H., Bemal, F., Pitter, K. & Walensky, L. D. Synthesis and biophysical characterization of stabilized alpha-helices of BCL-2 domains. Methods Enzymol 446, 369-86 (2008).
35. Ihaka, R. & Gentleman, R. A language for data analysis and graphics. J Comp Graph Stat (1996).
36. https://cran.r-project.org/web/packages/pvrank/pvrank.pdf.
37. StataCorp. 2013. Stata Statistical Software: Release 13. College Station, T. S. L.
38. Breiman, L., Friedman, J. H., Olshen, R. & Stone, C. J. Classification and regression trees. Wadsworth International Group, Belmont Calif. (1984).
39. Eisenberg, D. & McLachlan, A. D. Solvation energy in protein folding and binding. Nature 319, 199-203 (1986).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All patent applications, patents, and other publications cited herein are incorporated by reference in their entireties.

What is claimed is:

1. A method of making a cell-penetrant hydrocarbon-stapled and/or stitched peptide (HSP), the method comprising:
providing an alpha-helical peptide that binds a target protein;
generating a hydrocarbon-stapled and/or stitched peptide (HSP) of the alpha-helical peptide by placing a staple and/or a stitch at an amphipathic boundary of the alpha-helical peptide, thereby generating a HSP that is cell-penetrant.

2. The method of claim 1, wherein the alpha-helical peptide is 6 to 40 amino acids in length.

3. The method of claims 1 or 2, wherein the staple extends the hydrophobic surface beyond the target protein binding surface.

4. The method of any one of claims 1 to 3, wherein the HSP has a total internalized FITC intensity (TIFI) that is greater than 0.5×10⁶.

5. The method of any one of claims 1 to 4, wherein the HSP has a calculated hydrophobicity that is greater than 0.5.

6. The method of any one of claims 1 to 5, wherein the HSP has a high performance liquid chromatography (HPLC) retention time of 9.56 or greater at pH 7 or pH 4.

7. The method of any one of claims 1 to 6, wherein the HSP has a percent α-helicity of 61% to 86%.

8. The method of any one of claims 1 to 7, wherein the HSP has a net charge of +2 to −1.

9. The method of any one of claims 1-8, wherein the HSP is derived from an alpha-helical peptide from an anti-apoptotic or a pro-apoptotic BCL-2 family protein.

10. A cell-penetrant hydrocarbon-stapled and/or stitched peptide (HSP) comprising:
a hydrocarbon-stapled and/or stitch peptide (HSP); wherein a staple and/or stitch is located at an amphipathic boundary of the HSP, and wherein the HSP is cell-penetrant.

11. The HSP of claim 10, wherein the HSP has a total internalized FITC intensity (TIFI) that is greater than 0.5×10⁶.

12. The HSP of claims 10 or 11, wherein the HSP has a calculated hydrophobicity that is greater than 0.5.

13. The HSP of any one of claims 10-12, wherein the HSP has a high performance liquid chromatography (HPLC) retention time of 9.56 minutes or greater at pH 7 or pH 4.

14. The HSP of any one of claims 10-13, wherein the HSP has a percent α-helicity of 61% to 86%.

15. The HSP of any one of claims 10-14, wherein the HSP has a net charge of +2 to −1.

16. The HSP of any one of claims 10-15, wherein the HSP is 6 to 40 amino acids in length.

17. The HSP of any one of claims 10-16, wherein the HSP binds an anti-apoptotic or a pro-apoptotic protein of the BCL-2 family.

18. A method of selecting a hydrocarbon-stapled and/or stitched peptide (HSP), the method comprising:
providing a library of HSPs;
assessing the hydrophobicity of the HSPs in the library; and
selecting an HSP having an overall cellular uptake-facilitating level of hydrophobicity.

19. The method of claim 18, wherein the hydrophobic surface area of the selected HSP extends beyond the interaction site of the selected HSP with its target.

20. The method of claims 18 or 19, wherein the overall cellular uptake-facilitating level of hydrophobicity corresponds to an HPLC retention time of about 9.7 to about 11.2 minutes.

21. The method of any of claims 18-20, wherein the selected HSP comprises a staple and/or a stitch at the amphipathic boundary of the HSP.

22. The method of any of claims 18-21, further comprising assessing the charge of the HSPs in the library and selecting an HSP having an overall cellular uptake-facilitating charge.

23. The method of claim 22, wherein the isoelectric point is about 8.8 to about 9.34.

24. The method of any of claims 18-23, further comprising assessing the cell permeability of the HSPs in the library and selecting an HSP with high cell permeability.

25. The method of any of claims 18-24, further comprising assessing the cell lytic activity of the HSPs in the library and selecting an HSP with low or no cell lytic activity.

26. The method of any of claims 18-25, further comprising assessing the α-helicity of the HSPs in the library and selecting an HSP having an overall cellular uptake-facilitating level of α-helicity.

27. The method of claim 26, wherein the α-helicity is 61% to 86%.

28. The method of claim 26, wherein the α-helicity is 40% to 90%.

29. The method of any of claims 18-28, wherein the library is a staple walk library.

30. The method of any of claims 18-29, wherein the library is a point mutant library.

31. A method of selecting a hydrocarbon-stapled and/or stitched peptide (HSP), the method comprising:
providing a library of HSPs;
assessing the hydrophobicity of the HSPs in the library;
selecting one or more HSPs having an overall cellular uptake-facilitating level of hydrophobicity;
assessing at least one of the α-helicity, cell permeability, charge, isoelectric point, and/or cell membrane lytic activity of the one or more selected HSPs; and
further selecting one or more HSPs with an overall cellular uptake-facilitating α-helicity, an overall cellular uptake-facilitating charge, an overall cellular uptake-facilitating isoelectric point, high cell permeability, and/or low cell lytic activity.

32. A method of identifying tolerated sites for diversification within a hydrocarbon-stapled and/or stitched peptide (HSP), the method comprising:
providing an initial HSP;
substituting one or more amino acids in the initial HSP with alanine, glutamate, aspartate, arginine, and/or lysine to generate a library of HSP variants;
assessing at least one of hydrophobicity, α-helicity, cell permeability, charge, isoelectric point, and/or cell lytic activity of the library of HSP variants; and
selecting one or more HSP variants with hydrophobicity, α-helicity, cell permeability, charge, isoelectric point, and/or cell lytic activity similar to that of the initial HSP.

33. A method of making a hydrocarbon-stapled and/or stitched peptide (HSP), the method comprising:
synthesizing an HSP having an overall cellular uptake-facilitating level of hydrophobicity.

34. The method of claim 33, wherein the hydrophobic surface area of the HSP extends beyond the interaction site of the HSP with its target.

35. The method of claim 33 or 34, wherein the level of hydrophobicity corresponds to an HPLC retention time of about 9.7 to about 11.2 minutes.

36. The method of any of claims 33-35, wherein the HSP has an overall cellular uptake-facilitating level of α-helicity.

37. The method of claim 36, wherein the overall α-helicity the HSP is 61% to 86%.

38. The method of any of claims 33-37, wherein the HSP has a cellular uptake-facilitating isoelectric point.

39. The method of claim 38, wherein the isoelectric point is about 8.8 to about 9.34.

40. The method of any of claims 33-39, wherein the HSP comprises a staple and/or a stitch at the amphipathic boundary of the HSP.

41. The method of any of claims 33-40, wherein the HSP has high cell permeability.

42. The method of any of claims 34-41, wherein the HSP has low or no cell lytic activity.

43. A method of making a hydrocarbon-stapled and/or stitched peptide (HSP), the method comprising:
synthesizing an HSP having an overall cellular uptake-facilitating level of hydrophobicity, an overall cellular uptake-facilitating level of α-helicity, and a cellular uptake-facilitating isoelectric point;
wherein the HSP comprises a staple and/or a stitch at the amphipathic boundary of the HSP;
wherein the HSP has high cell permeability; and
wherein the HSP has low or no cell lytic activity.

44. A method of determining the cell-penetrance of a hydrocarbon-stapled and/or stitched peptide (HSP), the method comprising:
(a) providing a hydrocarbon-stapled and/or stitched peptide (HSP);
(b) calculating at least one biophysical property of the HSP; wherein the at least one biophysical property is hydrophobicity, high performance liquid chromatography (HPLC) retention time, percent α-helicity, or net charge; and
(c) determining the cell-penetrance of the HSP based on the at least one biophysical property.

45. The method of claim 44, wherein the at least one biophysical property of the HSP is hydrophobicity, and the method comprises:
determining hydrophobicity of the HSP; and
determining that either:
(i) the HSP has a calculated hydrophobicity that is greater than 0.5, and the HSP is cell-penetrant; or
(ii) the HSP had a calculated hydrophobicity that is less than 0.5; and the HSP is not cell-penetrant.

46. The method of claim 44 or 45, wherein the at least one biophysical property of the HSP is HPLC retention time, and determining that either:
(i) the HPLC retention time of the HSP is less than 9.56 minutes at pH 7 or pH 4, and the HSP is not cell-penetrant; or
(ii) the HPLC retention time of the HSP is equal to or greater than 9.56 minutes at pH 7 or pH 4, and the HSP is cell-penetrant.

47. The method of any one of claims 44-46, wherein the at least one biophysical property of the HSP is percent α-helicity, and determining that either:
(i) the percent α-helicity of the HSP is less than 20% or greater than 90%, and the HSP is not cell-penetrant; or
(ii) the percent α-helicity of the HSP is 61% to 86%, and the HSP is cell-penetrant.

48. The method of any one of claims 44-47, wherein the method further comprises determining that the net charge of the HSP is +2 to −1.

49. The method of any one of claims 44-48, wherein the method further comprises determining the total internalized FITC intensity (TIFI) of the HSP.

50. The method of claim 49, wherein a TIFI that is greater than 0.5×10⁶ indicates that the HSP is cell-penetrant.

51. The method of claim 49, wherein a TIFI that is less than 0.5×10⁶ indicates that the HSP is not cell-penetrant.

52. A method of optimizing cell-penetrance of a hydrocarbon-stapled and/or stitched peptide, the method comprising:
(a) providing a first hydrocarbon-stapled and/or stitched peptide (HSP) that binds a target protein;
(b) generating a second HSP that is identical to the first HSP except at one or more amino acid positions, wherein the second HSP binds to the same target as the first HSP, and has at least one altered biophysical property compared to the first HSP; wherein the at least one biophysical property is selected from the group consisting of: hydrophobicity, high performance liquid chromatography (HPLC) retention time, percent α-helicity, or net charge; and wherein either
(i) the HPLC retention time of the second HSP is altered compared to the first HSP, and the second HSP has improved cellular uptake as compared to the first HSP;
(ii) the hydrophobicity of the second HSP is altered compared to the first HSP, and the second HSP has improved cellular uptake as compared to the first HSP;
(iii) the net charge of the second HSP is altered compared to the first HSP, and the second HSP has improved cellular uptake as compared to the first HSP; or
(iv) the percent α-helicity of the second HSP is altered compared to the first HSP, and the second HSP has improved cellular uptake as compared to the first HSP.

53. The method of claim 52, wherein the second HSP has increased retention time as compared to the first HSP, and the second HSP has improved cellular uptake as compared to the first HSP.

54. The method of any one of claims 52 to 53, wherein the second HSP binds to the same target as the first HSP with the same or greater binding affinity to the target as compared to the first HSP.

55. The method of any one of claims 52 to 54, wherein the second HSP has the same or reduced effect on non-specific cell lysis.

56. The method of any one of claims 52 to 55, wherein the HSP is 6 to 40 amino acids in length.

57. A method of selecting a cell-penetrant hydrocarbon-stapled and/or stitched peptide (HSP) that does not exhibit non-specific cell lytic activity, the method comprising:
(a) providing a hydrocarbon-stapled and/or stitched alpha-helical peptide (HSP) that binds a target protein;
(b) determining percent α-helicity or isoelectric point (pI) of the HSP; and
(c) selecting the HSP as not exhibiting non-specific cell lytic activity, when the HSP has:
(i) an isoelectric point (pI) that is less than 9.76; and
(ii) a percent α-helicity that ranges from 21% to 96%.

58. The method of claim 57 further comprising determining that the hydrophobicity of the HSP is greater than 0.5.

59. The method of claim 57 further comprising determining that the HPLC retention time of the HSP is equal to or greater than 9.56 minutes at pH 7 or pH 4.

60. The method of claim 57 further comprising determining that the percent α-helicity of the HSP is 61% to 86%.

61. The method of claim 57 further comprising determining that the pI of the HSP is less than 9.75.

62. A method of determining the cell-penetrance and non-specific cell lysis activity of a hydrocarbon-stapled and/or stitched peptide (HSP), the method comprising:
(a) providing a hydrocarbon-stapled and/or stitched alpha-helical peptide (HSP) that binds a target protein;
(b) determining at least one biophysical property of the HSP; wherein the at least one biophysical property is hydrophobicity, high performance liquid chromatography (HPLC) retention time, percent α-helicity, net charge or isoelectric point (pI); and
(c) determining the cell-penetrance and the non-specific cell lysis activity of the HSP based on the at least one biophysical property.

63. The method of claim 62, wherein the at least one biophysical property of the HSP is isoelectric point (pI).

64. The method of claim 62, wherein the at least one biophysical property of the HSP is HPLC retention time.

65. The method of claim 62, wherein the at least one biophysical property of the HSP is calculated hydrophobicity.

66. The method of claim 62, wherein the at least one biophysical property of the HSP is percent α-helicity.

67. The method of claim 62, wherein the at least one biophysical property of the HSP is pI and HPLC retention time.

68. The method of claim 62, wherein the at least one biophysical property of the HSP is pI and calculated hydrophobicity.

69. The method of claim 62, wherein the at least one biophysical property of the HSP is pI and percent α-helicity.

70. The method of claim 62, wherein the at least one biophysical property of the HSP is HPLC retention time and percent α-helicity.

71. The method of claim 62, wherein the at least one biophysical property of the HSP is HPLC retention time and net charge.

72. The method of claim 62, wherein the at least one biophysical property of the HSP is HPLC retention time, pI, and net charge.

73. The method of claim 62, wherein the at least one biophysical property of the HSP are isoelectric point (pI), HPLC retention time and percent α-helicity.

74. The method of claim 62, wherein the at least one biophysical property of the HSP is isoelectric point (pI), percent α-helicity, and net charge.

75. The method of claim 62, wherein the at least one biophysical property of the HSP is isoelectric point (pI), percent α-helicity, and calculated hydrophobicity.

76. The method of any one of claims 62 to 75, wherein the pI of the HSP is less than 9.76.

77. The method of any one of claims 62 to 75, wherein the HSP has a percent α-helicity between 21% to 96%.

78. The method of any one of claims 62 to 75, wherein the HSP has a HPLC retention time at pH 4 or 7 of 9.56 minutes or greater.

79. The method of any one of claims 62 to 75, wherein the HSP has a net charge of +2 to −1.

80. A pharmaceutical composition comprising the HSP of any one of claims 10-19.


SEQUENCE LISTING (51 sequences)

SEQ ID NO: 1 - protein, 21 aa, Artificial Sequence
  Description of Artificial Sequence Synthetic peptide
IWIAQELRRIGDEFNAYYARR

SEQ ID NO: 2 - protein, 21 aa, Artificial Sequence
  Description of Artificial Sequence Synthetic peptide
  MOD_RES (1)..(1): S-pentenyl alanine
  MOD_RES (5)..(5): S-pentenyl alanine
AWIAAELRRIGDEFNAYYARR

SEQ ID NO: 3 - protein, 21 aa, Artificial Sequence
  Description of Artificial Sequence Synthetic peptide
  MOD_RES (2)..(2): S-pentenyl alanine
  MOD_RES (6)..(6): S-pentenyl alanine
IAIAQALRRIGDEFNAYYARR

SEQ ID NO: 4 - protein, 21 aa, Artificial Sequence
  Description of Artificial Sequence Synthetic peptide
  MOD_RES (3)..(3): S-pentenyl alanine
  MOD_RES (7)..(7): S-pentenyl alanine
IWAAQEARRIGDEFNAYYARR

SEQ ID NO: 5 - protein, 21 aa, Artificial Sequence
  Description of Artificial Sequence Synthetic peptide
  MOD_RES (4)..(4): S-pentenyl alanine
  MOD_RES (8)..(8): S-pentenyl alanine
IWIAQELARIGDEFNAYYARR

SEQ ID NO: 6 - protein, 21 aa, Artificial Sequence
  Description of Artificial Sequence Synthetic peptide
  MOD_RES (5)..(5): S-pentenyl alanine
  MOD_RES (9)..(9): S-pentenyl alanine
IWIAAELRAIGDEFNAYYARR

SEQ ID NO: 7 - protein, 21 aa, Artificial Sequence
  Description of Artificial Sequence Synthetic peptide
  MOD_RES (6)..(6): S-pentenyl alanine
  MOD_RES (10)..(10): S-pentenyl alanine
IWIAQALRRAGDEFNAYYARR

SEQ ID NO: 8 - protein, 21 aa, Artificial Sequence
  Description of Artificial Sequence Synthetic peptide
  MOD_RES (7)..(7): S-pentenyl alanine
  MOD_RES (11)..(11): S-pentenyl alanine
IWIAQEARRIADEFNAYYARR

SEQ ID NO: 9 - protein, 21 aa, Artificial Sequence
  Description of Artificial Sequence Synthetic peptide
  MOD_RES (8)..(8): S-pentenyl alanine
  MOD_RES (12)..(12): S-pentenyl alanine
IWIAQELARIGAEFNAYYARR

SEQ ID NO: 10 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIGDAFNAYYARR

SEQ ID NO: 11 - protein, 21 aa, Artificial Sequence
  Description of Artificial Sequence Synthetic peptide
  MOD_RES (10)..(10): S-pentenyl alanine
  MOD_RES (14)..(14): S-pentenyl alanine
IWIAQELRRAGDEANAYYARR

SEQ ID NO: 12 - protein, 21 aa, Artificial Sequence
  Description of Artificial Sequence Synthetic peptide
  MOD_RES (11)..(11): S-pentenyl alanine
  MOD_RES (15)..(15): S-pentenyl alanine
IWIAQELRRIADEFAAYYARR

SEQ ID NO: 13 - protein, 21 aa, Artificial Sequence
  Description of Artificial Sequence Synthetic peptide
  MOD_RES (12)..(12): S-pentenyl alanine
  MOD_RES (16)..(16): S-pentenyl alanine
IWIAQELRRIGAEFNAYYARR

SEQ ID NO: 14 - protein, 21 aa, Artificial Sequence
  Description of Artificial Sequence Synthetic peptide
  MOD_RES (13)..(13): S-pentenyl alanine
  MOD_RES (17)..(17): S-pentenyl alanine
IWIAQELRRIGDAFNAAYARR

SEQ ID NO: 15 - protein, 21 aa, Artificial Sequence
  Description of Artificial Sequence Synthetic peptide
  MOD_RES (14)..(14): S-pentenyl alanine
  MOD_RES (18)..(18): S-pentenyl alanine
IWIAQELRRIGDEANAYAARR

SEQ ID NO: 16 - protein, 21 aa, Artificial Sequence
  Description of Artificial Sequence Synthetic peptide
  MOD_RES (15)..(15): S-pentenyl alanine
  MOD_RES (19)..(19): S-pentenyl alanine
IWIAQELRRIGDEFAAYYARR

SEQ ID NO: 17 - protein, 21 aa, Artificial Sequence
  Description of Artificial Sequence Synthetic peptide
  MOD_RES (16)..(16): S-pentenyl alanine
  MOD_RES (20)..(20): S-pentenyl alanine
IWIAQELRRIGDEFNAYYAAR

SEQ ID NO: 18 - protein, 21 aa, Artificial Sequence
  Description of Artificial Sequence Synthetic peptide
  MOD_RES (17)..(17): S-pentenyl alanine
  MOD_RES (21)..(21): S-pentenyl alanine
IWIAQELRRIGDEFNAAYARA

SEQ ID NO: 19 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIALELRAIGDAFNAYYARR

SEQ ID NO: 20 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQLLRAIGDAFNAYYARR

SEQ ID NO: 21 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELLAIGDAFNAYYARR

SEQ ID NO: 22 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIGLAFNAYYARR

SEQ ID NO: 23 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
ILIAQELRAIGDAFNAYYARR

SEQ ID NO: 24 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIGDAFNLYYARR

SEQ ID NO: 25 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIGDAFNALYARR

SEQ ID NO: 26 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IEIAQELRAIGDAFNAYYARR

SEQ ID NO: 27 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIGDAFNAEYARR

SEQ ID NO: 28 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIGDAFNRYYARR

SEQ ID NO: 29 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIGDAFNARYARR

SEQ ID NO: 30 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELDAIGDAFNAYYARR

SEQ ID NO: 31 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAEGDAFNAYYARR

SEQ ID NO: 32 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIGDAFNAYYTRR

SEQ ID NO: 33 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IRIAQELRAIGDAFNAYYARR

SEQ ID NO: 34 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIGDAFNEYYARR

SEQ ID NO: 35 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIGDAFNATYARR

SEQ ID NO: 36 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIEDAFNAYYARR

SEQ ID NO: 37 - protein, 21 aa, Artificial Sequence
  Description of Artificial Sequence Synthetic peptide
  MOD_RES (9)..(9): S-pentenyl alanine
  MOD_RES (13)..(13): S-pentenyl alanine
IWIAQELRAIGDAVNAYYARR

SEQ ID NO: 38 - protein, 14 aa, Artificial Sequence
  Description of Artificial Sequence Synthetic peptide
  MOD_RES (4)..(4): R-octenyl alanine
  MOD_RES (10)..(10): Cyclobutylalanine
  MOD_RES (11)..(11): S-pentenyl alanine
LTFAEYWANAASAA

SEQ ID NO: 39 - protein, 16 aa, Artificial Sequence
  Description of Artificial Sequence Synthetic peptide
FFGIYLTNILKTEEGN

SEQ ID NO: 40 - protein, 16 aa, Artificial Sequence
  Description of Artificial Sequence Synthetic peptide
  MOD_RES (1)..(1): R-octenyl alanine
  MOD_RES (8)..(8): S-pentenyl alanine
AFGIYLTAILKTEEGN

SEQ ID NO: 41 - protein, 16 aa, Artificial Sequence
  Description of Artificial Sequence Synthetic peptide
  MOD_RES (2)..(2): R-octenyl alanine
  MOD_RES (9)..(9): S-pentenyl alanine
FAGIYLTNALKTEEGN

SEQ ID NO: 42 - protein, 16 aa, Artificial Sequence
  Description of Artificial Sequence Synthetic peptide
  MOD_RES (3)..(3): R-octenyl alanine
  MOD_RES (10)..(10): S-pentenyl alanine
FFAIYLTNIAKTEEGN

SEQ ID NO: 43 - protein, 16 aa, Artificial Sequence
  Description of Artificial Sequence Synthetic peptide
  MOD_RES (4)..(4): R-octenyl alanine
  MOD_RES (11)..(11): S-pentenyl alanine
FFGAYLTNILATEEGN

SEQ ID NO: 44 - protein, 16 aa, Artificial Sequence
  Description of Artificial Sequence Synthetic peptide
  MOD_RES (5)..(5): R-octenyl alanine
  MOD_RES (12)..(12): S-pentenyl alanine
FFGIALTNILKAEEGN

SEQ ID NO: 45 - protein, 16 aa, Artificial Sequence
  Description of Artificial Sequence Synthetic peptide
  MOD_RES (6)..(6): R-octenyl alanine
  MOD_RES (13)..(13): S-pentenyl alanine
FFGIYATNILKTAEGN

SEQ ID NO: 46 - protein, 16 aa, Artificial Sequence
  Description of Artificial Sequence Synthetic peptide
  MOD_RES (7)..(7): R-octenyl alanine
  MOD_RES (14)..(14): S-pentenyl alanine
FFGIYLANILKTEAGN

SEQ ID NO: 47 - protein, 16 aa, Artificial Sequence
  Description of Artificial Sequence Synthetic peptide
  MOD_RES (8)..(8): R-octenyl alanine
  MOD_RES (15)..(15): S-pentenyl alanine
FFGIYLTAILKTEEAN

SEQ ID NO: 48 - protein, 16 aa, Artificial Sequence
  Description of Artificial Sequence Synthetic peptide
  MOD_RES (9)..(9): R-octenyl alanine
  MOD_RES (16)..(16): S-pentenyl alanine
FFGIYLTNALKTEEGA

SEQ ID NO: 49 - protein, 17 aa, Artificial Sequence
  Description of Artificial Sequence Synthetic peptide
  MOD_RES (10)..(10): R-octenyl alanine
  MOD_RES (17)..(17): S-pentenyl alanine
FFGIYLTNIAKTEEGNA

SEQ ID NO: 50 - protein, 21 aa, Artificial Sequence
  Description of Artificial Sequence Synthetic peptide
  MOD_RES (2)..(2): Glu, Leu or Arg
  MOD_RES (8)..(8): Leu or Asp
  MOD_RES (16)..(16): Leu, Glu or Arg
  MOD_RES (17)..(17): Thr, Glu, Leu or Arg
IXIALELXREELEVNXXYTRR

SEQ ID NO: 51 - protein, 21 aa, Artificial Sequence
  Description of Artificial Sequence Synthetic peptide
  MOD_RES (2)..(2): Leu, Glu or Arg
  MOD_RES (8)..(8): Leu or Asp
  MOD_RES (16)..(16): Leu, Glu or Arg
  MOD_RES (17)..(17): Thr, Glu, Arg or Leu
IXIALLLXREELEVNXXYTRR